SEQUENCE LISTING (7 sequences)

SEQ ID NO: 1 - DNA, 80 bp, Artificial Sequence
  Synthetic Primer
ctaagttgccttttcctttcaggagttttgctctgcatcaggggaaccttgacgcccaggatggtgagcaagggcgagga

SEQ ID NO: 2 - DNA, 81 bp, Artificial Sequence
  Synthetic Primer
tatggcaggcatgcgactagaaagatccgcttgtcacctgtagggtgtggggttcacgacgggaacaaaagctggagctcg

SEQ ID NO: 3 - DNA, 85 bp, Artificial Sequence
  Synthetic Primer
ctaagttgccttttcctttcaggagttttgctctgcatcaggggaaccttgacgcccaggatgtccaatttactgaccgtacacc

SEQ ID NO: 4 - DNA, 81 bp, Artificial Sequence; identical to SEQ ID NO: 2
  Synthetic Primer

SEQ ID NO: 5 - DNA, 12 bp, Artificial Sequence
  Synthetic Primer
caggtgtgagcc

SEQ ID NO: 6 - DNA, 12 bp, Artificial Sequence
  Synthetic Primer
tggggtgtggag

SEQ ID NO: 7 - DNA, 12 bp, Artificial Sequence
  Synthetic Primer
ggaggtgtgaat

(19) United States
(12) Patent Application Publication
(10) Pub. No.: US 2015/0164957 A1
(43) Pub. Date: Jun. 18, 2015

(54) CONVERSION OF CARDIOMYOCYTES INTO FAST CONDUCTING CARDIOMYOCYTES OR SLOW CONDUCTING NODAL CELLS

(71) Applicant: The University of Chicago, Chicago, IL (US)

(72) Inventors: David Arnolds, Chicago, IL (US); Ivan Moskowitz, Chicago, IL (US)

(21) Appl. No.: 14/401,915

(22) PCT Filed: May 17, 2013
(86) PCT No.: PCT/US13/41593

§ 371 Date: Nov. 18, 2014

Related U.S. Application Data
(60) Provisional application No. 61/648,668, filed on May 18, 2012.

(51) Int. Cl. A61K 35/34; G01N 33/50; C12N 5/077

(52) U.S. Cl. CPC A61K 35/34; C12N 5/0657; G01N 33/5026

(57) ABSTRACT

Tbx5 and Tbx3 were shown to be key determinants of regional identity in the AVCS. Various embodiments relate to increasing the amount of Tbx5 or Tbx3 in a cardiomyoctye whereby the cardiomycyte is converted to a fast conducting cardiomyocyte or a slow conducting nodal cell, respectively. Further embodiments employ the fast conducting cardiomyocytes and nodal cells in therapeutic methods and in methods for screening compounds for effect on these cell types.

This application claims the benefit of priority to U.S. Provisional Patent Application Ser. No. 61/648,668, filed May 18, 2012, hereby incorporated by reference in its entirety.

### BACKGROUND

1. Field of the Invention

The present invention relates generally to the fields of molecular biology, cellular biology, and cardiology. More particularly, it relates to the preparation and use of fast conducting cardiomyocytes and slow conducting nodal cells.

2. Description of Related Art

The cardiac conduction system (CCS) is essential to ensure a regular heartbeat, coordinated contraction of the heart, and ultimately efficient circulation of blood throughout the organism. Despite the clinical consequences of conduction system disease, relatively little is known about the transcriptional networks that are responsible for specifying the conduction system early in development and for maintaining the specialized functions of the mature cardiac conduction system.

### SUMMARY OF THE INVENTION

Various embodiments provide a method of making a fast conducting cardiomyocyte comprising: (a) obtaining a cardiomyocte; and (b) increasing Tbx5 in the cardiomyocyte thereby coverting the cardiomyocyte into a fast conducting cardiomyocyte.

Other embodiments provide a method of making a slow conducting nodal cell comprising: (a) obtaining a cardiomyocte; and (b) increasing Tbx3 in the cardiomyocyte thereby coverting the cardiomyocyte into a slow conducting nodal cell.

In certain aspects, the cardiomyoctye is a primary cardiomyocyte, a cardiomyocyte cell line, or a cardiomyocyte that has been differentiated from a stem cell such as an embryonic stem cell or an induced pluripotent stem cell.

In some embodiments, the methods of making fast conducting cardiomyocyte or nodal cells further comprises differentiating a stem cell, such as an embryonic stem (ES) cell or an induced pluripotent stem (iPS) cell, into a cardiomyocyte. In certain aspects the cells are mammal cells. The cells may be, for example, primate or rodent cells. In certain embodiments, the cells are human, pig, dog, mouse, or rat.

Tbx5 or Tbx3 can be increased in a cell by a variety of techniques known to those in the art. In some aspects, the increase is achieved by overexpressing Tbx5 or Tbx3 in the cell. Overexpression may be achieved by, for example, upregulating the expression of an endogenous Tbx5 or Tbx3 gene, or by expressing an exogenous nucleic acid sequence encoding Tbx5 or Tbx3 in the cell. In certain aspects, the exogenous nucleic acid sequence is a cDNA or an mRNA.

Methods for delivering nucleic acids to cells include, but are not limited to, the use of vectors, such as viruses and plasmids, lipofection, electropolation, electroporation, microinjection, and the like. Examples of viral vectors, which are replication-deficient, include, but are not limited to, retrovirus vectors, lentivirus vectors, adenovirus vectors, adeno-associated virus vectors. In some embodiments, the exogenous nucleic acid sequence e is integrated into the genome of the cell. The nucleic acid sequences may be operably linked to a regulatory sequence(s), such as promoter and enhancer, so that they are capable of being expressed. The vectors may further comprise a positive selectable marker(s), such as a drug resistance gene(s) (e.g., puromycin resistance gene, neomycin resistance gene, ampicillin resistance gene, hygromycin resistance gene, or the like), a negative selectable marker(s) (e.g., diphtheria toxin A fragment gene, thymidine kinase gene, or the like), an internal ribosome entry site (IRES), a terminator, an origin of replication, etc. The cells may then be assessed for fast conducting cardiomyocyte or a slow conducting nodal cell phenotypes.

In other embodiments, the increase in Tbx5 or Tbx3 is achieved by introducing the Tbx5 or Tbx3 protein, or a functional fragment thereof, into the cell. Proteins may enter cells via the endocytic pathway. Additionally, a variety of reagents for the delivery of proteins into mammalian cells have been developed including lipid-linked compounds, nanoparticles, fusion to receptor ligands, and fusion to protein transduction domains including the HIV-1 transactivator of transcription (Tat) peptide, oligoarginine, and the Drosophila Antennapedia-derived penetratin peptide.

In certain aspects, the fast conducting cardiomyocytes are selected from the group consisting of atrioventricular bundle cells, bundle branch cells, and Perkinje cells. Various embodiments provide a composition comprising one or more of the atrioventricular bundle cells, bundle branch cells, and/or Perkinje cells produced from a cardiomyocyte in which Txb5 was increased to convert the cardiomycyte into a fast conducting cardiomyocyte. In some embodiments, the fast conducting cardiomyocytes are separated from cardiomyocytes that were not converted to fast conducting cardiomyocytes.

In certain aspects, the nodal cells are selected from the group consisting of AV nodal cells and SA nodal cells. Various embodiments provide a composition comprising one or more AV nodal cells and/or SA nodal cells produced from a cardiomyocyte in which Txb3 was increased to convert the cardiomycyte into a nodal cell. In some embodiments, the nodal cells are separated from cardiomyocytes that were not converted to nodal cells.

Other embodiments provide methods of screening a compound for an effect on a fast conducting cardiomyocyte cell culture, comprising: (a) contacting the cell culture with the compound; and (b) observing a change in a fast conducting cardiomyocyte in the cell culture compared to a fast conducting cardiomyocyte in an untreated cell culture and (c) determining a difference between the treated and untreated fast conducting cardiomyocytes in the respective cell cultures.

Further embodiments provide methods of screening a compound for an effect on a slow conducting nodal cell culture, comprising: (a) contacting the cell culture with the compound; and (b) observing a change in a nodal cell in the cell culture compared to a nodal cell in an untreated cell culture and (c) determining a difference between the treated and untreated nodal cells in the respective cell cultures.

The compound used for screening may be any compound of interest with respect to its effect on fast conducting cardiomyocytes and/or nodal cells. The compound may be a drug, such as a calcium channel blocker, a β-adrenoreceptor agonist, or an α-adrenoreceptor agonist. The compound may be, for example, a small molecule, a peptide, an oligonucleotide, or a toxin.

Other embodiments provide methods for treating a subject with heart disease, comprising transplanting the fast conducting cardiomyocyte as disclosed herein to the heart of the subject.

Further embodiments provide a method for treating a subject with heart disease, comprising transplanting the nodal cells as disclosed herein to the heart of the subject.

The subject may be a mammal. In some embodiments, the subject is a human. In certain embodiments, the cells used in the treatement were differentiated from iPS cells. In some embodiments, the iPS cells were derived from the subject's somatic cells.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Schematic representation of the structure of the cardiac conduction system and its correlation to the surface electrocardiogram (ECG). (A) Impulse propagation through the mammalian heart can be traced through the surface ECG. (B) The cardiac conduction system is comprised of the sino-atrial node (SAN), AV node (AVN), AV bundle, bundle branches, and Purkinje fibers.

FIGS. 2A-2D. Creation of minKGFP BAC and validation of GFP expression in the developing and mature conduction system. RedET recombination in E. coli was used to replace the minK coding exon with an EGFP-pa-FRT-Zeo^{R}-FRT construct; the Zeo^{r} cassette was subsequently removed via FLP activity (A). Hearts from minKGFP BAC transgenic mice were examined via epifluouresence (EGFP) and under brightfield illumination (merged with EGFP; Merge) at E10.5 (B), E14.5 (C), and in adult mice (D) and compared to β-galactosidase activity in minK^{lacZ} knockin mice. EGFP expression was consistently observed in the AV canal/AV node (arrowheads), primary ring/AV bundle (double headed arrow), left and right bundle branchese (half-filled arrowheads), and in the outflow tract (arrow) in a similar distribution to B-galactosidase activity in minK^{lacZ} knockin mice.

FIGS. 3A-3C. FACS sorting and transcriptional profiling strategy to isolate cells with high levels of minKGFP expression. E10.5 or E14.5 hearts were digested to single cells and sorted on the basis of GFP fluorescence intensity into GFP negative, GFP low, and GFP high groups, as described in the text (A). RT-PCR using the indicated markers demonstrated that GFP negative, low, and high fractions are enriched for non-cardiomyocytes, chamber myocardium, and conduction system cells, respectively (B). Transcriptional profiling of E10.5 hearts was performed using Agilent microarrays (C).

FIGS. 4A-4F. In situ hybridization validates E10.5 CCS expression of BMP pathway genes identified in the microarray. Slide in situ hybridization demonstrated expression of Bmp-2 (A) and Id2 (B) in the AV ring and primary ring at E10.5. Whole mount in situ hybridization demonstrated primary ring expression (arrows) of Slc6a4 (C), Gsbs (D), Gpcl (E), and Adm (F).

FIGS. 5A-5L. Removal of SMAD4 in the mef2c-AHF-Cre domain affects molecular specification of the AV bundle. Slide in situ hybridization was performed on E14.5 Smad4^{fl/} (A, B, E, F, I, J) and Smad4^{mef2c-AHF-Cre/-} littermates (C, D, G, H, K, L) for Id2 (A-d), Tbx3 (E-H) and Cx43 (I-L). Loss of Smad4 resulted in reductions in Id2 and Tbx3 expression in the embryonic AV bundle; Cx43 expression was unaffected by the absence of Smad4. Boxed regions in A, E, G, I, and K are presented at higher magnification in B, D, F, H, J, and L.

FIGS. 6A-6C. Removal of Smad4 in the mef2c-AHF-Cre domain does not affect A-V interval or ventricular activation pattern. Optical mapping was performed on Smad4^{fl/-} (n=22) and Smad44^{mef2c-AHF-Cre/-} (n=12) hearts at E12.5. The A-V interval (A) and ventricular activation patterns (representative activation maps shown in B-C) were not different between genotypes. Data shown in A represents mean+/−standard deviation. Scale bar in B and C represents 500 uM.

FIG. 7. Recombineering strategy used to create minKCreER^{T2} BAC. RedET recombination in E. coli was used to replace the minK coding exon with a CreER^{T2}-pA-FRT-Zeo^{R}-FRT construct. The Zeo^{r} cassette was subsequently removed via FLP activity. The resulting BAC was then purified, digested with NotI, and used for pronuclear injection of fertilized oocytes. Blue lines indicate NotI sites, minK exons are indicated with solid black rectangles, and FRT sites are indicated with grey ovals.

FIGS. 8A-8D. Tamoxifen-inducible cre activity in the AV bundle and bundle branches of minK:CreER^{T2};ROSA26R double transgenic mice. Hearts from mice receiving (A-B) or not receiving (C-D) tamoxifen (TMX) were dissected to reveal the left (LV; A,C) and right (RV; B,D) ventricular septal surfaces and subsequently were stained with X-Gal to detect β-galactosidase activity (blue). β-galactosidase activity was detected in the AV bundle (A, B) and bundle branches (A, B) of mice that received tamoxifen, while only scattered blue cells (arrowheads) were found in the absence of tamoxifen (C, D). AVB: AV bundle; LBB: left bundle branch; RBB: right bundle branch.

FIGS. 9A-9O. Tamoxifen-inducible cre activity in the molecularly defined AV node, bundle, and bundle branches of minK:CreER^{T2};ROSA26R double transgenic mice. Near-adjacent frozen sections from the AV node (AVN; A-E), AV bundle (AVB; F-J) and bundle branches (BB; K-O) of minK:CreER^{T2};ROSA26R double transgenic mice receiving tamoxifen were stained for X-Gal (blue; A, B, F, G, K, L), Acetylcholinesterase (Ache) activity (brown; C, H, M), HCN4 (green; D, I, N), or Cx40 (green; E, J, O) and Cx43 (red; E, J, O). Sections stained for X-Gal were counterstained with Nuclear Fast Red and sections stained for AchE activity were counterstained with hematoxylin. Sections were photographed at 4× (A, F, K) or 40× magnification (B-E, G-J, L-O). Rectangles in A, F, and K indicate the areas presented at higher magnification in B-E, G-J, and L-O. Arrow in A indicates X-Gal positive cells surrounding a coronary vessel (cv) shown at higher magnification in the inset. rv: right ventricle; vs: ventricular septum; lv: left ventricle.

FIGS. 10A-10C. Tamoxifen-inducible recombination in E13.5 minK:CreER^{T2};ROSA26R double transgenic embryos. Tamoxifen was administered at E9.5 to timed pregnant females, and pups were evaluated via X-Gal staining at E13.5 by whole mount (A: anterior view; B: posterior view) and on sections (C). lv: left ventricle; rv: right ventricle.

FIGS. 11A-11K. Removal of Tbx5 from the ventricular cardiac conduction system reduces survival. Tbx5^{fl/fl} and Tbx5^{minKCreERT2} littermates were administered tamoxifen at 6-7 weeks of age and Tbx5 expression in the ventricular conduction system was evaluated by immunofluouresence at 10-11 weeks of age (A-J). Serial sections through the AV bundle of Tbx5^{fl/fl} mice demonstrated Tbx5 expression in the acetylcholinesterase positive, contactin-2 positive AV bundle (A-E). In contrast, Tbx5 was not detectable in the AV bundle of Tx5^{minKCreERT2} mice (F-J). Tbx5^{minKCreERT2} mice (n=22) and Tbx5^{fl/fl} littermates (n=15) were followed longitudinally following tamoxifen administration at 6-7 weeks of age (K). Kaplan-Meier survival estimates demonstrate significantly decreased survival following Tbx5 removal (* indicates p<0.05; logrank test). Original magnification: 10× in A and F, 40× in B-E and G-J. Boxed areas in A and F are shown at higher magnification in B and G. Tbx5 and contactin-2 were evaluated on serial sections; E and J represented merged images of serial sections. Nuclei are stained with hematoxylin in A, B, F, and G and DAPI in C-E and H-J.

FIGS. 12A-12H. Tbx5 expression is maintained in the atria of Tbx5^{minKCreERT2} mice. Tbx5^{fl/fl} and Tbx5^{minKCreERT2} mice were administered tamoxifen at 6-7 weeks of age and Tbx5 expression was evaluated by immunofluouresence 4-5 weeks later. Tbx5^{fl/fl} (A, C, E, G) and Tbx5^{minKCreET2} mice (B, D, F, H) demonstrated Tbx5 expression throughout the right atrium (A-B; E-F), left atrium (C-D; G-H), but not the right or left ventricles (E-H). ra: right atrium, rv: right ventricle, la: left atrium, lv: left ventricle. Nuclei are stained with DAPI (blue). Original magnification in all panels was 40×.

FIGS. 13A-13J. Conduction slowing and arrhythmias following removal of Tbx5 from the ventricular cardiac conduction system. Conduction system function in Tbx5^{fl/fl} (B, E) and Tbx5^{minKCreERT2} mice (C, F-J) was evaluated by ambulatory telemetry (B-C, G-J) and invasive EP studies (E-F). Electro-anatomical correlates of ECG and EP recordings are shown in (A) and (D), respectively. The PR and QRS intervals were prolonged during ambulatory telemetry analysis (representative recordings in B-C), and intracardiac recordings (representative recordings in E-F) demonstrated prolongation of the A-H, H_{d}, and H-V intervals. Mobitz type 2 second degree AV block (G) occurred exclusively in Tbx5^{minKCreERT2} mice. Premature ventricular contractions (H) were more common in Tbx5^{minKCreERT2} mice, and ventricular tachycardia (I and J) were observed exclusively in Tbx5^{minKCreERT2} mice. Boxed area in (I) is shown at slower scale in (J). Scale bars in all figures are 50 ms, arrows in G represent non-conducted p waves. Quantification of ECG and EP intervals are provided in Table 1.

FIGS. 14A-14B. Progressive increases in PR and QRS intervals following removal of Tbx5 from the VCS. Tbx5^{fl/fl} and Tbx5^{minKCreERT2} littermates were administered tamoxifen for 5 consecutive days beginning at 6-7 weeks of age. Telemetry recording transmitters were implanted 7 days after the first dose of tamoxifen, and recordings were made twice a week for 5 weeks. There were no changes in the PR (A) and QRS (B) intervals of Tbx5^{fl/fl} mice (blue diamonds), while the PR (A) and QRS (B) intervals of Tbx5^{minKCreERT2} mice progressively lengthened until 4-5 weeks after tamoxifen administration.

FIGS. 15A-15B. Conduction slowing following removal of Tbx5 from the VCS. Representative 6 lead surface ECG and simultaneous intracardiac recordings from the right atria (RAE) and His bundle (HBE) of Tbx5^{fl/fl} (a) and Tbx5^{minKCreERT2} littermates (b) administered tamoxifen at 6-7 weeks of age and studied 4-5 weeks after tamoxifen administration. Tbx5^{minKCreERT2} animals demonstrate prolonged PR, QRS, A-H, H_{d}, and HV intervals.

FIGS. 16A-16D. Tbx5^{minKCreET2} mice have spontaneous, sustained VT. (A) Initiation of spontaneous, monomorphic VT in a mouse with ventricular conduction system-specific deletion of Tbx5. Note the first two depolarization on the right are consistent with sinus rhythm followed by a ventricular premature depolarizations (VPD) with the same morphology as the VT that begins following two more sinus rhythm complexes. Shown are surface ECG leads I, II, III. aVR, aVL, aVF along with intracardiac recordings of the right atrial electrogram (RAE) and right ventricular electrogram (RVE). (B) Shown in this panel is initiation of the VT from panel A outlined by the box at a faster speed. Note that at the onset of the VT there does not appear to be VA disassociation but instead there is likely retrograde 1:1 ventricular-atrial conduction with fusion of the atrial and ventricular electrograms. (C)

Continuation of the same spontaneous VT shown in panel A that begins approximately 15 seconds after the end of the tracings shown in panel A. (D) Shown in this panel are the electrograms from panel C outlined by the box at a faster speed. At this time point there is now clear evidence of VA disassociation with fewer atrial electrogram (a) present on the RAE tracing compared to the number of ventricular electrograms (v) on the RVE tracing. This rhythm continued for over 2 minutes before the mouse died. P=P-wave; QRS=QRS-complex; a=atrial electrogram; v=ventricular electrogram.

FIG. 17. Tbx5^{fl/fl} control mice have only induced, non-sustained polymorphic VT. At the right side of this panel are the last 5 stimuli (S) of a drivetrain applied at a cycle length of 50-ms followed by three extrastimuli coupled at 30-ms intervals. Following delivery of the last extrastimuli non-sustained, polymorphic VT is induced that lasts approximately 420 ms. Note the presence of VA disassociation at the onset of the polymorphic VT with more ventricular electrograms (v) than atrial electrograms (a). Shown are surface ECG leads I, II, III. aVR, aVL, aVF along with intracardiac recordings of the right atrial electrogram (RAE) and right ventricular electrogram (RVE).

FIGS. 18A-18C. Normal cardiac function following loss of Tbx5 in the VCS. Cardiac function assessed by M-mode echocardiography of Tbx5^{fl/fl} controls (A) and Tbx5^{minKCreERT2} littermates (B) administered tamoxifen at 6-7 weeks of age and studied 4-5 weeks later in sinus rhythm. No functional difference between mutant and control mice could be determined. A Tbx5^{minKCreERT2} animal with episodic spontaneous ventricular tachycardia (C) demonstrated rapid recovery of ventricular function during sinus beats (asterisks) that followed tachycardic episodes.

FIG. 19A-19F. Tbx5 removal does not affect cell survival in the VCS. X-Gal (A, B, D, E) and acetylcholinesterase (AchE; C, F) stained serial sections through the AV bundle of R26R^{minKCreERT2/-}; Tbx5^{+/+} (A-C) and R26R^{minKCreERT2/+}; Tbx5^{minKCreERT2} (D-F) animals administered tamoxifen show a similar distribution of cells labeling the VCS fate map, demonstrating that Tbx5 is not required for VCS cell survival. Rectangles in A and D represent areas shown at higher magnification in B and E respectively. Original magnification in A and D is 2.5×iginal magnification in all other panels is 40×.

FIG. 20A-20T. Decreased Cx40 and Naᵥ1.5 expression in the ventricular conduction system following removal of Tbx5. The proximal and distal AV bundle were identified by acetycholinesterase activity (A, B, F, G, K, L, P, Q) and contactin-2 expression (C, D, H, I, M, N, R, S) on serial sections from Tbx5^{fl/fl} and Tbx5^{minKCreERT2} hearts. The contactin-2 positive AV bundle expressed high levels of Cx40 (C, M), Naᵥ1.5 (D, N), and Tbx5 (E, O) in Tbx5^{fl/fl} mice whereas Cx40 (H, R), Naᵥ1.5 (I, S), and Tbx5 (J, T) expression were drastically reduced in the contactin-2 positive AV bundle of Tbx5^{minKCreERT2} mice. Images shown in C, D, H, I, M, N, R, and S represent dual color immunofluoresence for Cx40 (green) and contactin-2 (red) and Naᵥ1.5 (green) and contactin-2 (red), respectively. Contactin-2 expression (red) in E, J, O, and T is from the adjacent section to that stained for Tbx5 (green); Tbx5 and contactin-2 antibodies were both raised in goat, preventing dual color immunofluouresence on the same section. Nuclei are stained with hematoxylin in A, B, F, G, K, L, P and Q and DAPI (blue) in C-E, H-J, M-O, and R-T. Rectangles in A, F, K, and P represent areas shown at higher magnification in B, G, L, and Q, respectively. Original magnification in A, F, K, and P is 10×; all other sections are shown at 40×.

FIGS. 21A-21L. Decreased Cx40 and NaV1.5 expression in midseptal, but not apical, Purkinje fibers of Tbx5^{minKCreERT2} mice. Purkinje fibers from Tbx5^{fl/fl} (A-C, G-I) and Tbx5^{minKCreERT2} (D-F, J-L) mice administered tamoxifen at 6-7 weeks of age and studied 4-5 weeks later were examined at the midseptal (A-F) and apical (G-L) level. Purkinje fibers were identified by contactin-2 (red; A, B, D, E, G, H, J, and K) or HCN-4 expression (red; C, F, I, L). Midseptal (A-C) and apical (G-I) Purkinje fibers in Tbx5^{fl/fl} mice exhibited high Cx40 (green; A, G), Naᵥ1.5 (green; B, H), and Tbx5 (green; C, I) expression. Midseptal Purkinje fibers from Tbx5^{minKCreERT2} did not express Cx40 (green; D), did not upregulate Naᵥ1.5 (green; E), and did not express Tbx5 (green; F). In contrast, apical Purkinje fibers from Tbx5^{minKCreERT2} mice expressed high levels of Cx40 (green; J), Naᵥ1.5 (green; K), and Tbx5 (green; L). Nuclei are stained with DAPI, original magnification in all panels is 40×.

FIGS. 22A-22O. Cx40 and Naᵥ1.5 expression are not altered outside the VCS in Tbx5^{minKCreERT2} mice. Atrial expression of Naᵥ1.5 was examined in the right (A-B) and left (C-D) atria of Tbx5^{fl/fl} (A,C) and Tbx5^{minKCreERT2} (B,D) mice by immunofluoresence and Western Blotting of whole atria (E). No differences were detected in atrial Naᵥ1.5 expression. Atrial expression of Cx40 was examined in the right (F-G) and left (H-I) atria of Tbx5^{fl/fl} and Tbx5^{minKCreERT2} mice by immunofluoresence and Western Blotting of whole atria (J). No differences were detected in atrial Cx40 expression. Ventricular expression of Naᵥ1.5 was examined in the right (K-L) and left (M-N) ventricles of Tbx5^{fl/fl} (K, M) and Tbx5^{minKCreERT2} (L, N) mice by immunofluoresence and Western Blotting of whole ventricles (O). No differences were detected in Naᵥ1.5 expression. Graphs in E, J, Orepresent mean+/−SD; representative blots for Naᵥ1.5 and Cx40 (Tbx5^{fl/fl} on the left, Tbx5^{minKCreERT2} on the right) are shown. Hsp90 blots demonstrate equal loading of protein.

FIGS. 23A-23I. Tbx5 directly regulates an enhancer downstream of Scn5a. Bioinformatic identification of a candidate enhancer (highlighted yellow) downstream of Scn5a (A). The WT candidate enhancer demonstrated robust Tbx5-mediated activation in dual luciferase reporter assays in HEK-293T cells (B). Luciferase activity was blunted by single mutation of any of 3 conserved T-box elements (TBE1, 2, or 3 mut) and eliminated by mutation of all 3 T-box elements (TBE123 mut). The WT enhancer reproducibly drove lacZ expression from a minimal promoter in the VCS of transient transgenic embryos analyzed at E13.5. (C): representative posterior view of transgenic heart stained with X-Gal; (D): sagittal section of transgenic heart stained with X-Gal. Boxed region (from D) is presented at higher magnification (E), demonstrating X-gal expression in the developing AV bundle (arrowhead) and bundle branches (arrow). Mutation of the T-box elements in the enhancer resulted in blunted and regionally variable expression (F): posterior view of representative transgenic heart demonstrating weak, non CCS lacZ expression (G): saggital section of a more robustly stained heart demonstrates ectopic expression in the endocardial cushions and compact myocardium. Boxed region (from G) presented at higher magnification (H) demonstrates absence of X-gal expression in the AV bundle (arrowhead) and bundle branches (arrow). Model for the role of Tbx5 in driving fast conduction in the VCS via direct regulation of SCN5A and Cx40 (I). * represents p<0.05 vs. all other groups; n>3; mean±SEM . Original magnification in D and G: 4×; E and H: 40×.

FIG. 24. Evaluation of all WT and TBE123 mutant enhancers at E13.5. 16 transgenic embryos were genotypically positive for the WT enhancer. 13 (shown) demonstrated LacZ expression. All embryos with LacZ expression demonstrated VCS expression. 12 transgenic embryos were genotypically positive for the TBE123 mutant enhancer. 11 (shown) demonstrated LacZ expression. 5 (bottom row) demonstrated minimal cardiac expression. Of the 6 embryos with significant cardiac expression, 4 were sectioned, and only 1 showed VCS expression; based on this the inventors conclude that a maximum of 3 mutant embryos have LacZ expression in the VCS. Embryos are arranged in descending order of staining intensity. Boxed embryos were sectioned. Sections are shown at 10× original magnification.

FIGS. 25A-25F. GWAS on CCS function and cardiac ChIP-Seq data predict CCS enhancers. Genome Browser view of chr 3: 38,359,623-38,915,287 demonstrating that SNPs tagged in GWAS studies (green) fall into two distinct LD blocks near the SCN5A/10A locus (A). The mouse equivalent of the region depicted above is shown (chr 9: 119,266,571-119,699,765) along with ChIP-Seq peaks for cardiac transcription factors in HL-1 cells (He, et al., 2011) (B). Two regions with overlap of multiple ChIP Seq peaks (highlighted yellow in B) were tested for CCS enhancer activity in vivo. Both the 5′ enhancer (C-D) and 3′ enhancer (E-F) demonstrated cardiac enhancer activity, with strongest expression in the developing CCS.

FIGS. 26A-26J. 5′ and 3′ enhancers are required for cardiac expression at the Scn5a locus. Enhancers (highlighted yellow in A and red in B) were identified by cardiac ChIP-Seq results (A). A BAC, RP23-198L19, was modified such that LacZ was inserted into the endogenous Scn5A translational start site (B). Transient transgenic embryos harboring the wild-type BAC (C, G), a BAC with the 5′ enhancer deleted (D, H), the 3′ enhancer deleted (E, I), or both enhancers deleted (F, J) were evaluated at E13.5. The wild-type enhancer (C, G) demonstrated robust cardiac expression, while single removal of either enhancer (D, H, E, I) blunted cardiac expression. Removal of both enhancers ablated cardiac LacZ expression (F, J). Arrows indicate the proximal and distal AV bundle; black arrows highlight expression in the indicated region, while red arrows highlight lack of expression.

FIG. 27A-27D. Model for a potential role for Tbx5 and Tbx3 in driving nodal VCS identity in the atrioventricular conduction system. The AV node endogenously possesses high Tbx3 and low Tbx5 activities, while the VCS has high Tbx5 and low Tbx3 activities (A). This drives slow conduction in the node and specialized fast conduction in the VCS. This model predicts that removal of Tbx5 (B) or overexpression of Tbx3 (C) in the VCS may uncover a slow conducting, nodal phenotype. Removal of both Tbx5 and Tbx3 (D) may transform the AVB into ventricular myocardium.

FIG. 28. List of genes significantly upregulated in E10.5 minKGFP^{high} cells.

FIG. 29. List of genes upregulated in E10.5 minKGFP^{low} cells.

FIG. 30. Procedure of embryonic stem cell (ES cell) differentiation to cardiomyocytes.

FIG. 31. Procedure of cardiac mesoderm segregation for cardiac conduction system induction.

FIGS. 32A-E. Tbx3 ES cell line was successfully differentiated to beating cardiomyocytes (A, >80%). The Tbx3-overexpression induction during different periods by doxycycline showed phenotypical differences (B-E).

FIGS. 33A-C. (A) By Tbx3 over-expression induced by doxycycline, the expression level of Tbx3, slow conduction marker, was up-regulated. (B) By Tbx3 over-expression induced by doxycycline, the expression level of MLC-2a, myocardium marker, was up-regulated. (C) By Tbx3 over-expression induced by doxycycline, the expression level of SCN5a, conduction system marker, was down-regulated.

FIGS. 34A-E. Tbx5 ES cell line was successfully differentiated to beating cardiomyocytes (A, >80%). The Tbx5-overexpression induction during different periods by doxycycline showed phenotypical differences (B-E).

FIGS. 35A-C. (A) By Tbx3 over-expression induced by doxycycline, the expression level of Tbx5, fast conduction system marker, was up-regulated. (B) By Tbx5 over-expression induced by doxycycline, the expression level of Tbx3 was up-regulated. (C) By Tbx5 over-expression induced by doxycycline, the expression level of HCN4, sa conduction system marker, was up-regulated.

### DESCRIPTION OF ILLUSTRATIVE EMBODIMENTS

### A. THE CARDIAC CONDUCTION SYSTEM

1. Function and Anatomy

The cardiac conduction system (CCS) consists of specialized cardiomyocytes that initiate and propagate electrical impulses to allow coordinated contraction of the myocardium and ultimately efficient circulation of blood throughout the organism. In the adult heart, electrical impulses are generated at the sino-atrial (SA) node, propagated through fast-conducting atrial myocardium, and delayed at the atrio-ventricular (AV) node. This delay allows for atrial contraction to complete prior to the onset of ventricular contraction. From the AV node, the electrical impulse is rapidly propagated through the ventricular conduction system (VCS), which consists of the AV (His) bundle and bundle branches and the peripheral Purkinje network, which directly transmits the electrical impulse to working ventricular myocardium. This arrangement permits the apex to base activation sequence necessary for coordinated ventricular contraction and efficient ejection of blood from the ventricles into the outflow tracts. Disorders of the CCS are common, carry significant morbidity, and are poorly understood from a molecular perspective.

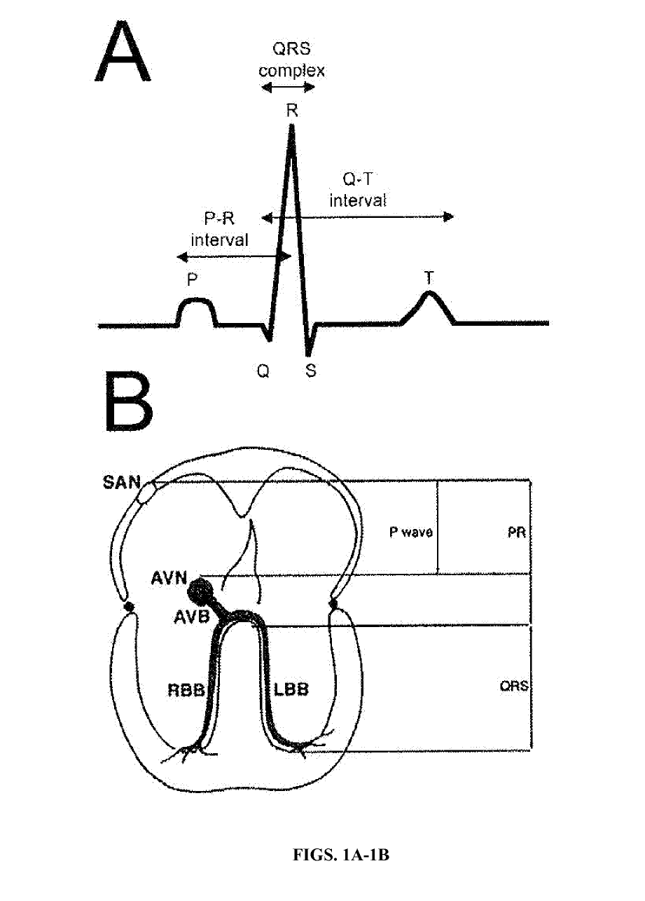

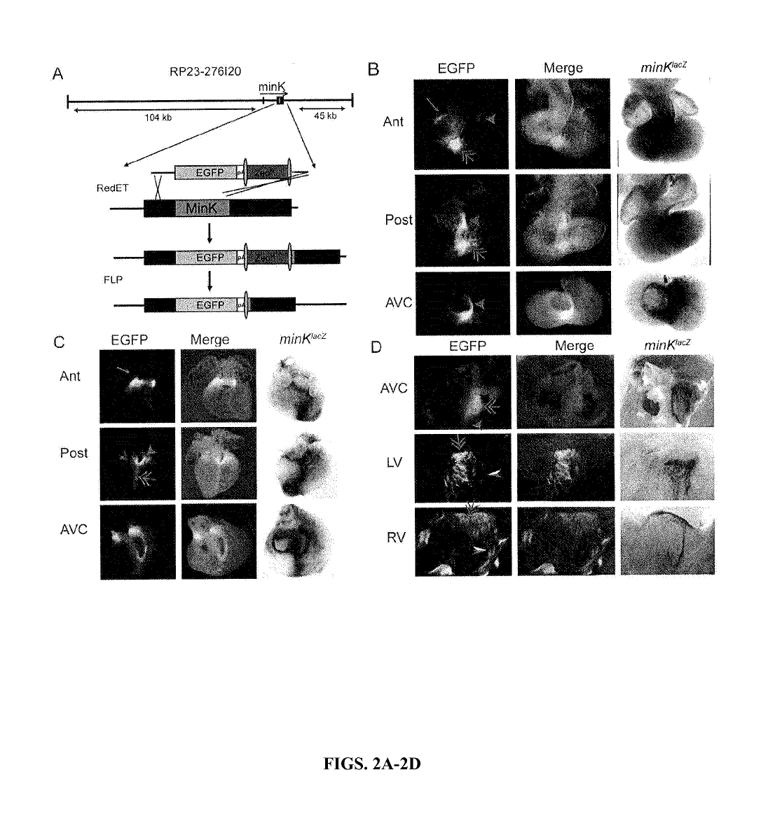

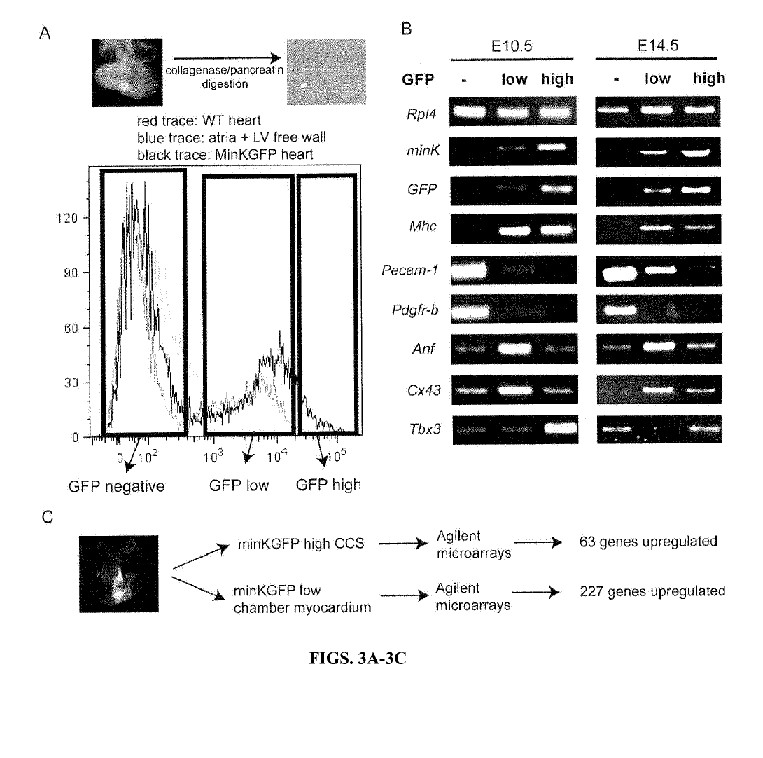

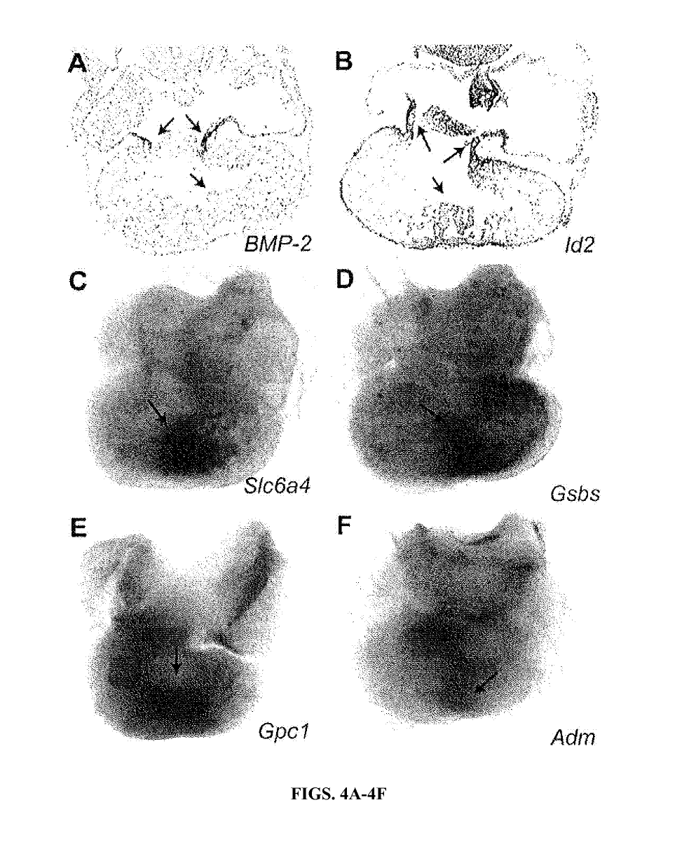

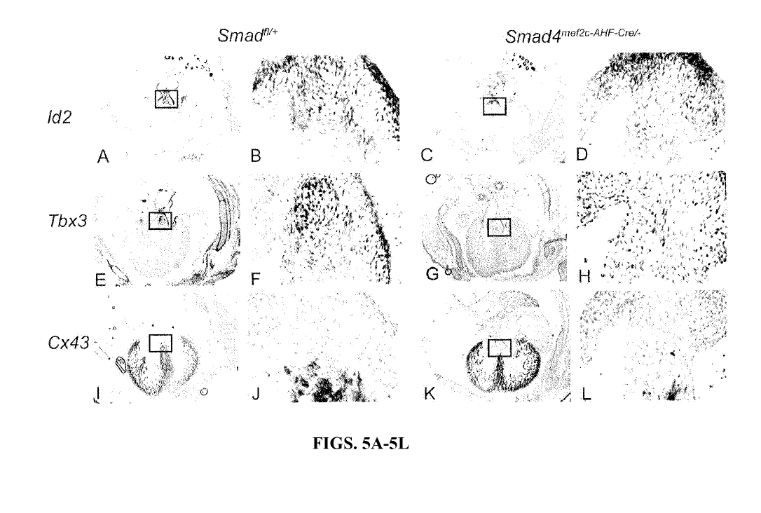

Understanding the transcriptional networks that are required for CCS development and maintenance requires assessment of CCS function. ECG (electrocardiogram) measures have been a valuable tool for decades for inferring proper function of the CCS. The routine 12 lead ECG allows analysis of the electrical vectors across the precordium. The P wave and QRS complex reflect atrial and ventricular depolarization, respectively. The broad shaped T wave represents ventricular repolarization. Directly obtainable measures from the routine ECG include the heart rate, PR interval, QRS duration, and QT interval (FIGS. 1A-1B). These measures are functional readouts of CCS and myocardial function. Thus, heart rate and variability are indicative of SA node function, the PR interval encompasses the AVN and proximal VCS function, the QRS duration measures conduction through the VCS and ventricular myocardium, and the QT interval includes repolarization of the ventricular myocardium. Since these intervals are so easily and rapidly determined for large numbers of individuals, ECG intervals have clear statistical norms; values diverging from these norms imply dysfunction at the level of the associated CCS or myocardial structure.

Knowledge of the genetic contributions to CCS dysfunction has until recently relied largely on heritable monogenic diseases. Studies of families with heritable monogenic CCS disease, as well as mouse models of these diseases, have identified a limited number of genes essential to maintaining cardiac rhythm, most of which encode ion channels and their interacting partners (Priori, 2010). In contrast to the single gene Mendelian disorders that provide highly useful information in families, genome-wide association studies (GWAS) can discern genetic contribution across populations. Although GWAS are very powerful to identify genetic regions that contribute to the phenotype of interest, by design, these studies identify common variants in the population. To have achieved such prevalence in the population, these DNA variants typically only confer relatively modest effects. The ubiquity and established norms of ECG measures worldwide has provided an excellent platform for investigating the genetic contributions to variations in CCS function. Several groups have exploited this platform and recently reported the results of genome-wide association studies for ECG intervals, including heart rate (Cho, et al., 2009; Holm, et al., 2010; Newton-Cheh, et al., 2007), PR interval (Holm, et al., 2010; Newton-Cheh, et al., 2010; Pfeufer, et al., 2010), QRS interval (Holm, et al., 2010; Sotoodehnia, et al., 2010; Chambers, et al., 2010), and QT duration (Holm, et al., 2010; Chambers, et al., 2010; Arking, et al., 2009; Newton-Cheh, et al., 2009; Pfeufer, et al., 2009). These results (Table 1) were notable for the remarkable number of genes implicated in heart development, as well as developmental processes more generally (Arnols, et al., 2011). While genes near some of the loci have known roles in CCS development, the biology underlying many of these associations is opaque.

TABLE 1
Genes Near Loci Associated with Variation in ECG Intervals in Genome Wide
Association Studies.

| HR | P-R | QRS | | Q-T |
|---|---|---|---|---|
| MYH6¹ | SCN10A^{1,2,3,10} | SCN10A^{1,3,4} | SETBP1⁴ | SCNSA^{1,3,5,6} |
| GJA1⁹ | SCN5A2 | SCN5A⁴ | TKT-PRKCD- | C6orf204-SCL35F1- |
| CD46-CD34⁹ | Tbx5^{1,2} | Tbx5^{1,4} | CACNA1D⁴ | PLN-BRD7P3^{1,3,5,6,7} |
| | Tbx3² | Tbx3⁴ | CR1M1⁴ | Tbx5¹ |
| | CAV1^{1,2} | C6orf204-SCL35F1- | C1orf185-RNF11- | NOS1AP^{1,3,5,6,7,8} |
| | ARGHAP24^{1,2} | PLN-BRD7P3⁴ | CDKN2C-FAF1⁴ | ATP1B1^{1,3,6} |
| | MEIS1² | CDKN1A^{1,4} | PRKCA⁴ | KCNH2^{1,3,5,6} |
| | WNT11² | NFIA⁴ | IGFBP3⁴ | LITAF^{1,3,5,6} |
| | SOX5² | HAND1-SAP30L⁴ | CASQ2⁴ | KCNJ2^{1,3,6} |
| | NKX2-5² | SIPA1L1⁴ | KLF12⁴ | KCNQ1^{1,5,6} |
| | | Tbx20⁴ | LRG1-SLC25A26⁴ | CNOT1⁵ |
| | | VTI1A⁴ | DKK1⁴ | RNF207^{1,5,6} |
| | | GOSR2⁴ | HEATR5B-STRN⁴ | KCNE1^{1,5} |
| | | | | LIG3^{5,6} |
| | | | | NDRG4-GINS3^{5,6} |

Loci listed in bold have been associated with more than one ECG interval.
Underlined loci are linked to developmental processes (see text for details).
Superscript numbers reference studies reporting significant genome wide association with the indicated locus as follows:
¹Holm et al., 2010
²Pfeufer et al., 2010
³Chambers et al., 2010
⁴Sotodoohenia et al., 2010
⁵Newton-Cheh et al., 2009
⁶Pfeufer et al., 2009
⁷Nolte et al., 2009
⁸Arking et al., 2006
⁹Cho et al., 2009
¹⁰Denny et al, 2010.

Despite the clinical consequences of conduction system dysfunction, relatively little is known about the molecular networks underlying the development of the conduction system and whether the same or different networks later ensure function of the mature conduction system. In addition to the association between loci identified in GWAS on CCS function and development, developmental bases have been described for numerous disorders of the conduction system, including life-threatening ventricular arrhythmias, highlighting the relevance of conduction system development to human disease (Boukens, et al., 2009). Furthermore, understanding the developmental and transcriptional cues guiding the formation of the conduction system as well as those that are essential to maintain function of the mature CCS will be essential to future efforts to use regenerative medicine to treat diseases of the conduction system (Chien, et al., 2008). Key goals in advancing the understanding of the molecular networks that function in the CCS thus clearly include achieving a greater understanding of the networks that function during CCS development, as well as the potential role of developmentally important genes in maintaining function of the mature CCS.

2. Development of the Conduction System

The cardiac conduction system develops in concert with the embryonic heart tube. In the early heart tube, the “primary myocardium” is weakly contractile with a poorly developed sarcoplasmic reticulum, possesses intrinsic automaticity, proliferates slowly and is slowly conducting (Moorman, et al., 2003). As the heart tube loops, atrial and ventricular chamber myocardium differentiates and acquires a highly proliferative, fast conducting, strongly contractile phenotype, while a primary myocardial phenotype is retained in the AV canal, primary (interventricular ring) and outflow tract during early development. Maturation of chamber myocardium is associated with the expression of molecular markers such as Anf and Cx43 and involves core cardiogenic transcription factors with broad expression domains, including Gata-4, Nkx2-5, and Tbx5 (Moorman, et al., 2003). In addition to their broader roles throughout the heart, Nkx2-5 and Tbx5 have specific roles in the development of the conduction system, discussed below.

Given that the central conduction system shares many features with primary myocardium, including high automaticity, low contractility and a low proliferative index, the current paradigm in the field is that conduction system precursors, which have been shown to express a number of transcriptional repressors, including Tbx2, Tbx3, and Id2 (Christoffels, et al., 2009; Habets, et al., 2002; Hoogaars, et al., 2004; Moskowitz, et al., 2007), are prevented from differentiating into chamber myocardium, preserving elements of their primary, or primitive, phenotype. There are notable differences between primary myocardium and the CCS, however, and the transcriptional networks that govern this transition are not well understood.

3. Development of the AV Node

The AV node delays the electrical impulse between the atria and ventricles, allowing atrial contraction to complete prior to the onset of ventricular contraction. The mature AV node is located in the floor of the right atrium and is in communication with the ventricles via the AV bundle. Developmentally, AV nodal function (i.e. a delay in the propagation of the electrical impulse from the atria to the ventricles) is provided by the AV canal from at least E9.5 onwards (Valderrabano, et al., 2006). The transcriptional repressors Tbx2 and Tbx3 operate in a complex feed-forward loop with BMP signaling to induce formation of the AV canal (Singh, et al., 2012). Classical morphologic as well as more recent molecular evidence supports the hypothesis that the AV canal is the precursor to the AV node (Viragh, et al., 1977a; Viragh, et al., 1977b; Wessels, et al., 1992; Davis, et al., 2001; Horsthuis, et al., 2009). Most notably, cells derived from the embryonic AV-canal, marked by a Tbx2-Cre transgene, later populate the mature AV node, in addition to the AV valves and the left ventricle (Aanhaanen, et al., 2009). Dominant mutations in TBX5 and NKX2-5, which also play important roles in the development of the AV bundle and bundle branches, lead to AV block in humans (Basson, et al., 1997; Li, et al., 1997; Schott, et al., 1998) and mice (Bruneau, et al., 2001; Jay, et al., 2004), as well as patterning defects in the AV node, thus implicating these core cardiogenic transcription factors in the development and/or maintenance of the AV node. Additional molecular players potentially involved in the development and function of the AV node have been identified in a recently published study analyzing genes upregulated in the developing AV node, marked by a Tbx3GFP transgene expressed specifically in the developing and mature AV node, but excluded from the AV bundle, bundle branches, and chamber myocardium (Horsthuis, et al., 2009).

4. Development of the AV Bundle and Bundle Branches

The AV (His) bundle, bundle branches, and peripheral Purkinje network constitute the ventricular conduction system, and are responsible for rapidly propagating the impulse from the AVN across the AV junction to the ventricular apex. The AV bundle and bundle branches share some features with the AV node, including a poorly contractile, slowly proliferating phenotype, and expression of Tbx3 but not Cx43, but also have significant differences, most notably their fast conducting phenotype. Furthermore, they are derived from distinct cell populations, as revealed by the finding that cells that have expressed Tbx2, which constitute the entirety of the AV canal, later populate the AV node, but not the AV bundle or bundle branches (Aanhaanen, et al., 2009); conversely, cells derived from the Mef2cCre lineage (Verzi, et al., 2005) populate the AV bundle and bundle branches, but are excluded from the AV node (Aanhaanen, et al., 2010). The central and peripheral ventricular conduction system also arise from distinct developmental lineages, as the AV bundle and proximal bundle branches are believed to derive from the myocardium of the primary ring, while the peripheral Purkinje network is believed to derive from trabecular myocardium (Christoffels, et al., 2009; Miquerol, et al., 2011). Segregation of these lineages may occur as early as E8, when Tbx2 expression is initiated in the cardiac crescent. Morphologically, cells of the forming AV bundle become distinguishable between E10 and E11 (Viragh, et al., 1977b), and electrophysiological evidence demonstrates a functional fast ventricular conduction system in the mouse by E10.5. Apex to base excitation has been observed in embryonic hearts from E10.5 onwards, in contrast to the immature base to apex propagation at E9.5 (Valderrabano, et al., 2006; Rentschler, et al., 2001).

The AV bundle and proximal bundle branches are believed to develop from a specialized ring of cells that are visible at E10.5 in the mouse and link the tip of the nascent interventricular septum with the right AV canal. These cells constitute the primary/interventricular ring and can be identified by a unique histologic and ultrascopic appearance (Viragh, et al., 1977b; Wenink, 1976), as well as by expression of minK^{LacZ} (Kupershmidt, et al., 1999; Kondo, et al., 2003) and CCS-LacZ (Rentschler, et al., 2001) in the mouse, HNK-1 in the rat (Ikeda, et al., 1990), NF in the rabbit (Gorza, et al., 1989), and G1N2 (Wessels, et al., 1992) in human fetal hearts. An emerging paradigm for VCS specification suggests that specific transcriptional activators, such as Tbx5, drive expression of genes required for fast VCS function, and that transcriptional repressors, such as Tbx3 and Id2, prevent differentiation into chamber myocardium (Moskowitz, et al., 2007; Bakker, et al., 2008). The molecular networks that act upstream of these transcription factors, and more broadly in early specification of the conduction system, are not well understood.

5. Tbx5 and the Ventricular Conduction System

Dominant mutations in TBX5 cause Holt-Oram syndrome (Basson, et al., 1997; Li, et al., 1997; Basson, et al., 1994), which presents with upper limb malformations and a high incidence of congenital heart malformations. These most commonly comprise atrial septal defects, although a wide range of congenital heart defects have been reported, including atrial and ventricular septal defects, as well as more complex malformations such as Tetralogy of Fallot (Basson, et al., 1994). Furthermore, conduction system abnormalities, particularly progressive AV block, are a common feature of Holt-Oram syndrome, and electrophysiological abnormalities have been reported in the absence of structural abnormalities (Basson, et al., 1994; McDermott, et al., 2005). These findings, as well as the consistent identification of TBX5 in GWAS on VCS function strongly suggest a role for Tbx5 within the conduction system independent of its role in structural morphogenesis of the heart.

The role of Tbx5 within the conduction system has been studied extensively in mouse models. Deletion of a single copy of Tbx5 in mice recapitulates many features of human Holt-Oram syndrome, including both structural and electrophysiological defects (Bruneau, et al., 2001). Specifically, adult Tbx5^{+/-} mice demonstrate prolonged PR intervals and some but not all studies demonstrate wide QRS intervals, indicative of defects in conduction from the atria through the proximal bundle branches and through the bundle branches and Purkinje fibers, respectively (Bruneau, et al., 2001; Moskowitz, et al., 2004). These electrophysiological abnormalities are associated with abnormal morphology of the conduction system, as assessed by minK^{LacZ} expression. Adult Tbx5^{+/-} animals fail to consolidate rings of minK^{LacZ} positive tissue at the AV junction, present in juvenile animals, into a well-formed AV node, and neonatal and adult Tbx5^{+/-} animals have greatly diminished minK^{LacZ} staining in the right ventricle, suggesting agenesis or hypoplasia of the right bundle branch. This correlates with the presence of right bundle branch block in adults and suggests a role for Tbx5 in the development and maturation of the ventricular conduction system. While Tbx5 clearly plays a key role in development and maturation of the CCS, the molecular networks controlled by Tbx5 are only beginning to be understood, and it is completely unknown whether Tbx5 plays an ongoing role in the mature VCS.

6. Transcriptional Control of Mature CCS Function

Little is known regarding the transcription factors that are required to maintain specialized CCS phenotypes in the mature heart. Postnatal knockouts have been reported for only a limited number of cardiac transcription factors with relevance to CCS function, and analysis of these results with regards to their specific role in the CCS is complicated by the fact none of the knockouts were CCS-specific, but rather encompassed all cardiomyocytes or all cells within the animal. Tamoxifen-inducible deletion of Nkx2-5 at 2 weeks of age resulted in slow, progressive development of conduction and contraction defects, with 1^{st} degree AV block becoming apparent 7 weeks after tamoxifen administration (Takeda, et al., 2009). This effect was markedly less severe than the rapid effects following perinatal removal of Nkx2-5 (Briggs, et al., 2008), but nonetheless demonstrates an ongoing role for Nkx2-5 in the mature heart. Removal of Tbx20 at 6-8 weeks of age resulted in the rapid development of arrhythmias and the onset of severe cardiomyopathy (Shen, et al., 2011), demonstrating an essential role for Tbx20 in the adult heart. During development, Tbx20 plays a key role in chamber differentiation and in restricting Tbx2 expression to the AV canal (Greulich, et al., 2011). Notably, Tbx20 is excluded from the AV bundle during development (Bakker, et al., 2008), and its adult expression pattern in the CCS has not been reported. This suggests that the reported arrhythmias may be secondary to effects in the ventricular myocardium. Tamoxifen-inducible deletion of Tbx3 at 8-12 weeks of age resulted in a transient increase in 2^{nd} degree AV block, demonstrating a role for Tbx3 in the adult heart (Frank, et al., 2012). Analysis of the extent of that role is complicated, however, by the finding that Tbx3 expression was reduced, but not eliminated following tamoxifen induction of Cre activity. These reports demonstrate ongoing roles for developmental transcriptional factors in the mature heart, but also point to the limited extent of the knowledge of these networks and the need for efficient, CCS-specific Cre drivers.

7. Tbx5 and Tbx3

Various embodiments relate to increasing the amount of Tbx5 or Tbx3 in a cardiomyoctye whereby the cardiomycyte is converted to a fast conducting cardiomyocyte or a slow conducting nodal cell, respectively. The inventors demonstrated that Tbx5 plays a crucial role in maintaining fast conduction in the VCS by driving a molecular network that includes direct regulation of Cx40 and Scn5a. Tbx3 represses Cx40 and Scn5a in vivo and is capable of activating a functional and molecular nodal phenotype (Hoogaars, et al., 2007; Bakker, et al., 2012). Tbx3 is expressed throughout the AVCS (Hoogaars, et al., 2004; Aanhaanen, et al., 2010), although Tbx5 activity appears to be dominant in the VCS. Cumulatively these results indicate that Tbx5 and Tbx3 are key determinants of regional identity in the AVCS.

The inventors further demonstrated that loss of Tbx5 results in a loss of fast conduction in the VCS. Thus, in the absence of Tbx5, Tbx3 activities will predominate, which transforms the AVB into a nodal-like structure. This may be confirmed by directly measuring ionic currents in VCS cells in the presence or absence of Tbx5 to determine if loss of Tbx5 results in a transformation to a nodal phenotype at the single cell level and determining if the AVB adopts a nodal molecular profile in the absence of Tbx5. Molecular evaluation can be achieved using established markers of the AVN, such as Cx30.2 (Kreuzberg, et al., 2006), ion channel profiles of the AVN identified by microdissection (Gaborit, et al., 2007; Greener, et al., 2011; Marrioneau, et al., 2005; Greener, et al., 2009) as well as published expression profiles of BACTbx3-EGFP hearts that express EGFP exclusively in the AVN (Horsthuis, et al., 2009). The overexpression of Tbx3 in the AVB will result in a similar phenotype to removal of Tbx5. One approach to demonstrating this is to cross a Cre-conditional Tbx3 overexpression line (Hoogaars, et al., 2007) with minKCreER^{T2} mice. Finally, the loss of both Tbx5 and Tbx3 from the VCS will cause a transformation to a working myocardial phenotype. This can be evaluated in Tbx3 floxed mice (Frank, et al., 2012). Thus, Tbx5 and Tbx3 are key determinants of fast and slow conduction in the CCS.

It had not been previously possible to evaluate the specific role for Tbx5 within the conduction system because of its broad cardiac expression in the adult heart, its requirement during cardiac development, and the structural heart defects that frequently are associated with Tbx5 haploinsufficiency (Bruneau, et al., 2001; Basson, et al., 1994). By selectively removing Tbx5 from the adult VCS, the inventors identified a previously unknown role for Tbx5 in the mature VCS of structurally normal hearts. Specifically, removal of Tbx5 from the mature VCS resulted in a significant increase in mortality accompanied by arrhythmias, including ventricular tachycardia, and a dramatic slowing of conduction through the VCS. Slowed VCS conduction was manifest as AV block with dramatic increases in the H_{d} and H-V intervals as well as a prolonged QRS interval, demonstrating interventricular conduction delay. Loss of fast conduction in the VCS was not secondary to a loss of contractile function or loss of VCS cells, but rather was associated with reductions in Cx40 and Naᵥ1.5 expression in the VCS. Tbx5 directly regulates Naᵥ1.5 expression via an enhancer downstream of the Scn5a locus that possesses T-box element dependent VCS-specific expression in vivo. The results establish the first transcriptional pathway required for function of the mature VCS and establish Tbx5^{minKCreERT2} mice as a model for the pathogenesis of ventricular conduction system disease.

The inventors identified a novel role for Tbx5, a congenital heart disease (CHD) gene, in the adult heart, implicating a developmental transcription factor in the molecular pathways that coordinate function in the mature heart. In elderly patients with congenital heart disease in the CONCOR registry, arrhythmias have been reported in over 50% of patients (van der Bom, et al., 2011). Although some portion of this is likely secondary to factors such as structural abnormalities and surgical complications, the study demonstrates that CHD genes including Tbx5 are not limited to coordinating cardiac morphogenesis but also may play a broader role in coordinating mature heart function. This paradigm may be particularly relevant to study of the conduction system in light of the genetic variation near a large number of developmental genes identified in GWAS on PR and QRS intervals (Arnolds, et al., 2011).

Tbx3 is closely related to Tbx5, and they frequently antagonistically regulate gene expression (Greulich, et al., 2011). Genes repressed by Tbx3, particularly in the atria where Tbx5 expression is high, are thus potential Tbx5 targets. The potential utility of this intellectual framework is highlighted by the finding that Cx40 and Scn5a are downregulated following Tbx3 overexpression (Hoogaars, et al., 2007; Bakker, et al., 2012). Of the numerous genes affected by Tbx3 overexpression (Hoogaars, et al., 2007; Bakker, et al., 2012), Kcnk3/TASK-1, Ryr2, and Kcnj2 (Kv1.2), are notable for their known functional roles in the ventricular conduction system. Tbx3 is a potent transcriptional repressor capable of repressing Nav1.5 (Hoogaars, et al., 2007), which may underlie exclusion of Nav1.5 from the nodes.

### B. EMBRYONIC STEM CELLS AND INDUCED PLURIPOTENT STEM CELLS

The cardiomyocytes employed in various embodiments disclosed herein may be differentiated from a pluripotent cell such as an embryonic cell (ES), embryonic germ cells (EG), or an induced pluripotent stem cell (iPS). A “pluripotent cell” or “pluripotent stem cell” is a cell that has the capacity to differentiate into essentially any fetal or adult cell type. Thomson et al. (Proc. Natl. Acad. Sci. USA 92: 7844, 1995) successfully culture embryonic stem cells from primates, using rhesus monkeys and marmosets as a model, and subsequently derived human embryonic stem (hES) cell lines from human blastocysts (Science 282: 114, 1998). Gearhart and coworkers derived human embryonic germ (hEG) cell lines from fetal gonadal tissue (Shamblott et al., Proc. Natl. Acad. Sci. USA 95: 13726, 1998).

Both embryonic stem cells and embryonic germ cells can proliferate in vitro without differentiating. Culture environments that allow for continuous proliferation of pluripotent stem cells are described, for example, in WO 01/51616, which is incorporated herein by reference.

Induced pluripotent stem (iPS) cells are reprogrammed somatic cells that exhibit stem cell pluripotency and express embryonic markers (Takahashi et al. Cell, 131(5):861-872, 2007; Takahashi et al. Nat. Protoc., 2(12):3081-3089, 2007). Methods of producing iPS cells are known in the art, and essentially any appropriate method of reprogramming a somatic cell may be used to produce a pluripotent stem cell for use in a method disclosed herein. Exemplary methods of generating iPS cells may include, for example, methods disclosed by Yamanaka (U.S. Pat. Nos. 8,048,999 and 8,058,065), Thomson (US 2008/0233610), and Daley (US 2009/0004163), all of which are incorporated herein by reference in their entirety.

Conditions have been described for differentiating stem cells into a variety of tissues, including cardiomyocytes. For example, U.S. Pat. No. 7,763,464, which is incorporated herein by reference, describes methods of differentiating embryonic stem (ES) cells into cardiomyocyte lineage cells. These methods include, for example, a) obtaining hES cells and forming an embryoid body in a cell culture; b) culturing the embryoid body from a) in the cell culture with activin, transforming growth factor (TGF), and insulin-like growth factor (IGF), c) subsequently adding bone morphogenic protein (BMP), fibroblast growth factor (FGF), insulin, and platelet-derived growth factor (PDGF); to the cell culture from b) and then d) adding a nucleotide analog that affects DNA methylation to the cell culture of c) thereby differentiating hES cells into cardiomyocytes.

As a futher example, U.S. 2011/0104122, which is incorporated herein by reference, discloses methods for producing cardiomyocytes and/or cardiac progenitor cellsfrom iPS cells or embryonic stem (ES) cells. These methods generally involve culturing an iPS cell or ES cell, which has been differentiated into a mesoderm cell, in the presence of cyclosporine-A.

### C. GENETIC ALTERATION OF CELLS

In certain embodiments Tbx5 or Tbx3 is overexpressed in a cardiomyocyte to convert the cariomyocyte to a fast conducting cardiomyocyte or a slow conducting nodal cell, respectively. In certain aspects, the overexpression is achieved by introducing a nucleic acid sequence encoding Tbx5 or Tbx3 into the cell.

The cells may be genetically altered by transfection or transduction with a suitable vector, homologous recombination, or other appropriate technique, so that they express Tbx5 or Tbx3, typically under a heterologous promoter that increases their expression beyond what occurs under their endogenous promoter. Methods for delivering nucleic acids to cells include, but are not limited to, the use of vectors, such as viruses and plasmids, lipofection, electropolation, electroporation, microinjection, and the like. Examples of viral vectors, which are replication-deficient, include, but are not limited to, retrovirus vectors, lentivirus vectors, adenovirus vectors, adeno-associated virus vectors. Examples of commercially available viral vectors are: pMXs, pMYs and pMZs (Cosmo Bio, Japan), which are deficient in gag; retro-XQ vectors (Clontech, the Netherlands), pLenti6/Ubc vectors (Invitrogen, USA).

The genes may be operably linked to a regulatory sequence(s), such as promoter and enhancer, so that they are capable of being expressed. The vectors may further comprise a positive selectable marker(s), such as a drug resistance gene(s) (e.g., puromycin resistance gene, neomycin resistance gene, ampicillin resistance gene, hygromycin resistance gene, or the like), a negative selectable marker(s) (e.g., diphtheria toxin A fragment gene, thymidine kinase gene, or the like), an internal ribosome entry site (IRES), a terminator, an origin of replication, etc. The cells may then be assessed for fast conducting cardiomyocyte or a slow conducting nodal cell phenotypes.

### D. USE OF FAST CONDUCTING MYOCYTES AND SLOW CONDUCTING NODAL CELLS

The fast conducting cardiomyocytes and slow conducting nodal cells disclosed herein can be used for a number of important research, development, and commercial purposes. For example, the cells can be used to prepare cDNA libraries specific for the fast conducting cardiomyocytes or the slow conducting nodal cells. Additionally, The cells can also be used to prepare antibodies that are specific for markers of these cell types. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, which are incorporated herein by reference. By positively selecting using the fast conducting cardiomyocytes and/or slow conducting nodal cells, and negatively selecting using cells bearing more broadly distributed antigens the desired specificity can be obtained. The antibodies in turn can be used to identify or rescue heart cells of a desired phenotype from a mixed cell population.

The cells are also of interest in identifying expression patterns of transcripts and newly synthesized proteins that are characteristic for fast conducting cardiomyocytes and/or slow conducting nodal cells.

The fast conducting cardiomyocytes and slow conducting nodal cells can be used to screen for factors (such as solvents, small molecule drugs, peptides, oligonucleotides) or environmental conditions (such as culture conditions or manipulation) that affect the characteristics of these cells. The cardiac conduction system (CCS) is essential to ensure a regular heartbeat, coordinated contraction of the heart, and ultimately efficient circulation of blood throughout the organism. Thus, the methods and cells disclosed herein provide a valuable resource for identifying causes and treatments for conduction system disease. Accordingly, the screening applications described herein can be used in the testing of pharmaceutical compounds for their effect on CCS diseases. Screening may be done either because the compound is designed to have a pharmacological effect on the cells, or because a compound designed to have effects elsewhere may have unintended side effects on cells of this tissue type.

Assessment of the activity of candidate pharmaceutical compounds generally involves combining the cells with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change.

Effect of cell function can be assessed using any standard assay to observe phenotype or activity of cardiac cells. A number of assays are described in detail in the Examples below. Where an effect is observed, the concentration of the compound can be titrated to determine the median effective dose (ED₅₀).

The fast conducting cardiomyocytes and slow conducting nodal cells may also be used therapeutically to enhance tissue maintenance or repair in the CCS for any perceived need, such as a birth defect, the effect of a disease condition, or the result of trauma.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the desired cells are still present. Suitability can also be determined by assessing the degree of CCS recuperation that ensues from treatment with the therapeutic cell population.

### E. KITS

The present invention also provides kits. Any of the components disclosed herein may be combined in a kit. In certain embodiments the kits comprise one or more of a fast conducting cardiomyocyte population and/or slow conducting nodal cell population. The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the oligosphere subpopulations in a series are combined in a single container. In other embodiments, some or all of the oligosphere subpopulations in a series are provided in separate containers.

The kits of the present invention also will typically include packaging for containing the various containers in close confinement for commercial sale. Such packaging may include cardboard or injection or blow molded plastic packaging into which the desired containers are retained. A kit may also include instructions for employing the kit components. Instructions may include variations that can be implemented.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Generation of minKGFP BAC Transgenic Mice. A 200 kb BAC (RP23:276I20) containing the minK (Kcne1) locus was obtained from the Children's Hospital Oakland

Research Institute BACPAC Resource Center. PCR using Phusion DNA polymerase (NEB) and pEGFP-FRT-Zeo-FRT (gift of Boris Reizis and Philip Leider, Harvard Medical School) as template was used to generate a 1.6 kb fragment containing an EGFP-BGH pA-FRT-Zeo-FRT cassette flanked by 60 base pairs of homology to the minK genomic region. Italicized bases in the primers below represent regions of homology to the minK genomic region; bases in bold are homologous to the EGFP-pA-FRT-Zeo-FRT cassette; start and stop codons corresponding to the minK transcript are underlined minKGFPFwd:CTAAGTTGCCTTTTCCTTTCAGGAGTTTTGCTCTGCATCAGGGGAACCT TGACGCCCAGG A TGGTGAGCAAGGGCGAGGA (SEQ ID NO: 1); minKGFPRev:TATGGCAGGCATGCGACTAGAAAGATCCGCTTGTCACCTGTAG GGTGTGGGGTTCACGAC GGGAACAAAAGCTGGAGCTCG (SEQ ID NO: 2). Gel-purified PCR product was electroporated into DH10B cells harboring RP23R276:I20 that had previously been transfected with the RedET recombineering plasmid pSC101 (GeneBridges). BAC recombineering was then performed in E. coli according to the manufacturer's instructions. The FRT flanked zeocin resistance cassette was subsequently removed via arabinose induction of FLP recombinase in EL250 cells (Lee, et al., 2001). All constructs and BAC ends were sequence verified. minKGFP BAC DNA was purified using NucleoBond BAC-100 columns (Clontech). Circular BAC DNA was used for microinjections into fertilized CD-1 oocytes at The University of Chicago Transgenics Core Facility.

Generation of minKCreER^{T2} BAC Transgenic Mice. A 200 kb BAC (RP23:276I20) containing the minK (Kcnel) locus was purchased from the Children's

Hospital Oakland Research Institute BACPAC Resource Center. PCR using Phusion DNA polymerase (NEB) and pCreER^{T2} (gift of Boris Reizis and Philip Leider, Harvard Medical School) as template was used to generate a 2.8 kb fragment containing a CreER^{T2}-BGH pA-FRT-Zeo-FRT cassette flanked by 60 base pairs of homology to the minK genomic region. Italicized bases in the primers below represent regions of homology to the minK genomic region; bases in bold are homologous to the CreERT2-pA-FRT-Zeo-FRT cassette; start and stop codons corresponding to the minK transcript are underlined minKCreERTFwd:CTAAGTTGCCTTTTCCTTTCAGGAGTTTTGCTCTGCATCAGGGGAA CCTTGACGCCCAGG ATGTCCAATTTACTGACCGTACACC (SEQ ID NO: 3); minKCreERTRev: TATGGCAGGCATGCGACTAGAAAGATCCGCTTGTCACCTGTAGGGTGTGGGGTTCACG AC GGGAACAAAAGCTGGAGCTCG (SEQ ID NO: 4). Gel-purified PCR product was electroporated into DH10B cells harboring RP23R276:I20 that had previously been transfected with the RedET recombineering plasmid pSC101 (GeneBridges). BAC recombineering was then performed in E. coli according to the manufacturer's instructions. The FRT flanked zeocin resistance cassette was subsequently removed via arabinose induction of FLP recombinase in EL250 cells (Lee, et al., 2001). All constructs and BAC ends were sequence verified. minK:CreER^{T2} BAC DNA was purified using NucleoBond BAC-100 columns (Clontech), then digested with NotI to linearize the BAC and remove the pBACe3.6 vector. Linearized BAC containing the minKCreER^{T2} cassette was purified away from vector sequence and a 34 kb fragment resulting from an internal NotI site using a Sepharose 4B-CL column (Sigma). Microinjections of fertilized CD-1 oocytes were performed by the University of Chicago transgenics core facility.

Tamoxifen Administration. Tamoxifen free base (MP Biomedical) was dissolved in corn oil at a concentration of 20 mg/mL. Tamoxifen was administered to adult mice via oral gavage at a dose of 0.167 mg/g body weight for 5 consecutive days. Tamoxifen was administered via gavage to timed pregnant females at a fixed dose of 4 mg at E9.5

X-Gal Staining. For whole X-Gal staining on frozen sections, hearts were removed and dissected into ice-cold PBS and subsequently frozen in OCT in the gas phase of liquid nitrogen. 10 μM cryosections were mounted on slides, air-dried, and fixed for 10 min in 0.2% glutaraldehyde in PBS. Slides were then rinsed in PBS and X-Gal wash solution (0.1 M phosphate buffer pH 7.3, 2 mM MgCl₂) and stained in X-Gal stain solution (0.1 M phosphate buffer, 50 mM Tris, 2 mM MgCl₂, 5 mM potassium ferricyanide, 5 mM potassium ferrocyanide, 1 mg/mL X-Gal) overnight at 37° C. Slides were subsequently counterstained with Nuclear Fast Red, dehydrated, and mounted.

For whole mount staining of adult hearts, hearts were dissected into ice-cold PBS and the left and right ventricular free walls were opened to facilitate entry of staining solutions. Hearts were then fixed for 60 minutes in 4% paraformaldehyde in PBS at 4° C., then rinsed in PBS followed by X-Gal wash solution containing 0.01% deoxycholate and 0.02% NP-40. Hearts were stained overnight in X-Gal stain solution containing 0.01% deoxycholate and 0.02% NP-40 at 37° C., and washed and post-fixed in 10% neutral buffered formalin prior to photographing. Embryos were stained for X-Gal in the same manner as described above, except that fixation was performed for 45 minutes.

Acetylcholinesterase Activity. Hearts were dissected into ice-cold PBS and subsequently frozen in OCT in the gas phase of liquid nitrogen. 7 μM cryosections were mounted on slides, air-dried, and fixed in 4% paraformaldehyde in PBS for 10 minutes at room temperature. Slides were then stained for acetylcholinesterase activity overnight at 37° C. in acetylcholinesterase staining solution (1.7 mM acetylthiocholine iodide, 38 mM sodium acetate, 63.2 mM acetic acid, 4.8 mM sodium citrate, 3 mM cupric sulfate, 0.5 mM potassium ferricyanide, 0.08 mM iso-OMPA) as described (El-Badawi, et al., 1967).

Immunofluoresence. Hearts were removed and dissected into ice-cold PBS and subsequently frozen in OCT in the gas phase of liquid nitrogen. 7 μM cryosections were mounted on slides, air-dried, and fixed for 10 min in 4% paraformaldehyde. Sections were permeabilized in 1% Triton-X100 in PBS for 10 minutes, then blocked in 10% normal goat serum in PBS-T for 30 minutes at room temperature. Immunofluoresence experiments using a primary antibody raised in mouse included an additional mouse IgG blocking step in 0.13 mg/mL F(ab) fragment goat anti-mouse IgG (Jackson ImmunoResearch) in PBS for 60 minutes at room temperature. Sections were incubated overnight in primary antibody diluted in blocking buffer at 4° C., rinsed in PBS, then incubated in secondary antibody diluted in blocking buffer for 60 minutes at room temperature. Slides were mounted in VectaShield+DAPI (Vector Laboratories) or counterstained with DAPI and mounted in ProLong Gold (Invitrogen) prior to visualizing fluorescence. Primary antibodies were as follows: rabbit polyclonal anti connexin 40 (Zymed 36-4900, 1:250), mouse monoclonal anti connexin 43 (BD Transduction 610061, 1:250), rabbit polyclonal anti HCN-4 (Millipore 5808, 1:250), rabbit polyclonal anti Naᵥ1.5 (Alomone Labs, ASC-500 1:250), goat polyclonal anti Tbx5 (Santa Cruz sc-17866, 1:250), and goat polyclonal anti contactin-2 (R&D AF1714, 1:50). Secondary antibodies used were goat anti-rabbit IgG AlexaFluor-488 and goat anti-mouse IgG F(ab)₂ AlexaFluor-594 (Molecular Probes) for experiments with co-staining for rabbit and mouse primary antibodies, and donkey anti-rabbit IgG AlexaFluor-488 (Molecular Probes), and donkey anti-goat IgG AlexaFluor-594 (Molecular Probes) for co-staining with rabbit and goat primary antibodies.

In situ hybridization. Non-radioactive slide whole mount in situ hybridization was performed on paraffin sections with DIG-labeled probe as previously described (Moskowitz, et al., 2007; Moorman, et al., 2001), with the addition of an RNAse A treatment (30 min in 20 ug/mL RNAse A at 37° C.) following hybridization. Whole mount in situ hybridization was performed on E10.5 CD-1 wild type embryos as described (Hoffmann, et al., 2009). DIG-labeled probes were prepared using previously described plasmids: Tbx3, Cx43 (generous gifts of the Christoffels lab, University of Amsterdam Academic Medical Center), Id2 (Moskowitz, et al., 2007), were obtained from the IMAGE consortium (Bmp2), or synthesized from T7-tailed PCR products using E14.5 heart cDNA as template.

FACS Sorting and RNA Extraction. minKGFP BAC transgenic embryos were obtained from timed pregnancies and rapidly dissected into ice-cold PBS. Hearts were subsequently isolated, minced, and digested in enzyme buffer (0.25 mg/mL Collagenase I (Worthington), 0.4 mg/mL pancreatin (Gibco), 0.15 ug/mL DNAse I (Roche) in Ca⁺⁺ and Mg⁺⁺ free HBSS supplemented with 10 mM HEPES) at 37° C. Hearts were incubated for 10 min at 37° C. with shaking, then allowed to settle for 5 min; the supernatant containing single cells was removed, centrifuged, resuspended in PBS+2% FBS, and placed on ice. This process was repeated 3-4 times until all tissue was digested. Fractions were combined, washed with PBS, and passed through a 40 uM filter. Propidium iodide was added immediately before FACS to allow for live/dead discrimination. Cells were sorted on a Beckman-Coulter MoFlo instrument housed at the University of Chicago Flow Cytometery Core directly into Trizol LS (Invitrogen). RNA was extracted according to the manufacturer's instructions.

Semi-quantitative RT-PCR. RNA was treated with RNAse free DNAse I (Invitrogen) then reverse transcribed to cDNA with SuperScript III (Invitrogen) and anchored oligo dT₂₀ primers (IDT). Semi-quantitative RT-PCR was performed with equal amounts of cDNA using JumpStart Red Taq (Sigma). The linear range for each primer pair was determined by removing samples in 2 cycle increments, and results shown are representative of at least 3 biological replicates.

Microarray Analysis. For microarray analysis, RNA was extracted from 4 groups of 9-14 E10.5 minKGFP transgenic hearts as described above. RNA integrity was analyzed using an Agilent BioAnalyzer and RIN values were above 8 for all samples. 5 ng of total RNA was amplified using the NuGen WT Ovation Pico kit. 2 ug of amplified product was subsequently labeled with AlexaFluor-3 using the BioPrime Total Labeling Kit (Invitrogen) according to the manufacturer's instructions. 3 ug of labeled product was hybridized to Agilent Mouse Whole Genome expression 4×44 k arrays (G4122F). Array hybridizations were performed at The University of Chicago, Argonne National Labs high throughput genome analysis core facility, according to the manufacturers' instructions. Microarray analysis was performed in collaboration with Tom Stricker at The University of Chicago. Agilent Feature Extraction software was used to extract feature intensities and to flag saturated, non-uniform, and outlier features, which were removed from subsequent analysis. Probe intensity was adjusted by subtracting background intensity using the minimum method (Gentleman, 2005; Ritchie, et al., 2007) and quantile normalized between arrays (Bolstad, et al., 2003). Probes that were undectable across all 8 arrays and low variance probes (interquartile range cutoff of 0.5) were filtered out. The remaining probes were analyzed for differential expression in limma (Smyth, et al., 2004), using a design matrix with terms for mouse pair and GFP status. P values were adjusted for multiple hypothesis testing in limma using Benjamini and Hochberg's method to control the false discovery rate. Microarray data conforms to MIAME guidelines and has been deposited in the NCBI Gene Expression Omnibus under accession #GSE36136.

Optical Mapping. AV conduction delay and ventricular activation patterns were assessed with high resolution optical mapping in collaboration with Carolina Vasquez and Gregory Morley at the NYU School of Medicine. Mapping was performed on an upright microscope (Olympus BX51WI) equipped with a reflected light fluorescence attachment, a 100 W mercury arc lamp and a CMOS camera (MiCAM ULTIMA-L, SciMedia) as previously described (Leaf, et al., 2008). Recordings were made at 1,000 frames per second with a 100×100 pixel array, providing a spatial resolution of 25.0 um. Hearts were removed from E12.5 embryos and dissected into ice-cold PBS. Prior to mapping, hearts were loaded with the voltage sensitive dye di-4-ANEPPS (20 uM; Invitrogen) for 6 minutes at 37° C. and maintained in recording solution (1% FBS in Hank's balanced salt solution at 37 ° C., pH 7.4) throughout the mapping procedure. Movies were obtained during sinus rhythm in the absence of motion reduction techniques. Individual beats were averaged over the course of a 4 s recording interval using an autocorrelation algorithm to improve the signal-to-noise ratio and analyzed as previously described (Leaf, et al., 2008; Morley, et al., 1999). Analysis was performed blinded to genotype.

Telemetry ECG Analysis. 10-12 week old mice were anesthetized with isofluorane and telemetry transmitters (DSI ETA-F10) were implanted in the back with leads tunneled to the right upper and left lower thorax, as previously described (Wheeler, et al., 2004). Heart rate, PR, and QRS intervals were calculated using Ponemah Physiology Platform (DSI) from 24 hour recordings.

Electrophysiology Studies. Electrophysiology studies were performed in collaboration with Fang Liu, Kurt Schillinger, and Vic Patel at The University of Pennsylvania. Detailed protocols for in vivo electrophysiology studies have been previously described (Liu, et al., 2008). 10-12 week old mice were anesthetized with pentobarbital (33 mg/kg ip) and a 1.1-Fr octapolar electrode catheter (Millar; EPR-800) was advanced via a right jugular venous cut-down to record right atrial, His bundle, right ventricular potentials and perform programmed electrical stimulation. For studies involving atropine, surface ECG and intracardiac recordings were obtained, then repeated after mice were injected with 20 mg/kg atropine.

Echocardiography Studies. Echocardiography studies were performed in collaboration with Gene Kim at the University of Chicago. Transthoracic echocardiography in mice was performed under inhaled isoflurane for anesthesia, delivered via nose cone. Chest hairs were removed with a topical depilatory agent. Limb leads were attached for electrocardiogram gating, and animals were imaged in the left lateral decubitus position with a VisualSonics Vevo 770 machine using a 30 MHz high-frequency transducer. Body temperature was maintained using a heated imaging platform and warming lamps. Two-dimensional images were recorded in parasternal long- and short axis projections, with guided M-mode recordings at the midventricular level in both views. LV cavity size and percent fractional shortening were measured in at least 3 beats from each projection and averaged. LV fractional shortening ([LVIDd−LVIDs]/LVIDd) and LV chamber dimensions were calculated from the M-mode measurements.

Cell Culture Studies. The Scn5a 3′ enhancer (chr9:119378051-119379479; NCBI build 37/mm9) was cloned from BAC RP23-103G4 (Invitrogen) into the XhoI and BglII sites of a pGL3 Basic vector (Promega) that was modified to include a minimal TK promoter between the BglII and HindIII sites. Conserved Tbx5 binding elements were identified using the ECR Browser (Ovcharenko, et al., 2004) and rVISTA (Ovcharenko, et al., 2004) software programs and have the following genomic coordinates: TBEl: chr9:119,379,020-119,379,031; TBE2: chr9:119,378,997-119,379,008; TBE3: chr9:119,378,918-119,378,929. Site directed mutagenesis was performed using the QuikChange Lightning kit (Agilent). Tbx5 was cloned from a mouse atrial cDNA library into pcDNA3.1 hygro (Invitrogen). All constructs were sequence verified.

Transient transfections were performed in HEK-293T cells using FuGene HD (Promega) according to the manufacturer's instructions. The day before transfection, 2×10⁵ cells/well were plated in 12 well plates in growth medium (D-MEM+10% FBS and L-glutamine). The following day, each well was transfected with 600 ng of luciferase reporter, 400 ng of Tbx5 or empty pcDNA vector, and 1 ng of pRL-CMV using a 3:1 FuGene HD:DNA ratio. Growth medium was replaced 24 hours after transfection, and cells were harvested and luciferase activity was assayed 48 hours after transfection using the Promega Dual Luciferase Reporter kit according to the manufacturer's instructions. All transfections were performed in duplicate and repeated in a minimum of 3 independent replicates.

Transient Transgenic Experiments. The Scn5a enhancer and TBE mutant enhancer were subcloned from the pGL3 reporter vector into the XhoI and PstI sites of the Hsp68-LacZ transgenic reporter vector (Kothary, et al., 1989). The enhancer-Hsp68-LacZ fragment was digested with XhoI and NotI, gel purified, and used to create transient transgenic embryos at the University of Chicago Transgenic Core Facility. Embryos were harvested at E13.5, stained with X-Gal overnight at 37° C. as previously described (Hoffmann, et al., 2009), embedded in paraffin, sectioned at 5 uM and counterstained with Nuclear Fast Red.

Western Blotting. Atria and ventricles were dissected from Tbx5^{fl/fl} mice and Tbx5^{minKCreERT2} littermates administered tamoxifen at 6-7 weeks of age and studied 4-5 weeks following tamoxifen administration. Tissue was snap frozen in liquid nitrogen, pulverized, and homogenized in RIPA buffer (50 mM Tris-HCL pH 8, 150 mM NaCl, 1% Triton-X, 0.50% sodium deoxycholate, 0.1% SDS, 5 mM EDTA+1 Roche EDTA-Free complete protease inhibitor tablet/50 mL buffer). Samples were tumbled for 1 hour at 4° C., then centrifuged for 10 minutes at 13,200×g. Protein concentration was determined using the BCA assay (Pierce) with BSA as a standard. 25 ug of protein was diluted in Laemmli buffer, heated for 10 min at 70° C. and subjected to SDS-PAGE on 4-20% TGX gels (Bio-Rad). Proteins were transferred to nitrocellulose membranes, blocked with 5% milk in TBS-T, and incubated with primary antibody overnight at 4° C. Primary antibodies were: rabbit anti-Naᵥ1.5 (Alomone ASC-005, 1:2000), rabbit anti-Cx40 (Zymed 36-4900, 1:500), and rabbit anti Hsp90 (Santa Cruz Biotechnology sc-7947, 1:3000). After rinsing in TBS-T, membranes were incubated in secondary antibody (goat anti-rabbit-HRP, Jackson Immuno-Research, diluted 1:3000 in 2.5% milk), rinsed, and visualized using ECL Prime (Amersham).

### Example 2

### A Role for BMP Signaling in Molecular Specification of the AV Bundle

A barrier to identifying the transcriptional networks required for conduction system development has been an inability to isolate conduction system cells early in development.

To address this problem, a bacterial artificial chromosome (BAC) transgenic mouse line, minKGFP, that expresses high levels of EGFP throughout the developing conduction system was created. This transgenic mouse line was then used to transcriptionally profile the developing CCS at E10.5, the first time point at which apex to base ventricular depolarization is present (Rentschler, et al., 2001). Analysis of this transcriptional profile identified novel markers of the E10.5 primary ring and suggested a potential role for BMP signaling genes in development of the AV bundle. This hypothesis was tested by assessing AV bundle specification in the absence of Smad4, a co-SMAD required for canonical BMP signaling (Derynck, et al., 2003). In the absence of Smad4, Tbx3 and Id2, key regulators of VCS development (Moskowitz, et al., 2007) were downregulated, indicating a novel role for canonical BMP signaling in the molecular specification of the AV bundle. Loss of Smad4 did not result in complete misspecification of the AV bundle, however, as Cx43 was still excluded from the developing AV bundle and ventricular activation was not affected by loss of Smad4.

minKGFP Marks the Developing Conduction System. The minK locus was chosen to drive GFP expression within the conduction system based on previous work demonstrating that replacement of the minK coding exon with lacZ resulted in CCS-specific β-galactosidase expression in the heart from E9 through adulthood (Moskowitz, et al., 2007; Kupershmidt, et al., 1999; Kondo, et al., 2003). The single minK coding exon was replaced with EGFP in RP23-276120, a BAC containing 199 kb of mouse genomic DNA including the minK locus, 144 kB of DNA upstream of the minK transcriptional start site and 45 kb of DNA downstream of the last exon (A), by homologous recombination in E. coli (BAC recombineering ([Copeland, et al., 2001]). The recombined minKGFP BAC was then used to generate a stable line of minKGFP BAC transgenic mice.

EGFP expression in minKGFP BAC transgenic mice was compared to β-galactosidase activity in minK^{Lacz} mice. At E10.5, GFP expression demarcated rings of tissue surrounding the atrioventricular canal, interventricular ring, and outflow tract (B), specialized structures included in descriptions of the cardiac conduction system based on morphological, immunohistochemical, and functional criteria (Viragh, et al., 1977a; Viragh, et al., 1977b; Wessels, et al., 1992; Wenink, et al., 1976; Ikeda, et al., 1990; Anderson, et al., 1974; Anderson, et al., 2009; Lamers, et al., 1987). These regions coincide with those described as primary myocardium (Moorman, et al., 2003). minKGFP expression marked the developing central conduction system at E 14.5 (C) and the mature central conduction system of adult mice, as assessed by a comparison of EGFP fluorescence with B-galactosidase activity in minK^{LacZ} mice (D).

Having established that GFP expression in minKGFP BAC transgenic mice marks the conduction system, FACS was performed to isolate cells of the developing CCS. E10.5 or E14.5 hearts were digested to single cells, then sorted on the basis of GFP fluorescence intensity. A gating strategy was employed based on relative GFP fluorescence to collect three fractions of cells (A): (1) GFP negative cells demonstrating only background fluorescence (GFP^{ncg}); (2) GFP positive cells with low levels of GFP expression (GFP^{low}), defined by a chamber myocardial control composed of non-conduction atrial and left ventricular free wall myocardium and (3) GFP positive cells with a higher fluorescence intensity than the chamber myocardial controls (GFP^{high}).

RT-PCR on RNA extracted from each fraction supported the biological relevance of the gating scheme (B). Each fraction expressed equal amounts of Rpl4, a housekeeping gene (de Jonge, et al., 2007). The expression of minK and GFP was consistent with the gating protocol: the GFP^{neg} fraction of cells did not express minK or GFP, while GFP^{low} and GFP^{high} cells expressed low and high levels, respectively, of minK and GFP. GFP^{neg} cells did not express myosin heavy chain (Mhc), a myocyte marker, but did express high levels of Pecam-1 and Pdgfr-b, markers of endothelial and vascular support cells, respectively (Hellstrom, et al., 1999; Sugi, et al., 2004), suggesting that GFP^{neg} cells represented the non-cardiomyocytes within the developing heart. GFP^{low} and GFP^{high} cells both expressed high levels of Mhc, but low levels of Pecam-1 and Pdgfr-b, indicating that both of these fractions were primarily composed of cardiomyocytes. While low levels of Pecam-1 and Pdgfr-b expression were observed in the GFP^{low} fraction, particularly at E14.5, this is believed to be due to low levels of contamination between fractions in the sorting procedure.

GFP^{high} cells expressed high levels of the extensively validated conduction system marker Tbx3 and excluded Anf (Nppa) and Cx43, chamber markers known to be excluded from the CCS (Christoffels, et al., 2009; Hoogaars, et al., 2004). From these experiments it was concluded that while GFP is expressed throughout the embryonic myocardium in minKGFP BAC transgenic mice, it is expressed at the highest levels in the developing conduction system: the molecular profile of isolated cells is consistent with that described for the domains that exhibit high levels of EGFP fluorescence. While embryonic β-galactosidase activity in minK^{LacZ} knockin mice has been reported to be conduction system specific (Kupershmidt, et al., 1999; Kondo, et al., 2003), these conclusions are consistent with the reported endogenous expression of the minK transcript (Honore, et al., 1991; Felipe, et al., 1994; Franco, et al., 2001) as well as experience with embryonic tamoxifen inducible Cre activity driven from the minK locus in the BAC used in this study (Arnolds, et al., 2011). From a functional standpoint, the key conclusion is that a population enriched for CCS cells can be isolated by FACS sorting embryonic minkGFP hearts.

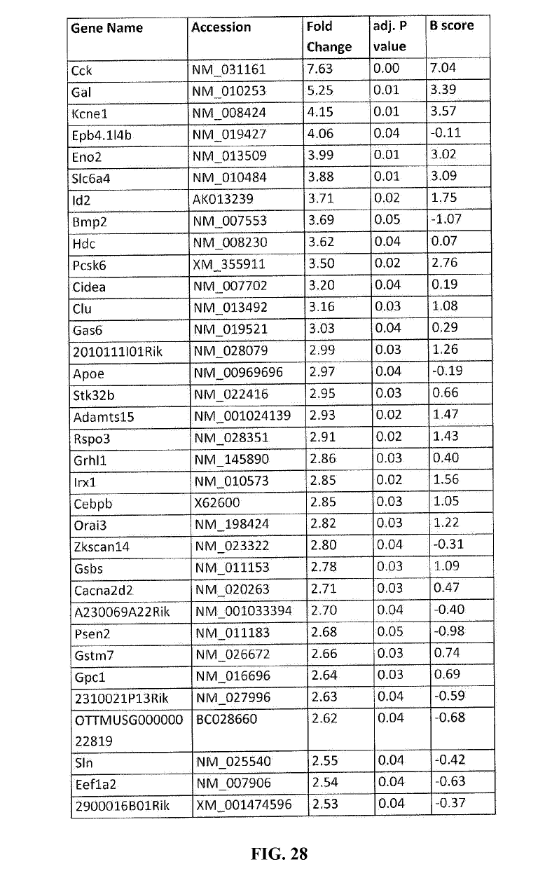

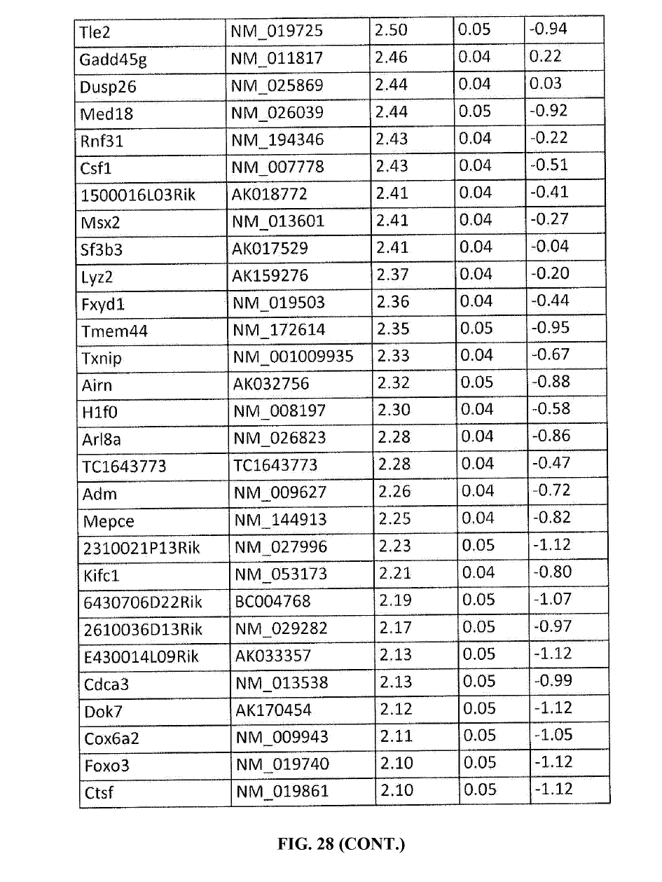

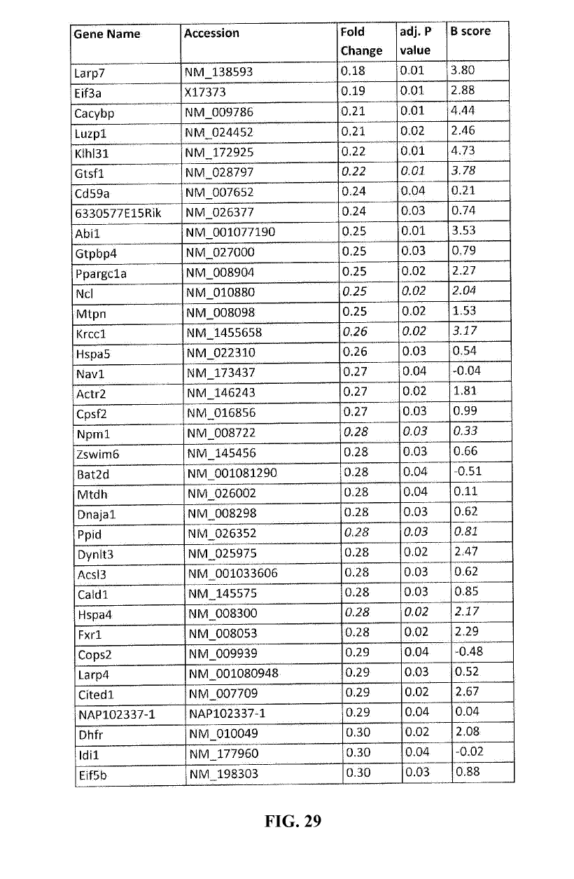

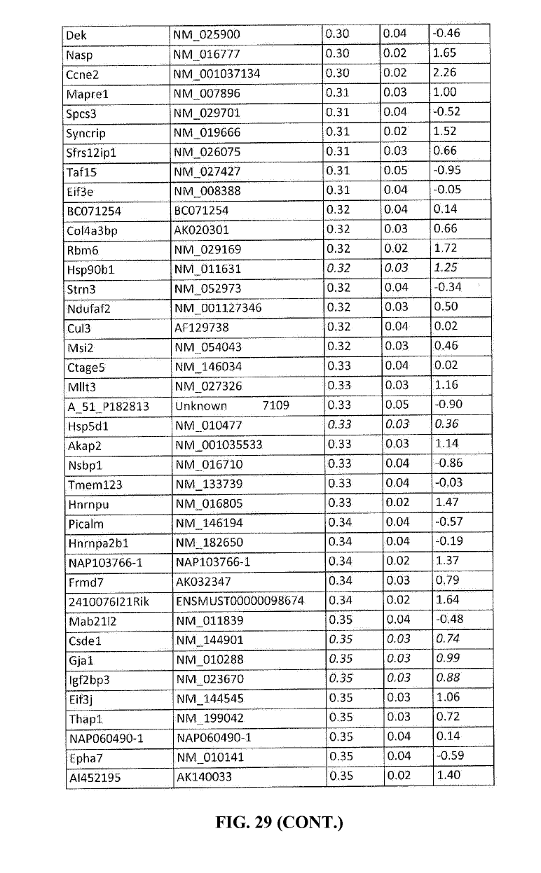

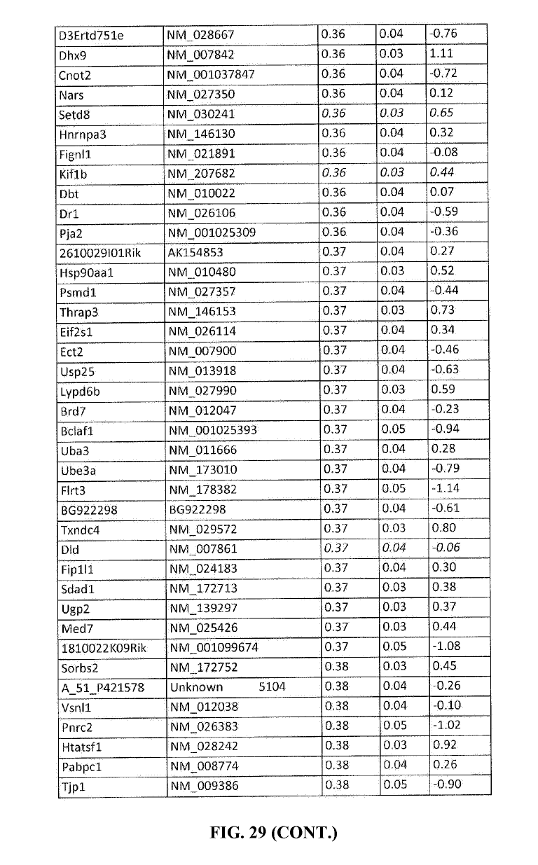

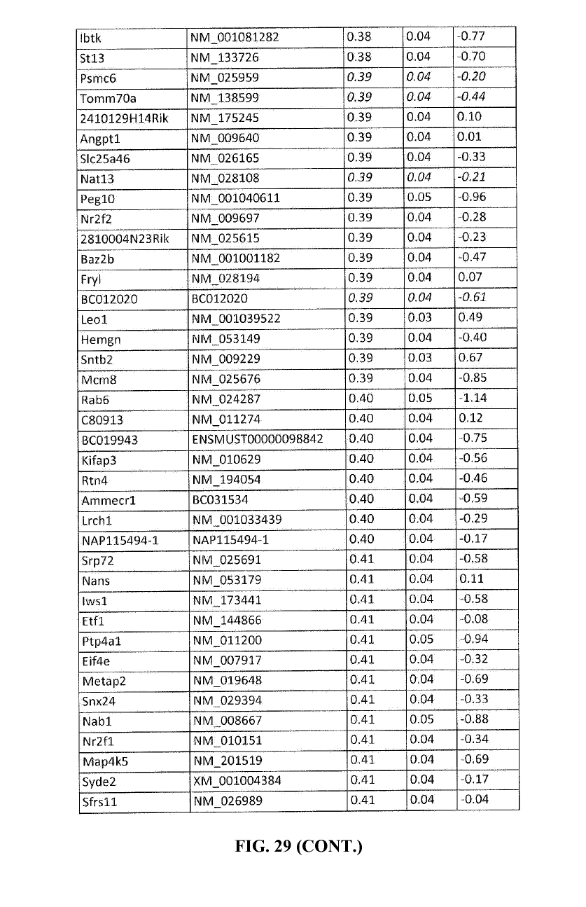

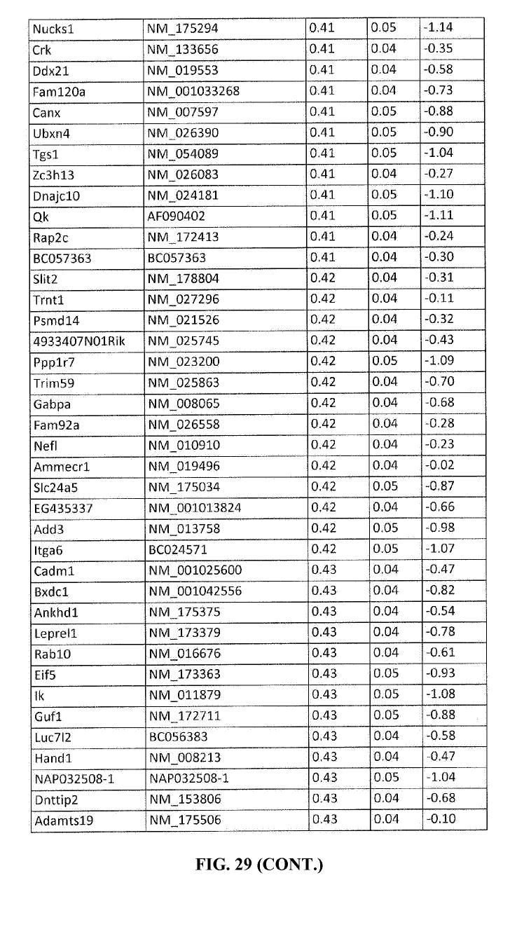

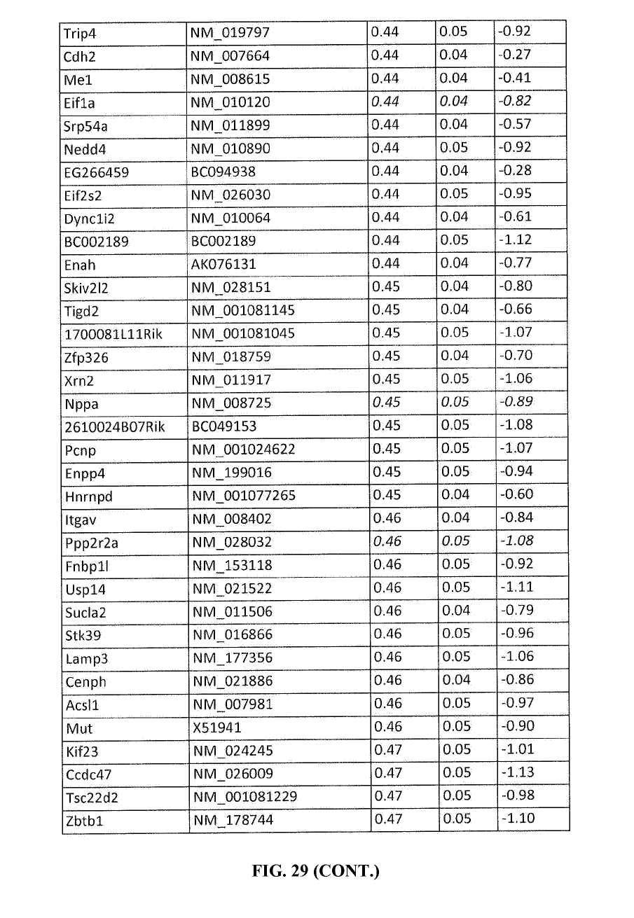

Transcriptional Profiling of the E10.5 Conduction System Indicates a Role for BMP Signaling in CCS Development. To identify pathways that may function in the specification of the cardiac conduction system, the transcriptional profile of minKGFP^{high} cells was compared with minkGFP^{low} chamber myocardium at E1 0.5, the earliest timepoint at which the VCS is functional (Rentschler, et al., 2001). RNA was extracted from 4 pools of E10.5 minKGFP hearts, amplified, and hybridized to Agilent mouse whole genome microarrays (C). Comparison of the transcriptional profiles of E10.5 minKGFP^{high} cells and minKGFP^{low} chamber myocardium identified 63 genes significantly upregulated in minKGFP^{high} cells and 241 genes significantly upregulated in minKGFP^{low} chamber cells to a p-value of less than 0.05 after correcting for multiple testing (C). Notably, 9 of the 63 upregulated genes have published expression patterns in the minKGFP^{high} domain; conversely the key chamber markers Anf, Cx43, and Citedl were all upregulated in minKGFP^{low} chamber cells (Table 2). These results supported the validity of the employed approach to identify genes specifically expressed in, or excluded from, the embryonic CCS. The full list of genes significantly upregulated in E10.5 minKGFP^{high} cells and E10.5 minKGFP^{low} cells are included in FIGS. 28 and 29, respectively.

TABLE 2
Microarray Identified Known Markers of the Conduction System.

| | Enrichment in | | |
|---|---|---|---|
| Gene | minKGFP^{high} CCS | Adj. p value | Reference |
| minK | 4.15 | 0.01 | (37, 38) |
| (KCNE1) | | | |
| Id2 | 3.71 | 0.02 | (18) |
| Irx1 | 2.85 | 0.02 | (76) |
| Msx2 | 2.41 | 0.04 | (77, 78) |
| Bmp2 | 3.69 | 0.05 | (77, 78) |
| Rspo3 | 2.91 | 0.02 | (79) |
| Grhl1 | 2.86 | 0.03 | (80) |
| Ga1 | 5.25 | 0.01 | (81) |
| LysM | 2.37 | 0.04 | (82) |
| Cited1 | 0.28 | 0.04 | (83) |
| Cx43 | 0.35 | 0.03 | (17) |
| (Gja1) | | | |
| Anf | 0.45 | 0.05 | (16) |
| (Nppa) | | | |

Genes known to be expressed in, or excluded from, the developing conduction system were identified in the microarray analysis of E10.5 conduction system.

TABLE 3
BMP Pathway Genes Unregulated in minKGFP^{high} CCS Cells.

| | Enrichment in | |
|---|---|---|
| Gene | minKGFP^{high} | Adj. p value |
| Bmp-2* | 3.69 | 0.05 |
| Id2* | 3.71 | 0.02 |
| Grhl1 (84) | 2.86 | 0.03 |
| Msx2* | 2.41 | 0.04 |
| Csf1* | 2.43 | 0.04 |
| ApoE (85) | 2.97 | 0.04 |
| Irx1* | 2.85 | 0.02 |
| Pcsk6 (86) | 3.5 | 0.02 |
| Gpc1 (87) | 2.64 | 0.03 |
| Gas6* | 3.03 | 0.04 |
| Txnip* | 2.33 | 0.04 |
| Clu* | 3.16 | 0.03 |
| Rnf31* | 2.43 | 0.04 |
| Adm* | 2.26 | 0.04 |
| Foxo3* | 2.10 | 0.05 |
| S1c6a4* | 3.88 | 0.01 |
| Cck* | 7.63 | 0.002 |
| Gsbs* | 2.78 | 0.03 |
| Cox6a2* | 2.11 | 0.05 |
| Psen2* | 2.68 | 0.05 |
| Gal* | 5.25 | 0.01 |

BMP pathway genes upregulated in minKGFP^{high} CCS cells at E10.5 were identified via Ingenuity Pathway Analysis (indicated with *) or via manual curation of the literature (reference indicated in parentheses).

Further interrogation of the microarray data revealed overrepresentation of genes related to BMP signaling, assessed by Ingenuity Pathway Analysis and manual curation of genes upregulated in minKGFP^{high} cells (Table 3). GFP is expressed at high levels in E10.5 minKGFP BAC transgenic hearts in the outflow tract, AV canal, and primary (interventricular) ring, considered the precursor to the AV bundle (Miquerol, et al., 2011). BMP signaling has described roles in development of the outflow tract and AV canal (Wang, et al., 2011), but has not been implicated in the development of the AV bundle or bundle branches, central components of the VCS.

To determine whether identification of BMP signaling genes in the minKGFP^{high} fraction solely reflected the inclusion of the outflow tract and AV canal amongst the profiled cells or reflected a potentially unappreciated role for BMP signaling in development of the AV bundle, the E10.5 expression pattern of BMP-related genes upregulated in minKGFP^{high} cells was examined. A putative role for BMP signaling in the developing VCS requires the presence of a BMP ligand. Bmp2 was upregulated in the microarray and has been previously reported to be expressed in the AV cushions, as well as the developing interventricular septum in the chick and mouse (Abdelwahid, et al., 2011; Somi, et al., 2004; Yamada, et al., 2000). The inventors verified expression of Bmp2 in the developing AV canal and primary ring at E10.5 (A). Id2 is a BMP target, plays a key role in specification of the VCS (Moskowitz, et al., 2007; Hollnagel, et al., 1999; Song, et al., 2007; Moskowitz, et al., 2011), and was upregulated in minKGFP^{high} CCS cells, but its expression pattern has not been examined in the E10.5 primary ring. Robust Id2 expression was found in tissues adjacent to those expressing Bmp2 (B); specifically Id2 was strongly expressed in the developing interventricular septum, in addition to the AV canal and the mesenchyme of the AV cushions. The Irx family of transcription factors plays key roles in patterning electrical activity in the heart (Costantini, et al., 2005; Zhang, et al., 2011). Irx1 was upregulated in minKGFP^{high} CCS cells, consistent with previous work demonstrating specific expression in the developing VCS in both mice and zebrafish (Christoffels, et al., 2000; Joseph, et al., 2004). Msx2 was significantly upregulated in minKGFP^{high} CCS cells and is a direct BMP-2 target (Delot, et al., 2003) with known expression in the developing conduction system (Abdelwahid, et al., 2001; Somi, et al., 2004). While Msx2 knockout mice do not have conduction defects (Jay, et al., 2005), Msx2 is known to act in concert with Msxl and T-box factors to suppress Cx43 (Boogerd, et al., 2008). The transcription factor Tbx3, which is required for specification of the AV bundle (Bakker, et a1.,2008), is a BMP target in the AV canal (Singh, et a;., 2012; Yang, et al.). Together these findings demonstrated the ability of the cell sorting protocol to identify genes with CCS expression and suggested a potential role for BMP signaling in the developing VCS.

Ingenuity Pathway Analysis performed on the microarray data also identified a large cohort of BMP pathway genes without a previously reported VCS expression pattern. The inventors evaluated the expression of these genes at E10.5 and identified multiple novel markers of the primary ring (C-F). Slc6a4 encodes a serotonin transporter and showed specific expression in the E10.5 primary (interventricular) ring (C). Serotonin signaling is known to play a role in cardiac conduction (Monassier, et al., 2010). Gsbs, encoding a G-protein substrate with a role in motor learning (Endo, et al., 2009), Gpc1, encoding a heparan sulfate proteoglycan with a potential role in modulating extracellular signaling (Filmus, et al., 2008), and Adm, encoding the peptide hormone adrenomedullin, all showed strong expression in the E10.5 primary ring, considered the precursor to the AV bundle (D-F). Csf1, Clu, Psen2, ApoE, Rnf31, Pcsk6, Gas6, Foxo3, Txnip, and Cox6a2 were expressed in, but not specific to, the developing CCS (data not shown). Taken together these findings demonstrate the validity of the utilized cell sorting/microarray approach for identifying genes with differential expression in the minKGFP^{hih} domain.

Overall, upregulation of a BMP ligand, Bmp2, BMP targets with known roles in conduction system development, and multiple genes with established or putative roles in the BMP signaling pathway in minKGFP^{high} cells suggested a role for BMP signaling in the developing VCS, specifically cells of the primary ring which gives rise to the AV bundle.

Removal of SMAD4 from the mef2c-AHF-Cre Domain Affects Molecular Specification of the AV Bundle. The BMP signaling pathway has tremendous redundancy at the ligand and receptor level, with overlapping expression of BMP ligands and receptors in the embryonic heart (Euler-Taimor, et al., 2006; van Wijk, et al., 2007). The inventors therefore chose to target Smad4, a co-SMAD required for transduction of canonical BMP signaling, to test the hypothesis that canonical BMP signaling is required for specification of the AV bundle. To circumvent the early embryonic lethality of Smad4 null embryos, (Sirard, et al., 1998; Yang, et al., 1998), the mid-gestational lethality following cardiomyocyte-specific removal of Smad4 (Song, et al., 2007; Qi, et al., 2007; Azhar, et al., 2010), and selectively target Smad4 in the VCS portion of the CCS, the inventors conditionally removed Smad4 from the mef2c-AHF-Cre lineage. The mef2c-AHF-Cre lineage contributes to the AV bundle, as well as the ventricular septum, right ventricle, and outflow tract, but is excluded from the AV ring and AV node (Verzi, et al., 2005; Aanhaanen, et al., 2010).

The inventors evaluated the role of Smad4 in specification of the AV bundle by performing in situ hybridization for CCS markers in Smad4^{mef2c-AHF-Cre/-} mutant embryos at E14.5. Id2 and Tbx3 play key functional roles in specifying the VCS, while Cx43 is repressed by Tbx3 and excluded from the AV bundle (Moskowitz, et al., 2007; Bakker, et al., 2008). Decreases in Id2 and Tbx3 expression were observed in the AV bundle of Smad4^{mef2c-AHF-Cre/-} hearts compared to wild type littermate control embryos, demonstrating a role for canonical BMP signaling in the specification of the AV bundle. This is consistent with previous findings that removal of Smad4 with Nkx2.5-Cre resulted in modest reductions in cardiac Id2 expression at E11.5 (Song, et al., 2007). Cx43 was excluded from the AV bundle of Smad4^{mef2c-AHF-Cre/-} as well as Smad4^{fl/+} littermate control embryos. These observations suggest impaired molecular specification of the AV bundle in the absence of Smad4, but also demonstrate that removal of Smad4 from the mef2-AHF-Cre domain does not result in wholesale conversion of the VCS into working myocardium.

Removal of Smad4 from the mef2c-AHF-Cre Domain Does Not Affect VCS Function at E12.5. To determine if removal of Smad4 from the mef2c-AHF-Cre domain affects function of the VCS, the inventors performed optical mapping studies on E12.5 Smad4^{mef2c-AHF-Cre/-} hearts and Smad4^{fl/+} littermate controls. There were no differences in the A-V interval or ventricular activation pattern between Smad4^{mef2c-AHF-Cre/-} and Smad4^{fl/-} littermate controls.

In summary, the minKGFP BAC transgenic mice provide a tool to drive high levels of

EGFP expression in the conduction system of the mouse heart. Transcriptional profiling of cells isolated at E10.5 that express high levels of GFP indicated upregulation of BMP signaling in the minKGFP^{high} domain. This gene set includes genes with strong expression in the primary ring, considered the precursor to the AV bundle, indicating that BMP signaling plays a previously unexplored role in specification of the proximal ventricular conduction system. Removal of Smad4 from the mef2-AHF-Cre lineage, which contributes to the AV bundle, resulted in reductions in Tbx3 and Id2 expression, thereby implicating BMP signaling in the molecular specification of the AV bundle. Removal of Smad4 did not result in wholesale loss of AV bundle specification, however, and ventricular activation patterns were not affected.

### Example 3

### Inducible Recombination in the Cardiac Conduction System of MINKCREER^{T2} BAC Transgenic Mice

Many genes with key roles in the conduction system also play important roles in nonconduction (working) myocardium. Thus, the inability to perform tissue specific knockouts in the CCS has posed a major obstacle impeding progress towards investigating the potential CCS role of the wealth of targets identified by GWAS and other methods.

Inducible Cre activity allows for spatial and temporal control over recombinase activity, allowing for precisely targeted modulation of gene activity (Nagy, et al., 2000).

Such strategies have successfully targeted several cell types that contribute to the heart, yet it has not currently been applied to the specialized conduction system. The two inducible Cre drivers currently available with some specificity for the conduction system, HCN4:CreERT2 (Hoesl, et al., 2008) and Cx40:CreERT2-IRESmRFP (Beyer, et al., 2010) have significant potential limitations. Tamoxifen-inducible Cre activity in HCN4:CreERT2 mice has been reported in the SA node and AV node, but not the AV bundle or bundle branches (Hoesl, et al., 2008). Within the CCS, tamoxifen-inducible Cre activity in Cx40:CreERT2 mice has been reported in the AV bundle, bundle branches, and Purkinje fibers, but not the AV node (Beyer, et al., 2010). Tamoxifen-inducible Cre activity in Cx40:CreERT2 mice is not conduction system specific, with Cre activity also present in atrial cardiomyocytes and coronary vessels. Furthermore, both the HCN4:CreERT2 and Cx40:CreERT2 mice were produced by targeted recombination (knock-in) approaches, whereby an endogenous allele is replaced by the CreERT2 construct. Given that HCN4 and Cx40 are both ion channels with important functional roles in the conduction system (Bevilacqua, et al., 2000; Baruscotti, et al., 2011; Hagendorff, et al., 1999; Herrmann, et al., 2007; Kirchoff, et al., 1998; Simon, et al., 1998), haploinsufficiency for HCN4 or Cx40 complicates the interpretation of loss of function studies performed with these models.

The inventors developed an inducible CCS Cre transgenic mouse line targeting the minK locus. Previous studies demonstrated that β-galactosidase expression from the minKLacZ knockin allele is restricted to the AV node, AV bundle, and bundle branches in the adult mouse heart (Kupershmidt, et al., 1999; Moskowitz, et al., 2004; Viswanathan, et al., 2007). To circumvent concerns of haploinsufficiency resulting from knockin approaches, the inventors chose a BAC (bacterial artificial chromosome) recombineering (Copeland, et al., 2001) approach and targeted the minK (KCNE1) locus to generate mice expressing tamoxifen-inducible Cre in the CCS. The coding sequence of the minK locus was replaced with a CreERT2 cassette in BAC RP23276-I20 in E. coli. The modified BAC, which contains 107 kB upstream and 48 kB downstream of the minK locus, was then used for pronuclear injection into fertilized oocytes. Four independent transgenic lines were obtained with this approach, each demonstrating consistent tamoxifen-inducible Cre activity in the cardiac conduction system in preliminary experiments. A single line was selected for more detailed study.

To evaluate tamoxifen-inducible Cre activity in minK:CreERT2 BAC transgenic mice, minK:CreERT2 mice were crossed to ROSA26R reporter mice which constitutively express LacZ following Cre-mediated deletion of a loxP flanked STOP codon (Soriano, et al., 1999). Tamoxifen was administered to 6-7 week old mice by oral gavage for 5 consecutive days, and 13-galactosidase activity was assayed in whole mount and on histologic sections. Following tamoxifen administration, X-Gal staining demonstrated consistent recombination at the crest of the interventricular septum and in stereotyped patterns on the left and right septal surfaces (A-B) similar to that described for minKLacZ expression in the AV bundle and left and right bundle branches (Kupershmidt, et al., 1999; Moskowitz, et al., 2004). Infrequent scattered blue cells were also observed in ventricular myocardium, surrounding coronary vessels, and in the myocardium of the pulmonary outflow tract. Intriguingly, in some species cells of the peripheral conduction system surround coronary arteries (Gourdie, et al., 1993), and the myocardium of the pulmonary outflow tract shares developmental and functional similarities with cells of the conduction system (Boukens, et al., 2009). This suggests that at least a portion of the minKCreERT2 expressing cells present outside of the canonical AV conduction system (ie AV node, bundle, and bundle branches) may have conduction system-like properties. Only very occasional, scattered blue cells were observed in the absence of tamoxifen (C-D; arrowheads), demonstrating tight control of Cre activity in this transgenic model.

To verify that the X-Gal stained cells observed by whole mount were in fact cells of the cardiac conduction system, the inventors evaluated β-galactosidase activity and molecular markers of the conduction system on adjacent frozen sections throughout the AV node, AV bundle, and bundle branches. The AV node consists of a heterogenous collection of acetylcholinesterase positive, HCN4 positive, Cx40 and Cx43 negative cells that delay the electrical impulse at the AV junction (Aanhaanen, et al., 2010; Anderson, et al., 2009; Efimov, et al., 2004; Gourdie, et al., 1993; Li, et al., 2008). Marked cells were present in a subset of HCN-4 positive, Cx40 negative, Cx43 negative AV nodal cells (A-E). The marked cells clustered in the inferior portion of the molecularly defined AV node, suggesting that Cre activity resides in the most distal portion of the AV node adjacent to the region penetrated by the Cx40 positive AV bundle. This distribution of cells within the AV node is similar to that reported for the limited contribution of Mef2cCre-derived cells to the AV node (Aanhaanen, et al., 2010), suggesting that the marked cells may represent second heart field-derived cells. The AV bundle and bundle branches rapidly conduct the electrical impulse from the AV node throughout the ventricles, and are molecularly defined by acetylcholinesterase activity, expression of HCN4 and Cx40, and exclusion of Cx43 (Aanhaanen, et al., 2010; Anderson, et al., 2009; Simon, et al., 1998; Gourdie, et al., 1993). Tamoxifen-inducible β-galactosidase activity was observed in virtually all cells of the AV bundle and bundle branches (F-O).

Embryonic minK expression exhibits a dynamic pattern throughout development (Honore, et al., 1991; Franco, et al., 2001). While B-galactosidase activity from the minK:LacZ knockin allele has been reported to be specific for the cardiac conduction system throughout embryogenesis (Kondo, et al., 2003), in situ hybridization experiments have observed endogenous minK expression throughout the embryonic myocardium, albeit with higher levels in the developing CCS and interventricular septum (Franco, et al., 2001). While the focus of the investigation was on the use of minK:CreERT2 BAC transgenic mice for recombination in the adult conduction system, the inventors also evaluated tamoxifen-inducible Cre activity during embryogenesis. Consistent with the reported distribution of endogenous minK transcripts, administration of tamoxifen at E9.5 and evaluation of marked cells at E13.5 in minK:CreERT2(+/−); ROSA26R(+/−) embryos resulted in marked cells at the crest of the interventricular septum, where the developing AV bundle is located, but also variably in the atria and ventricles, particularly the interventricular septum.

In summary, minK:CreERT2 BAC transgenic mice enable tamoxifen-inducible recombination in a subset of cells within the AV node and virtually all cells of the AV bundle and bundle branches of the adult cardiac conduction system. The temporal control of recombination, as well as its precise targeting to defined regions of the conduction system are useful for gain and loss of function studies in the conduction system.

### Example 4

### A TBX5-SCN5A Molecular Network Modulates Function of the Murine Adult Cardiac Condition System

Tbx5 is a T-box transcription factor that plays a crucial role in heart development (Hoogaars, et al., 2007). Dominant mutations in TBX5 cause Holt-Oram syndrome in humans (Basson, et al., 1997), characterized by developmental defects of the upper limb and heart and conduction system disease including age related atrioventricular conduction delay. The cardiac phenotype of Holt-Oram disease is largely recapitulated in Tbx5 heterozygous mice (Bruneau, et al., 2001). Furthermore, GWAS on CCS function have identified genetic variation near TBX5 that associates with PR and/or QRS interval variation (Holm, et al., 2010; Pfeufer, et al., 2010; Sotoodehnia, et al., 2010; Smith, et al., 2011), suggesting that Tbx5 plays a role in CCS function in the general population.

The inventors hypothesized that Tbx5 plays an essential role in the mature VCS. Efforts to identify the role of essential genes such as Tbx5 and unveil the transcriptional networks that establish and maintain mature CCS function have been hampered by the lack of selective CCS-specific in vivo molecular tools. The inventors generated a tamoxifen inducible VCS-specific Cre BAC transgenic mouse line, minKCreER^{T2}. In this study the inventors demonstrated that removal of Tbx5 from the VCS in Tbx5^{minKCreERT2} mice resulted in sudden death, slowing of conduction through the VCS, and arrhythmias including spontaneous ventricular tachycardia. It was determined that Tbx5 orchestrates a molecular network required for fast conduction in the VCS including regulation of the gap junction Cx40 and the voltage gated sodium channel Naᵥ1.5. The inventors identified a direct molecular link between Tbx5 and Scn5a, encoding Naᵥ1.5, via a Tbx5-responsive downstream enhancer sufficient to direct VCS-specific gene expression. The results identified a Tbx5-Scn5a molecular network essential for function of the mature VCS. The consistent identification of these genes in GWAS on CCS function highlights the importance of this pathway in regulating CCS function.

### Decreased Survival in Adult VCS—Specific Tbx5 Mutant Mice

The requirement for Tbx5 in the mature VCS was tested using a strategy providing normal Tbx5 gene dosage during development followed by VCS-specific deletion in mature mice. VCS-specific CreER^{T2} expression from the minKCreER^{T2} BAC transgene (Arnolds, et al., 2011) was utilized to recombine conditional (floxed) Tbx5 alleles (Bruneau, et al., 2001) in the mature VCS. A tamoxifen administration regime at 6 weeks of age inactivated Tbx5 in the mature VCS of Tbx5^{minKCreERT2} mice. VCS-selective loss of Tbx5 expression, evaluated by immunofluouresence 4 weeks following tamoxifen treatment, was observed in the atrioventricular bundle of Tbx5^{minKCreERT2} adult mice, but not Tbx5^{fl/fl} littermate controls. Consistent with the VCS selectivity of Cre activity in minKCreER^{T2} mice (Arnolds, et al., 2011), Tbx5 expression was maintained in atrial myocardium of both Tbx5^{minKCreERT2} and Tbx5^{fl/fl} mice.

VCS-specific Tbx5 mutant mice demonstrated sudden death (K). Tbx5^{minKCreERT2} adult mice demonstrated significantly increased mortality relative to Tbx5^{fl/fl} adult mice in longitudinal studies (p=0.03, logrank test). Sudden death in Tbx5^{minKCreERT2} mice occurred as early as 5 weeks post tamoxifen administration. These results demonstrate a requirement for Tbx5 in the mature VCS and prompted us to investigate the electrophysiologic consequences of VCS-specific Tbx5 knockout.

Loss of fast conduction in adult VCS-specific Tbx5 mutant mice. To determine the conduction system effects of VCS-specific Tbx5 removal, I performed conscious, ambulatory telemetry ECG analysis on Tbx5^{minKCreERT2} animals and littermate controls 4-5 weeks following tamoxifen administration. VCS-specific Tbx5 deletion caused severe conduction slowing. The PR interval, representing the period between atrial and ventricular depolarization, and QRS duration, representing the length of ventricular depolarization, and, in the mouse, early repolarization (A), were both significantly increased (B-C; Table 4). Conduction slowing was progressive, with gradual increases in the P-R and QRS intervals that plateau at approximately 4 weeks following tamoxifen administration. I therefore performed all further studies 4-5 weeks following tamoxifen administration, at which time P-R and QRS intervals had plateaued, but sudden death was not yet observed. To specifically localize the anatomic region of slowed conduction, the inventors performed invasive electrophysiologic (EP) studies in collaboration with the Patel Lab at The University of Pennsylvania. Removal of Tbx5 resulted in moderate prolongation of the A-H interval, representing conduction through the AV node and proximal His bundle, severe widening of the His deflection, and severe prolongation of the H-V interval, demonstrating slowed conduction through the His bundle, bundle branches, and Purkinje network (D-F, Table 4-5). Non-VCS function was not altered, with normal heart rate, sinus and AV node recovery times, and atrial and ventricular effective refractory periods (Table 4-5). Cumulatively, these studies establish an essential role for Tbx5 as a regulator of fast conduction in the VCS.

TABLE 4
Conduction Slowing Following Loss of Tbx5 in the VCS

| | |
|---|---|
| Tbx5^{fl} | Tbx5^{minKCreERT2} |

Ambulatory Telemetry

| | | |
|---|---|---|
| HR (bpm) | 548.91 ± 37.61 | 547.21 ± 37.77 |
| PR (ms) | 34.35 ± 1.63 | 51.77 ± 1.21** |
| QRS (ms) | 11.45 ± 0.27 | 15.33 ± 2.59* |
| N | 9 | 10 |

Electrophysiology

| | | |
|---|---|---|
| AH (ms) | 28.5 ± 2.6 | 35.3 ± 1.9** |
| H_{d} (ms) | 3.5 ± 0.6 | 5.5 ± 1.8** |
| HV (ms) | 10.8 ± 0.8 | 21.0 ± 4.1** |
| N | 6 | 6 |

Mean ± SD;
*p < 0.01,
**p < 0.001;
HR = Heart rate;
PR = PR-interval duration;
QRS = QRS-complex width;
AVB: Atrioventricular Block;
AH = AtrioHisian interval;
H_{d} = His-duration;
HV = Hisioventricular interval.

TABLE 5
Invasive EP Data Summary

| | Tbx5^{fl/fl} | Tbx5^{minKCreERT2} |
|---|---|---|
| SCL (ms) | 151.4 ± 18.4 | 148.8 ± 24.8 |
| FIR (bpm) | 401.7 ± 81.7 | 358.2 ± 67.6 |
| AH (ms) | 28.5 ± 2.6 | 35.3 ± 1.9* |
| H_{d} (ms) | 3.5 ± 0.6 | 5.5 ± 1.8* |
| HV (ms) | 10.8 ± 0.8 | 21.0 ± 4.1* |
| AVI (ms) | 42.1 ± 3.3 | 61.8 ± 4.6* |
| SNRT₁₂₀ (ms) | 203.5 ± 49.5 | 173.2 ± 39.3 |
| SNRT₁₀₀ (ms) | 184.0 ± 38.4 | 177.8 ± 46.1 |
| AVERP₁₂₀ (ms) | 56.7 ± 5.8 | 60.8 ± 6.7 |
| AVWBCL (ms) | 68.8 ± 10.3 | 84.2 ± 14.6 |
| AVN 2:1 | 53.9 ± 8.4 | 65.8 ± 12.4 |
| AERP₁₂₀ (ms) | 41.3 ± 4.8 | 35.8 ± 4.9 |
| AERP₁₀₀ (ms) | 41.3 ± 4.8 | 40.0 ± 6.1 |
| VERP₁₂₀ (ms) | 41.3 ± 7.5 | 36.7 ± 10.3 |
| VERP₁₀₀ (ms) | 43.8 ± 8.2 | 38.3 ± 9.8 |
| AT Episodes | 0 | 0 |
| Duration VT (sec) | 1.64 ± 0.9 | 23.0 ± 28.6 |
| VT CL (ms) | 53.3 ± 6.6 | 50.8 ± 11.0 |
| VT Episodes | 3 | 8^{†} |

Mean ± SD;
*p < 0.001;
^{†}p < 0.01;
AH = AtrioHisian interval;
H_{d} = His-duration;
HV = Hisioventricular interval;
AVI = Atrioventricular interval;
SNRT₁₂₀ = Sinus node recovery time following 120 ms drive train;
SNRT₁₀₀ = Sinus node recovery time following 100 ms drive train;
AERP₁₂₀ = Atrial ERP following 120 ms drive train;
AERP₁₀₀ = Atrial ERP following 100 ms drive train;
AVERP₁₂₀ = Atrioventricular ERP following 120 ms drive train;
AVWBCL = AV Wenckebach block cycle length;
AV 2:1 = AV 2:1 block cycle length;
VAWBCL = Ventriculoatrial Wenckebach block cycle length;
VERP₁₂₀ = Ventricular ERP following 120 ms drive train;
VERP₁₀₀ = Ventricular ERP following 100 ms drive train.
EP studies were performed by Fang Liu and Vic Patel at The University of Pennsylvania.

Removal of a single copy of Tbx5 did not result in detectable conduction slowing in preliminary studies 4-5 weeks after tamoxifen administration (Table 6). Very limited studies in aged animals administered tamoxifen at 6-7 weeks of age and studied 43 weeks after tamoxifen administration also did not demonstrate conduction slowing in Tbx5^{minKCreERT2/-} animals (Table 7). Based on these results, no further studies were conducted in Tbx5^{minKCreERT2/+} animals.

TABLE 6
No changes in ECG parameters in Tbx5^{minKCreERT2/+} mice studied
4-5 weeks after tamoxifen administration. Mean ± SD.

| | |
|---|---|
| Tbx5^{fl/+} | Tbx5^{minKCreERT2/+} |

Ambulatory Telemetry 4-5 weeks post TMX

| | | |
|---|---|---|
| HR (bpm) | 571 ± 18.96 | 566.79 ± 60.14 |
| PR (ms) | 33.12 ± 0.23 | 33.16 ± 1.67 |
| QRS (ms) | 11.41 ± 0.16 | 11.39 ± 0.89 |
| N | 4 | 4 |

TABLE 7
No changes in ECG parameters in Tbx5^{minKCreERT2/+} mice
studied 43 weeks after tamoxifen administration. Mean ± SD

| | |
|---|---|
| Tbx5^{fl/+} | Tbx5^{minKCreERT2/+} |

Ambulatory Telemetry 43 weeks post TMX

| | | |
|---|---|---|
| HR (bpm) | 612.78 ± 4.8 | 595.04 ± 146.38 |
| PR (ms) | 33.93 ± 0.36 | 32.92 ± 0.26 |
| QRS (ms) | 11.98 ± 0.47 | 11.46 ± 0.03 |
| N | 2 | 2 |

The autonomic nervous system can play an important role in modulating cardiac conduction. Specifically, activation of the sympathetic nervous system can increase heart rate, contractility, and accelerate conduction through the AV node, while parasympathetic stimulation via the vagus nerve can act at the SA and AV nodes to decrease heart rate and slow AV conduction. To determine whether slowed conduction in Tbx5^{minkCreERT2} mice was secondary to increased vagal tone, the inventors administered atropine, a cholinergic antagonist, to tamoxifen-treated Tbx5^{minKCreERT2} and Tbx5^{fl/fl} mice and evaluated conduction intervals by EP studies. Cholinergic blockade did not result in accelerated conduction in or Tbx5^{minKCreERT2} or Tbx5^{fl/fl} mice (Table 8), suggesting that slowed conduction in the absence of Tbx5 in the VCS was not secondary to effects on the autonomic nervous system.

TABLE 8
EP Intervals Following Atropine Administration

| | | SCL (ms) | AH (ms) | Hd (ms) | HV (ms) |
|---|---|---|---|---|---|
| Tbx5^{fl/fl} | Baseline | 157.3 ± 38.4 | 27.3 ± 9.7 | 4.0 ± 1.7 | 8.3 ± 3.0 |
| (n = 3) | Post-Atropine | 141.3 ± 30.9 | 28.0 ± 9.9 | 3.7 ± 1.5 | 9.0 ± 3.2 |
| Tbx5^{minKCreERT2} | Baseline | 130.0 ± 30.6 | 33.3 ± 5.9 | 13.7 ± 3.8 | 26.3 ± 4.8 |
| (n = 3) | Post-Atropine | 146.3 ± 11.6 | 35.7 ± 3.6 | 11.7 ± 2.5 | 26.0 ± 2.4 |

Mean +/− SD;
SCL: sinus cycle length,
AH: AtrioHisian interval,
Hd: His interval duration,
HV: Hisioventricular interval.
Cholinergic blockade studies were performed by Kurt Schillinger and Vic Patel at the University of Pennsylvania.

Cardiac arrhythmias in Tbx5minKCreERT2 mice. Removal of Tbx5 from the VCS resulted in significant arrhythmias. Observed rhythm disturbances that occurred exclusively in Tbx5^{minKCreERT2} mice included Mobitz Type II second degree AV block, indicative of defects in the His bundle and/or bundle branches (Vijayaraman, et al., 2008) (G) and spontaneous ventricular tachycardia (I-J) in ambulatory recordings. Occasional second degree AV block was observed in both Tbx5^{fl/fl} and Tbx5^{minKCreERT2} mice (Table 9). However, Mobitz type II AV block, a sign of infranodal conduction system disease characterized by one or more dropped QRS complexes without changes in the P-R interval, was observed exclusively in Tbx5^{minKCreERT2} mice. In contrast, AV block in Tbx5^{fl/fl} mice was characterized by a shortened P-R interval following the dropped beat indicating Wenkebach (Mobitz type I) AV block. Wenkebach-type AV block occurs at the level of the AV node and is generally benign and unlikely to progress to complete AV block. Premature ventricular contractions (PVCs; H) were also much more common in Tbx5^{minKCreERT2} than control Tbx5^{fl/fl} mice, with a maximum of 7 PVCs per 24 hour recording in Tbx5^{fl/fl} mice compared to greater than 100 PVCs per 24 hour recording in 6 out of 10 Tbx5^{minKCreERT2} mice.

TABLE 9
Characterization of Second Degree AV Block.

| | |
|---|---|
| Tbx5^{fl} | Tbx5^{minKCreERT2} |

Ambulatory Telemetry

| | | |
|---|---|---|
| Dropped beats/ | 5.9 ± 13.99 | 9.6 ± 15.49 |
| 24 hour recording | | |
| Mice with intermittent Mobitz Type 2 | 0 | 7 |
| 2^{nd} degree AVB | | |
| N | 9 | 10 |

Episodes of spontaneous, monomorphic ventricular tachycardia were observed in 3 of 10 Tbx5^{minKCreERT2} mice vs 0 of 9 littermate controls in ambulatory studies and 1 of 6 Tbx5^{minKCreERT2} mice vs 0 of 6 littermate controls in EP studies. In addition to the observation of spontaneous tachyarrythmias in both ambulatory monitoring (I-J) and EP studies, Tbx5^{minKCreERT2} mice showed significantly increased susceptibility to ventricular tachycardia following burst stimulation in EP studies (Table 5). Episodes of VT induced with programmed stimulation in Tbx5^{minKCreERT2} mice resembled those that occurred spontaneously but were of shorter duration. In contrast, Tbx5^{fl/fl} mice exhibited only nonsustained episodes of polymorphic ventricular tachycardia following programmed stimulation. Furthermore, the Tbx5^{minKCreERT2} mouse that developed spontaneous ventricular tachycardia during EP studies died suddenly prior to any electrophysiologic testing. Although the possibility that non-arrythmic or bradyarrythmic causes contributed to the reduced survival of Tbx5^{minKCreERT2} mice cannot be ruled out, the observation of sudden death following an episode of spontaneous ventricular tachycardia strongly suggests that ventricular tachycardia may contribute to the observed sudden death following selective removal of Tbx5 from the VCS.

Normal contractile function in Tbx5minKCreERT2 mice. To distinguish between a primary conduction system abnormality and secondary conduction system defects caused by primary contractile dysfunction, the inventors assessed cardiac contractility via echocardiography. Transthoracic echocardiography demonstrated that left ventricular function was indistinguishable between control and VCS-specific Tbx5 mutant mice (FIG. 18A-B, Table 10). Furthermore, mutant mice demonstrated immediate recovery of normal cardiac function following episodes of spontaneous ventricular tachycardia (FIG. 18C). Together these data indicate that the conduction defects observed in VCS-specific Tbx5 mutant mice did not derive from a secondary consequence of myocardial dysfunction.

TABLE 10
Left Ventricular Function is Unaffected by Loss of Tbx5 in the VCS

| | Tbx5^{fl/fl} | Tbx5^{minKCreERT2} |
|---|---|---|
| % FS | 36.46 ± 2.08 | 39.78 ± 2.10 |
| LVID (mm) | 3.69 ± 0.33 | 3.55 ± 0.23 |
| N | 5 | 5 |

Mean ± SD;
% FS: % fractional shortening;
LVID: left ventricular internal diameter.

Tbx5 is not required for survival of VCS cells. Based on the known requirement for Tbx5 for cell survival in other contexts (Goetz, et al., 2006; He, et al., 2002), it was possible that the conduction abnormalities in Tbx5^{minKCreERT2} mice may be caused by decreased survival of VCS cells. It was found, however, that the VCS fate map was not affected by deletion of Tbx5. Simultaneously deleting Tbx5 from the VCS and marking cells with the Cre-dependent lacZ reporter R26R (Tbx5^{minKCreERT2}; R26R^{minKCreERT}2/+) generated a fate map indistinguishable from that generated in the presence of Tbx5 (Tbx5^{+/+}; R26R^{minKCreERT/-}). This result demonstrates that the conduction defects in Tbx5^{minKCreERT2} mice could not be attributed to loss of VCS cells.

Tbx5 is required for VCS expression of Cx40 and Nav1.5 to modulate fast conduction. The inventors investigated whether Tbx5 is required for a functional molecular pathway mediating fast VCS conduction. The inventors analyzed the molecular basis for loss of fast conduction in VCS-specific Tbx5 mutant mice by examining expression of known mediators of fast conduction in the VCS of Tbx5^{minKCreERT2} mutant and Tbx5 control hearts. Fast conduction in the VCS requires a high degree of cell-cell electrical coupling and rapid depolarization (Kleber, et al., 2004), mediated significantly by Cx40 and Naᵥ1.5, respectively. Cx40 and Naᵥ1.5 expression were drastically reduced in the VCS following removal of Tbx5. The proximal AV bundle and distal AV bundle/bundle branches were identified by their anatomic location, acetylcholinesterase activity (Lamers, et al., 1987; Arnolds, et al., 2011; Simon, et al., 1998; van Veen, et al., 2005) and contactin-2 expression (Pallante, et al., 2010). Tbx5^{fl/fl} controls demonstrated high Cx40 and Naᵥ1.5 expression throughout the molecularly defined VCS, while Tbx5^{minKCreERT2} animals demonstrated dramatic reductions in Cx40 and Naᵥ1.5 VCS expression (C-D vs H-I and M-N vs R-S). Tbx5 was expressed in the AV bundle and bundle branches of Tbx5^{fl/fl} mice, but not Tbx5^{minKCreERT2} littermates (E vs J and O vs T). The multilead ECG recordings obtained in conjunction with the EP studies demonstrated that the spontaneous ventricular tachycardia observed in Tbx5^{minKCreERT2} mice had a left superior axis, consistent with a left posterior fasicular tachycardia (Nogami, 2010). Evaluation of the Purkinje network demonstrated that cells from the proximal (midseptal) area in Tbx5^{minKCreERT2} hearts lacked high Cx40, Naᵥ1.5, and Tbx5 expression, while cells from the distal (apical) area had high Cx40, Naᵥ1.5, and Tbx5 expression. The transition from slow to fast conduction in the Purkinje network may provide the substrate for the observed ventricular tachycardia. Furthermore, expression of HCN-4, which drives spontaneous depolarizations, was maintained in the Purkinje fibers following removal of Tbx5, and potential spontaneous depolarizations from these slow-conducting Purkinje fibers may also contribute to the observed PVCs and initiation of ventricular tachycardia in Tbx5^{minKCreERT2} mice. Consistent with the specificity of Cre activity in minKCreER^{T2} BAC transgenic mice (Arnolds, et al., 2011) and the hypothesis that slowed conduction is due to primary effects of loss of Tbx5 in the VCS, Cx40 and Naᵥ1.5 expression were not altered outside of the VCS.

Tbx5-dependent activation of an Scn5a enhancer. The inventors hypothesized that Tbx5 may directly drive a molecular network required for fast VCS conduction via direct regulation of Cx40 and Scn5a, and efforts were focused on Scn5a as no direct regulators of Scn5a in the VCS have been identified; Cx40 is a known Tbx5 target in the embryo (Bruneau, et al., 2001). The Scn5a locus was bioinformatically interrogated to identify potential Tbx5-responsive enhancers utilizing the overlap of four independent data sets: 1) evolutionary conservation (Davydov, et al., 2010); 2) ChIP-seq studies identifying Tbx5 binding sites in the atrial cardiomyocyte HL-1 cell line (He, et al., 2011); 3) p300 ChIP-seq peaks to mark active enhancers, both in vitro and in vivo (He, et al., 2011; Blow, et al., 2010); and 4) bioinformatic predictions of cardiac enhancers (Narlikar, et al., 2010. Within the genomic region including the Scn5a locus and complete upstream and downstream intergenic regions, a single region approximately 15 kb downstream of Scn5a demonstrated overlap in all four data sets. The inventors hypothesized that this genomic region was a Tbx5-dependent Scn5a enhancer.

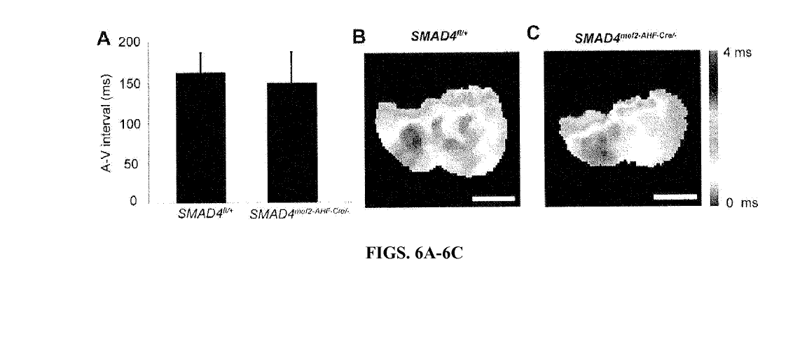

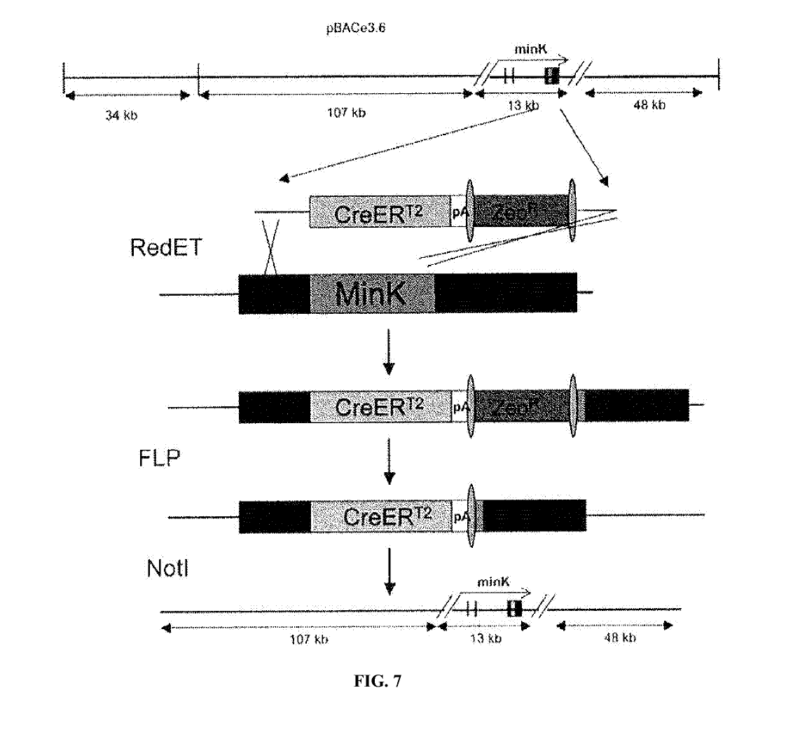

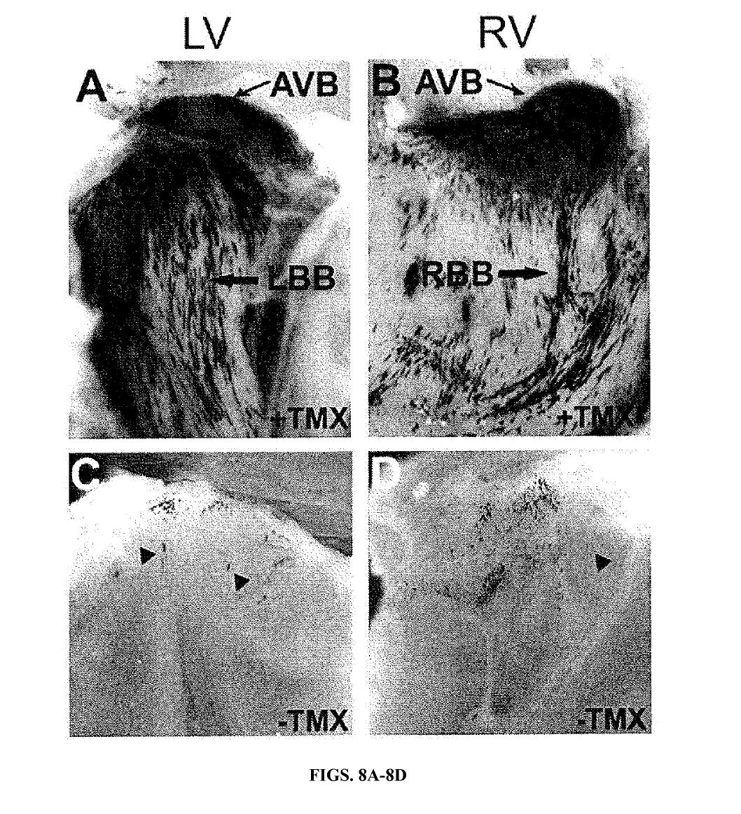

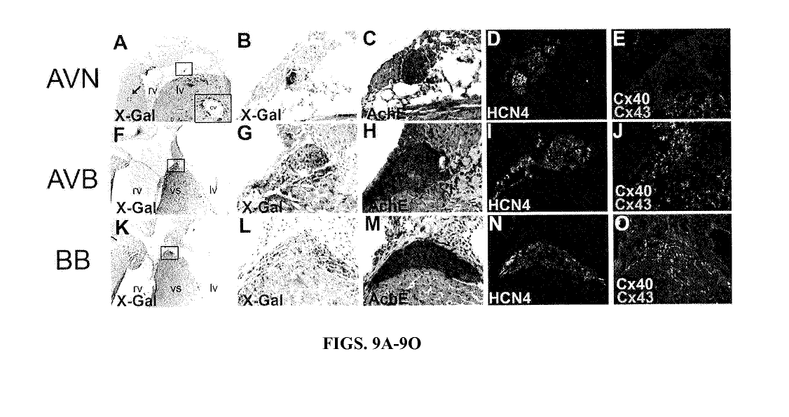

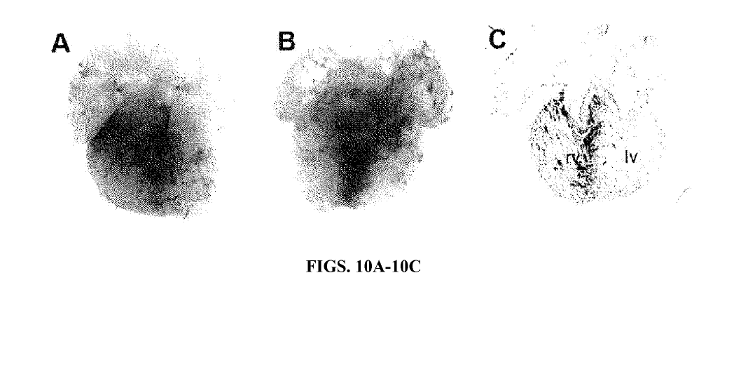

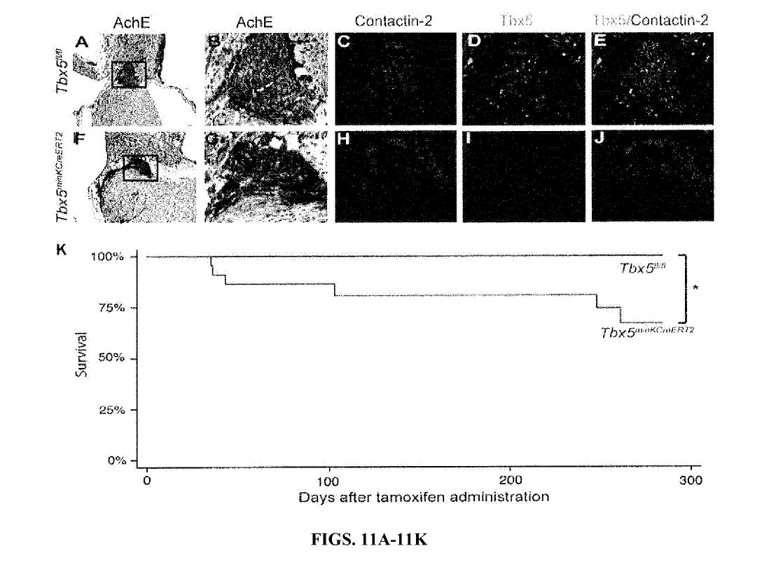

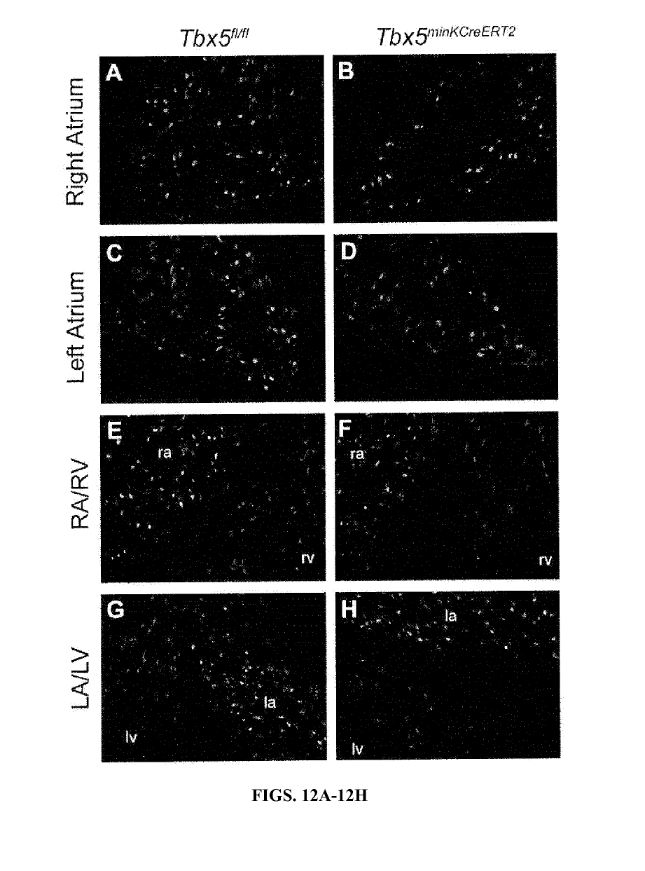

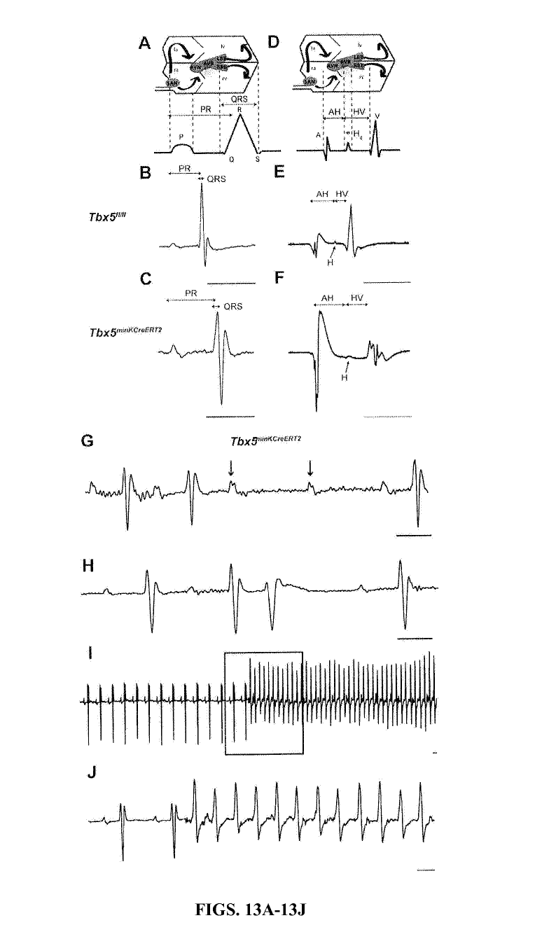

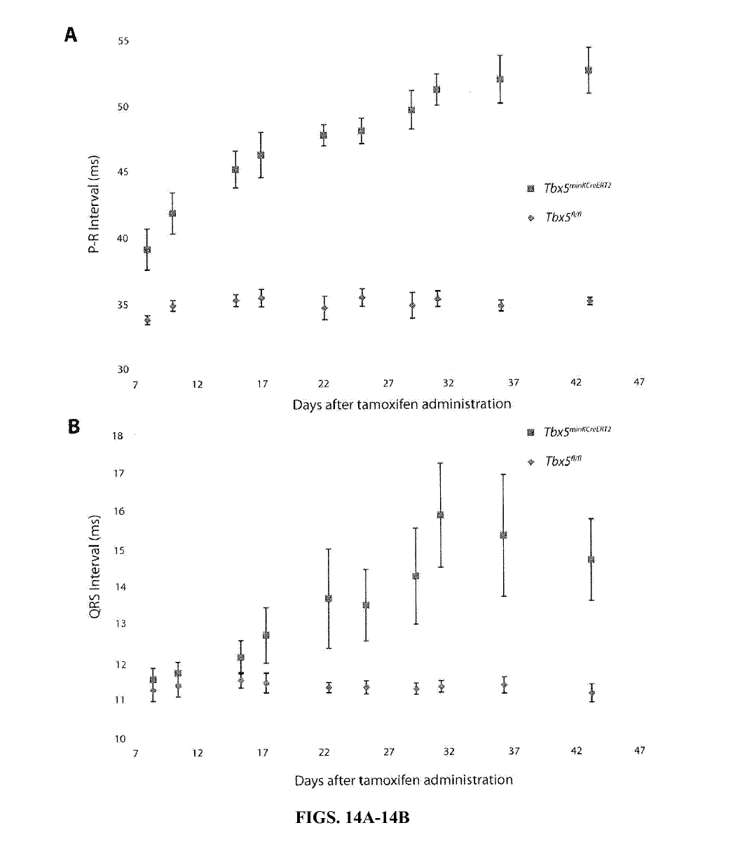

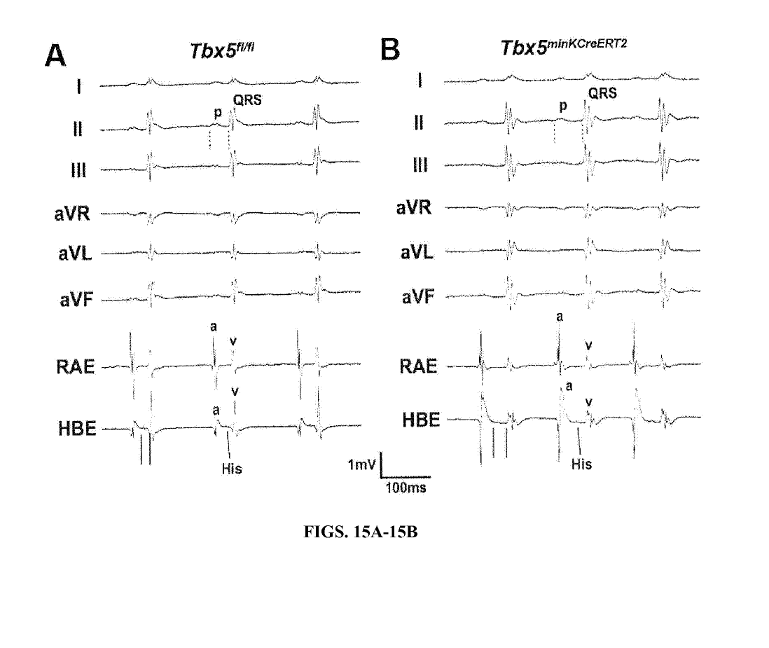

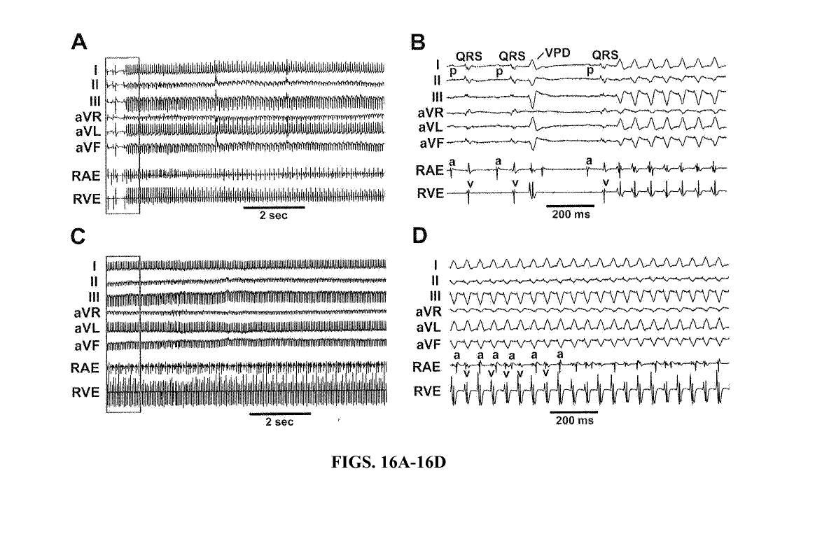

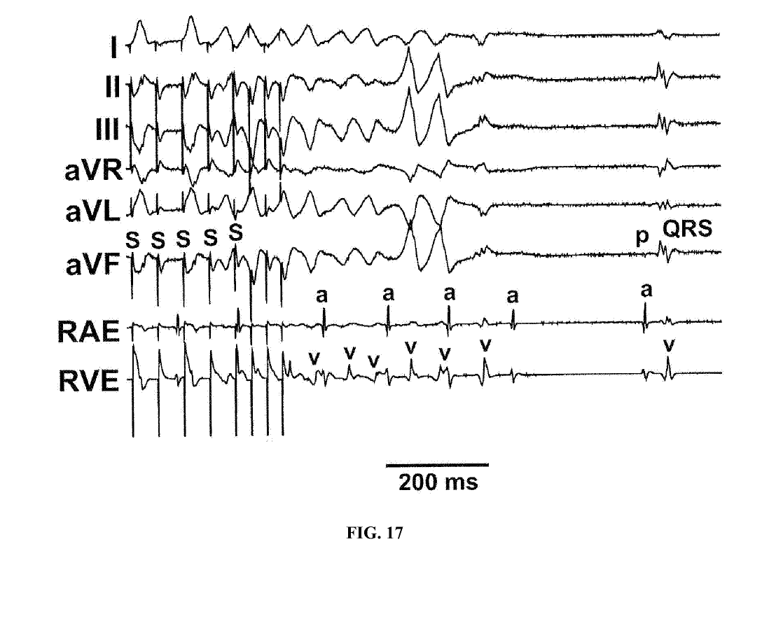

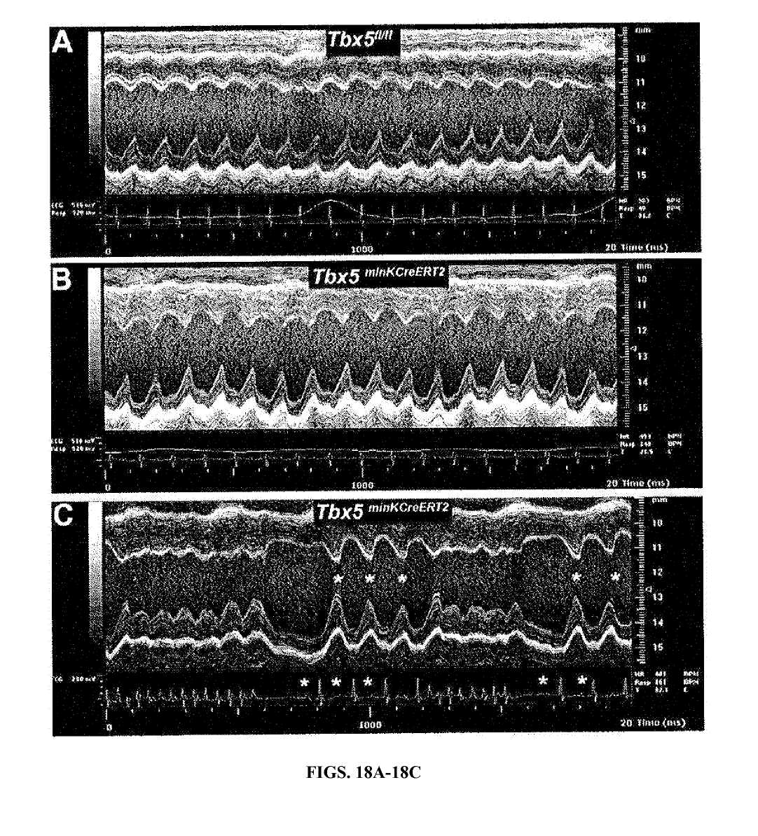

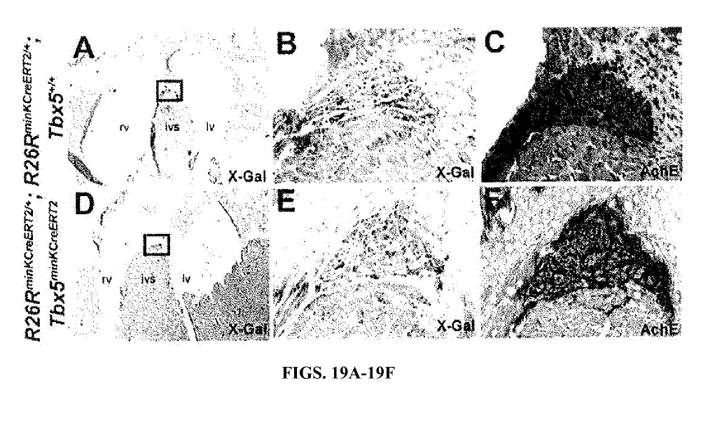

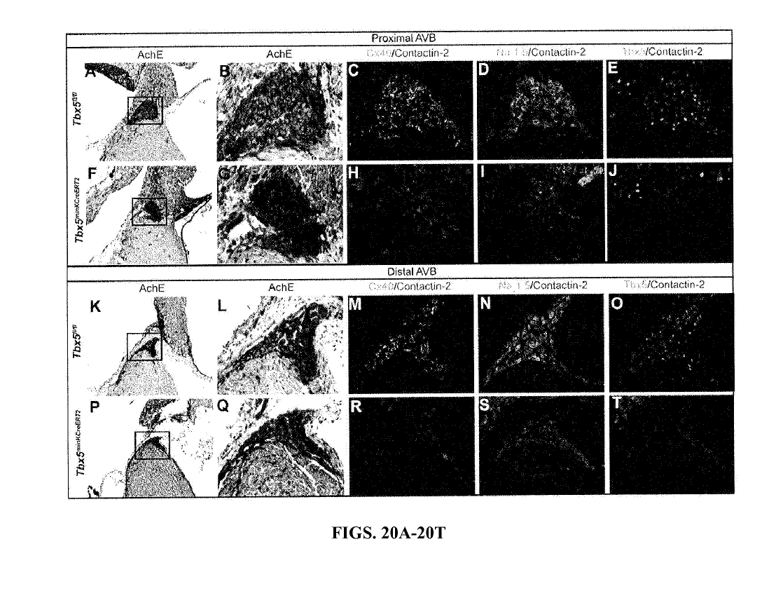

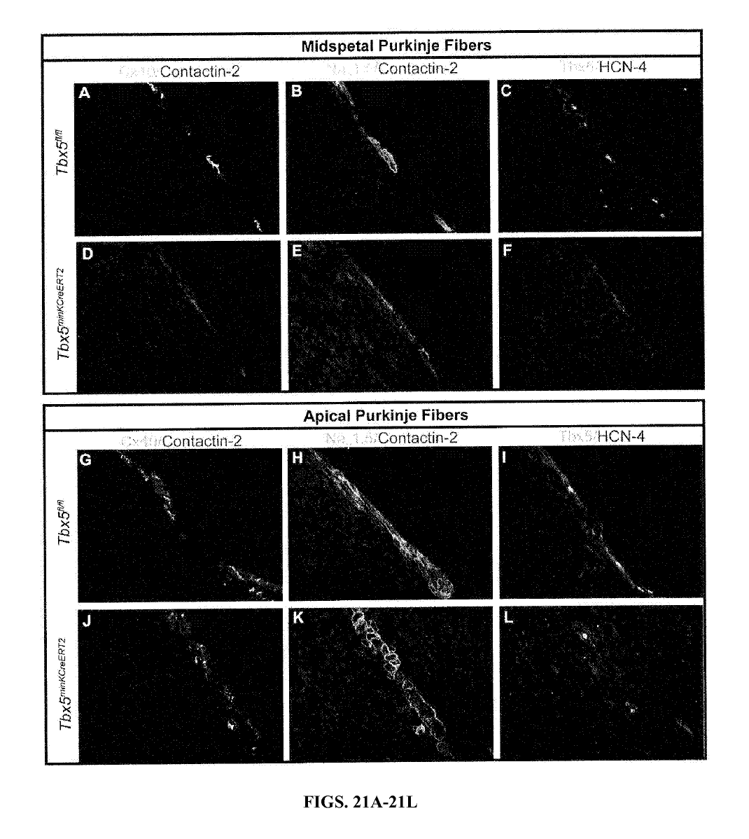

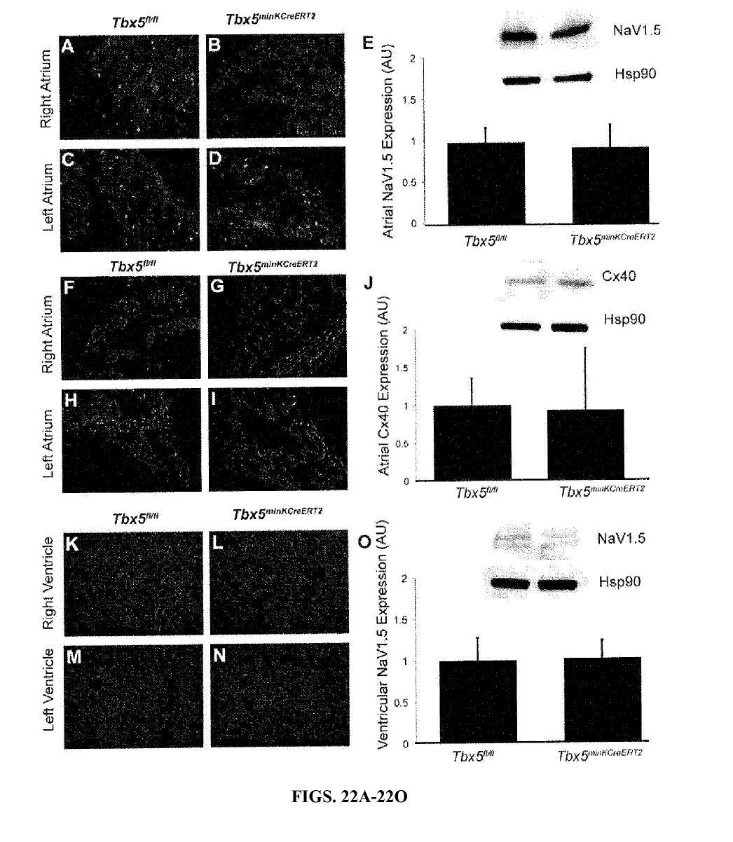

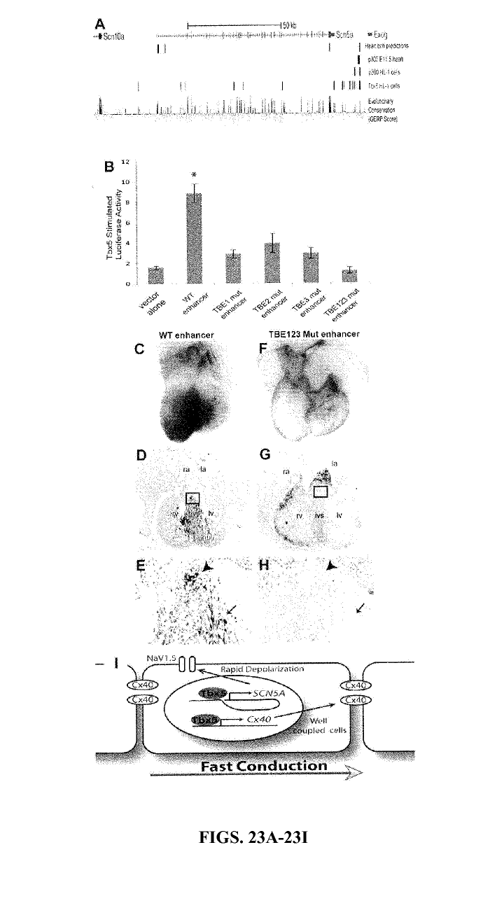

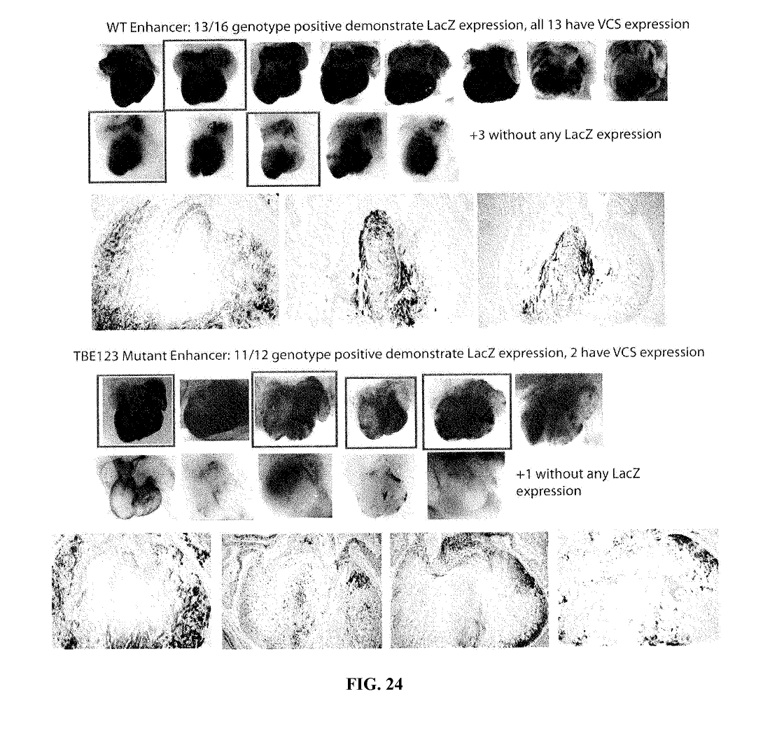

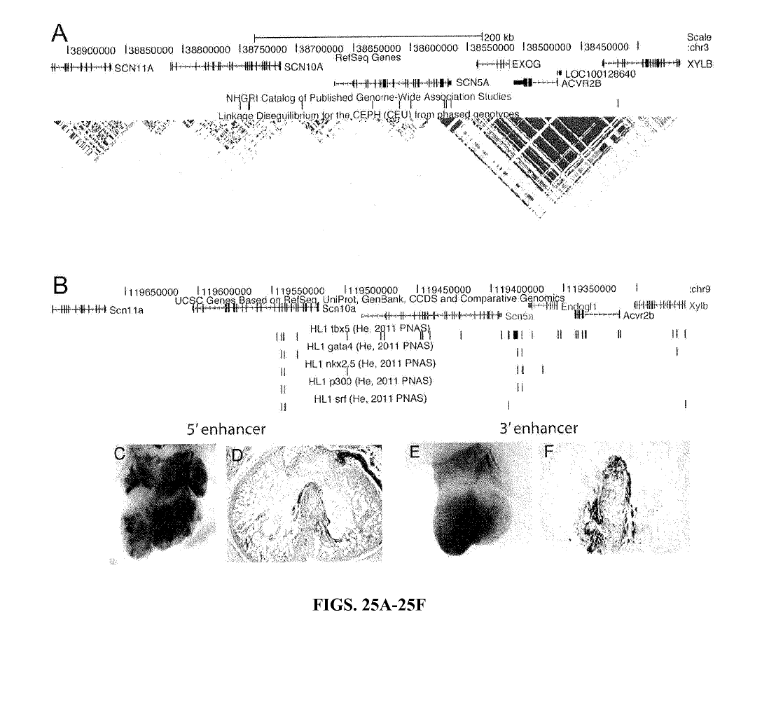

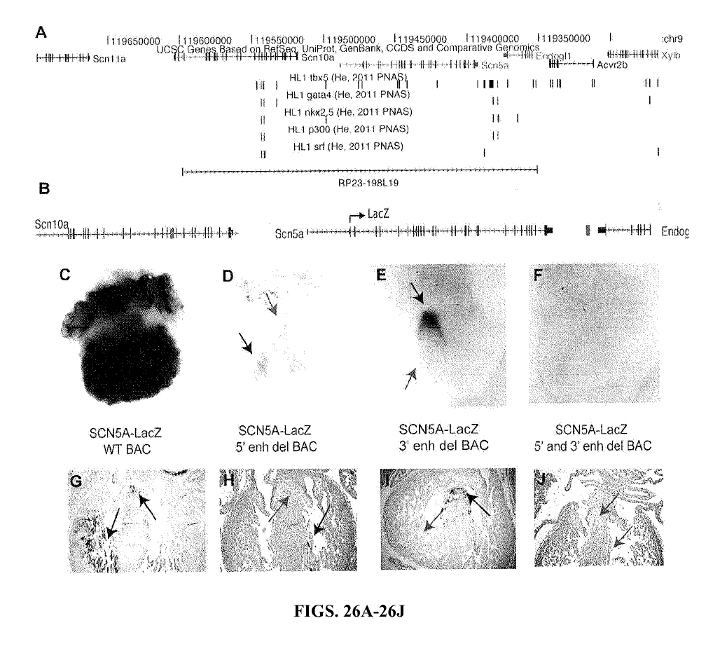

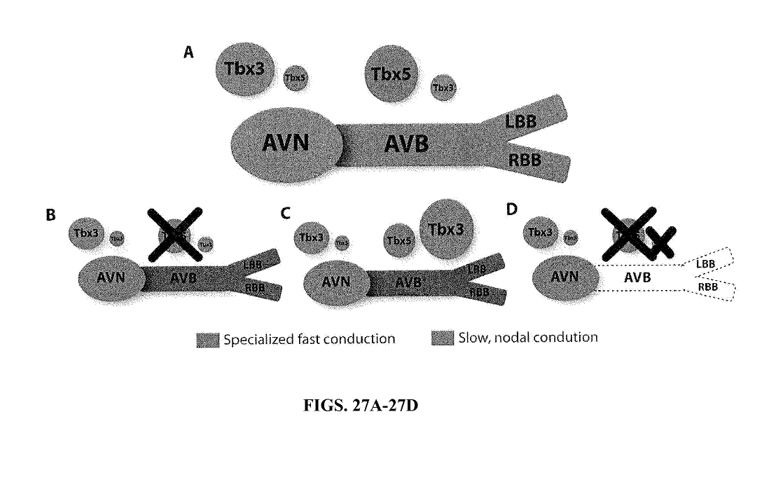

The inventors tested the ability of this putative enhancer to activate transcription in a Tbx5-dependent manner. Tbx5 dramatically upregulated enhancer-dependent luciferase reporter expression from this genomic region (chr9:119378051-119379479; NCBI build 37/mm9) in vitro (FIG. 6B). This Tbx5-responsive enhancer contains 3 conserved T-box binding sites (CAGGTGTGAGCC (SEQ ID NO. 5), chr9:119,379,020 to 119,379,031; TGGGGTGTGGAG (SEQ ID NO. 6), chr9: 119,379,008 to 119,378,997; and GGAGGTGTGAAT (SEQ ID NO. 7), chr9:119,378,918 to 119,378,929). The core of the Tbx5 binding motif is GTG (He, et al., 2011; Ghosh, et al., 2001; Mori, et al., 2006), a sequence conserved in each of the three Tbx5 binding sites present in the defined enhancer.

Individual mutation of single Tbx5 binding sites from GTG to AAA at the core of each of the three conserved Tbx5 consensus motifs significantly decreased Tbx5-mediated activation of the enhancer in vitro (B). Mutation of all three Tbx5 binding sites completely abolished Tbx5-dependent enhancer activation in vitro (B).

Tbx5-responsive activity in vitro identified this enhancer as a candidate VCS enhancer. The inventors tested whether this enhancer was sufficient for VCS-specific expression in vivo. The wild-type enhancer proved sufficient to drive in vivo VCS expression of lacZ from a minimal promoter in 13/16 transgenic embryos (C-E). Specifically, the enhancer reproducibly drove robust lacZ expression in the VCS including the atrioventricular bundle and bundle branches, as well as the dorsal wall of the atria, in an overall pattern closely resembling native Scn5a expression (Dominguez, et al., 2008; Remme, et al., 2009). The VCS-specific activity of the Scn5a enhancer was T-box dependent: VCS-specific lacZ expression was severely diminished by mutation of the 3 conserved T-box sites in the enhancer. Cardiac lacZ expression in T-box mutant transgenic embryos was weak and variable, with only 3/12 transgenic embryos demonstrating detectable VCS lacZ expression (F-H). These results indicate that Tbx5 directly regulates an enhancer downstream of Scn5a sufficient for patterning VCS-specific gene expression.

It has not been previously possible to evaluate the specific role for Tbx5 within the conduction system given its broad cardiac expression in the adult heart, its requirement during cardiac development, and the structural heart defects that frequently are associated with Tbx5 haploinsufficiency (Bruneau, et al., 2001; Basson, et al., 1994). By selectively removing Tbx5 from the adult VCS in this study, a previously unknown role for Tbx5 in the mature VCS of structurally normal hearts was identified. Removal of Tbx5 from the mature VCS results in a significant increase in mortality accompanied by arrhythmias, including ventricular tachycardia, and a dramatic slowing of conduction through the VCS. Slowed VCS conduction was manifest as AV block with dramatic increases in the H_{d} and H-V intervals as well as a prolonged QRS interval, demonstrating interventricular conduction delay. It was further demonstrated that the loss of fast conduction in the VCS was not secondary to a loss of contractile function or loss of VCS cells, but rather was associated with reductions in Cx40 and Naᵥ1.5 expression in the VCS. Tbx5 directly regulates Naᵥ1.5 expression via an enhancer downstream of the Scn5a locus that possesses T-box element dependent VCS-specific expression in vivo. These results establish the first transcriptional pathway required for function of the mature VCS and establish Tbx5^{minKCreERT2} mice as a model for the pathogenesis of ventricular conduction system disease.

### Example 4

### Genome-Wide Association Studies Preict SCN5A Enhancers

As discussed above, it was established that Tbx5 plays an essential role in the mature ventricular conduction system and identified SCN5A as a novel Tbx5 target. The Tbx5-responsive enhancer identified downstream of Scn5a is in close proximity to multiple SNPs identified in GWAS on CCS function (Holm, et al., 2010; Pfeufer, et al., 2010; Sotoodehnia, et al., 2010; Chambers, et al., 2010). This observation prompted a more thorough investigation of the hypothesis that genomic regions identified in GWAS on CCS can be combined with ChIP-Seq data from model systems to identify CCS enhancers that may be of particular relevance to CCS function in the general population.

Recent GWAS on ECG intervals in diverse populations have consistently identified variation on chromosome 3 near the SCN5A and SCN10A genes that correlates with variation in CCS function (A). Analysis of the GWAS hits in this region demonstrates that the majority fall into two distinct linkage disequilibrium blocks, one upstream of SCN5A in the SCN10A locus and one downstream of SCN5A (FIG. 1) (Holm, et al., 2010; Pfeufer, et al., 2010; Sotoodehnia, et al., 2010; Chambers, et al., 2010). . As discussed above, it was demonstrated that an enhancer downstream of SCN5A, referred to here as the 3′ enhancer, is sufficient to direct in vivo conduction system expression, and that this enhancer is activated by Tbx5 (Chapter 5). This enhancer is included in the downstream LD block. Analysis of ChIP-Seq peaks from HL-1 cells (He, et al., 2011) in the region tagged by the LD block at the SCN10A locus identified a 3.4 kb region (chr9:119540800-119544032) that demonstrated Tbx5, Gata-4, Nkx2.5, SRF, and p300 binding, suggesting that the region may possess cardiac enhancer activity (B). The inventors tested this putative enhancer for cardiac activity in vivo in transient transgenic assays. The enhancer, referred to as the 5′ enhancer, directed LacZ expression upstream of a minimal promoter in 8/8 E13.5 transient transgenic embryos, with highest activity in the developing AV bundle and trabeculae in a pattern similar to that which was previously described for the 3′ enhancer. This finding demonstrates that GWAS on CCS function predict CCS enhancers.

While enhancers can be found at great distances from their target gene, in the absence of compelling evidence to the contrary, SNPs identified in GWAS are generally presumed to act by affecting expression or function of the nearest gene. This paradigm predicts that variation in the GWAS-tagged enhancer downstream of SCN5A results in variation in CCS function by affecting SCN5A transcription levels, and that variation near the SCN10A locus affects SCN10A transcription or function. SCN5A encodes Naᵥ1.5, the alpha subunit of the major cardiac voltage-gated sodium channel, and plays a central role in cardiac physiology and pathophysiology. Mutations in SCN5A underlie numerous human disease conditions, including Brugada syndrome, Lev-Lenegre syndrome, long QT syndrome, dilated cardiomyopathy, atrial fibrillation, sick sinus syndrome, progressive cardiac conduction disease, and sudden death (Wilde, et al., 2011). There is thus a logical biological connection between variation near SCN5A and variation in ECG intervals in GWAS on CCS function. In contrast, SCN10A was not thought to play a role in the heart prior to its identification in GWAS on CCS function.

SCN10A encodes a tetrodotoxin-resistant voltage-gated sodium channel, Naᵥ1.8, with predominant expression in sensory neurons (Akopian, et al., 1996). SCN10A^{-/-} mice are viable and fertile, but have reduced sensitivity to pain induced by cold and mechanical stimuli (Akopian et al., 1999; Abrahamsen, et al., 2008; Zimmermann, et al., 2007). A hyperactivating point mutation in SCN10A results in increased sensory neuron excitability and sensitivity to cold stimuli, supporting previous loss of function experiments, and also causes tonic immobility in response to scruffing (Blasius, et al., 2012). Early reports did not detect SCN10A mRNA in heart (Akopian, et al., 1996), and LacZ expression was not detected in Naᵥ1.8 knockin mice crossed with the Cre-dependent LacZ reporter R26R (Stirling, et al., 2005). Until quite recently, Naᵥ1.8 expression was believed to be specific for sensory neurons. The consistent identification of SNPs near the SCN10A locus in GWAS on CCS function (Holm, et al., 2010; Pfeufer, et al., 2010; Sotoodehnia, et al., 2010; Chambers, et al., 2010). prompted interest in the possibility that Naᵥ1.8 may play a role in CCS function. This was a particularly appealing hypothesis given that the highest scoring SNP in one study was a non-synonymous SNP, predicted to cause an amino acid change in the intracellular domain of Naᵥ1.8 (Chambers, et al., 2010).

Functional studies on a potential role for Naᵥ1.8 in the heart are conflicting. Ambulatory ECG recordings of SCN10A^{-/-} mice demonstrated a very subtle (1.5 ms) but statistically significant decrease in the PR interval relative to wild-type littermates (Chambers, et al., 2010). This suggests that loss of SCN10A results in accelerated cardiac conduction and that the endogenous function of Naᵥ1.8 is to decrease the speed of cardiac conduction through the AV node and proximal bundle branches. Administration of A-803467, a pharmacologic inhibitor of Naᵥ1.8, resulted in increased P-R, QRS, and H-V intervals (Sotoodehnia, et al., 2010). This demonstrates a strong effect of A-803467 on conduction velocity in the His bundle. Notably, no cardiac phenotype was reported following ablation of Naᵥ1.8-expressing cells, achieved by crossing Naᵥ1.8-Cre knockin mice with a Cre-conditional diptheria toxin allele at the ROSA-26R locus (Abrahamsen, et al., 2008). Mice with hyperactivation of Naᵥ1.8 do not have baseline differences in heart rate, P-R, or QRS intervals, but do exhibit sinus bradycardia following scruffing (Blasius, et al., 2012). This effect was blocked by administration of atropine, suggesting that this effect is mediated by increased vagal stimulation at the sinus node. Consistent with a predominant role for Naᵥ1.8 in the nervous system, studies of human atrial appendage tissue suggest while Naᵥ1.8 is detectable by immunohistochemistry in atrial myocardium, the majority of Naᵥ1.8 in human atria is associated with nerve and vascular fibers (Facer, et al., 2011). With the exception of the studies using A-803467, the available evidence suggests an extremely limited, if any, role for Naᵥ1.8 in CCS function.

One potential explanation for the discordant findings using A-803467 is that this pharmacologic Na⁺ channel antagonist does not exclusively affect Naᵥ1.8 function, but rather is 100 fold more active against Naᵥ1.8 than against Naᵥ1.5 at the dose used (Sotoodehnia, et al., 2010; Jarvis, et al., 2007). While Naᵥ1.8 has been detected at the mRNA level in mouse and human hearts (Sotoodehnia, et al., 2010; Chambers, et al., 2010), the available evidence suggests that it is expressed at relatively low levels. In contrast, Naᵥ1.5 is extremely abundant in the mouse heart. Low expression of Scn10a relative to Scn5a to has been confirmed by quantitative real-time PCR (Vincent Christoffels, personal communication), as well by mRNA-Seq (Marcelo Nobrega and Scott Smemo, personal communication). This suggests that the observed conduction slowing following A-803467 administration may be due to a predominant effect on Naᵥ1.5. Furthermore, it suggests the hypothesis that GWAS identification of SNPs near SCN10A that affect CCS function act by disrupting SCN5A expression.

The inventors directly tested whether GWAS-tagged enhancers at the SCN5A and SCN10A loci affect SCN5A expression using an in vivo BAC transgenic reporter system. A mouse BAC, RP23-198L19 was identified that includes both the 5′ and 3′ enhancers, as well as the entire Scn5a coding region. LacZ was inserted into the endogeneous start site of the Scn5a locus in BAC RP23-198L19 using a recombineering strategy (Copeland, et al., 2001). 4/4 transient transgenic embryos carrying this BAC demonstrated cardiac LacZ expression, with highest expression in the developing AV bundle and trabeculae, consistent with endogenous Scn5a expression (Dominguez, et al., 2008; Remme, et al., 2009). Deletion of the 5′ enhancer resulted in dramatic reductions in cardiac LacZ expression: 9 transgenic embryos were examined and β-galactosidase activity was detectable only in a very small population of cells in the distal portion of the ventricular conduction system. Deletion of the 3′ enhancer also resulted in reduced cardiac LacZ expression: 5 transgenic embryos were examined and had reduced B-galactosidase activity relative to the WT BAC, although β-galactosidase expression was preserved in the embryonic AV bundle. Deletion of both enhancers completely ablated cardiac LacZ expression: of 7 transgenic embryos, 0 had cardiac LacZ expression. From these experiments, the following conclusions can be drawn: 1.) The 5′ and 3′ enhancers both modulate expression at the Scn5a locus. 2.) The 5′ enhancer is sufficient to drive expression in the AV bundle in the absence of the 3′ enhancer, whereas the 3′ enhancer is sufficient to drive expression in the distal VCS in the absence of the 5′ enhancer. 3.) The 5′ and 3′ enhancers are required for cardiac expression at the Scn5a locus; proximal promoter elements or other regulatory elements are not sufficient to drive cardiac expression in the absence of these enhancers.

It was further shown that Tbx5 is required for maximal Naᵥ1.5 expression in the AV bundle and that the 3′ enhancer is Tbx5-responsive. A common SNP identified in GWAS on CCS function, rs6801957, is found in the 5′ enhancer and disrupts the core of a canonical Tbx5 binding site. Based on this information, it is believed that Tbx5 also regulates Scn5a expression via the 5′ enhancer, and that disruption of the Tbx5 binding site via rs6801957 reduces expression from the Scn5a locus.

The inventors integrated GWAS hits at the SCN5A/SCN10A locus with cardiac ChIP-Seq data and identified enhancers in linkage disequilibrium with the genomic regions tagged by GWAS. Using transient transgenic assays, it was demonstrated that these enhancers are sufficient to direct in vivo CCS expression. Using an in vivo BAC transgenic reporter assay, it was demonstrated that these enhancers are necessary for transcriptional activity from the Scn5a locus in the context of a BAC sufficient to drive expression mirroring that of endogenous Scn5a. Based on consistent GWAS identification in studies on CCS function, SCN10A has been the focus of intense study for a potential role in modulating cardiac conduction. While the results do not rule out a role for SCN10A in modulating cardiac conduction, they reconcile weak and conflicting evidence for a functional role for SCN10A in the CCS with consistent identification of the locus in GWAS by suggesting that an enhancer tagged by GWAS modulates expression at the Scn5a locus.

### Discussion

minKGFP is a novel marker for the cardiac conduction system. minKGFP BAC transgenic mice were generated with the goal of creating a novel in vivo marker with complete specificity for the developing and mature cardiac conduction system. It was found, however, that GFP was not expressed exclusively in the conduction system of minKGFP hearts. Instead, GFP expression in minKGFP transgenic hearts was highest in cells of the E10.5 outflow tract, AV canal, and primary/interventicular ring. Later in development high levels of GFP expression marked the conduction system in a manner similar to that previously described for minK^{LacZ} knockin mice (Kupershmidt, et al., 1999; Kondo, et al., 2003). The minKGFP mice were useful to obtain a population of cells, minKGFP^{high}, that express molecular markers consistent with conduction system identity, thereby providing a population enriched, if not specific, for the developing CCS. Despite the heterogenous identity of minKGFP¹ cells, unbiased transcriptional profiling using E10.5 minKGFP^{high} hearts could generate testable hypotheses regarding pathways that may be important for the development of the AV bundle. The most prominent pathway that emerged from analysis of the transcriptional profiling experiments was upregulation of BMP signaling genes in minKGFP^{high} cells. While BMP signaling has been shown to play a critical role in the patterning of the AV canal and the outflow tract (Wang, et al., 2011), as well as the AV node (Stroud, et al., 2007), a potential role for BMP signaling in the development of the AV bundle had not been previously explored.

A role for BMP signaling in molecular specification of the AV bundle. The results indicate a modest role for BMP signaling in the molecular specification of the developing AV bundle. Specifically, BMP signaling genes are preferentially upregulated in E10.5 minKGFPhigh cells. Upregulation of BMP signaling genes in minKGFPhigh cells does not solely reflect the inclusion of cells of the AV canal and outflow tract in this population, as several of the genes identified exhibit specific expression in the E10.5 primary ring. Id2 and Tbx3, transcription factors that play key roles in specifying the AV bundle

(Moskowitz, et al., 2007; Bakker, et al., 2008), are downregulated in the developing AV bundle in the absence of Smad4. This establishes a role for BMP signaling in the molecular specification of the AV bundle and suggests that a hierarchy downstream of BMP signaling participates in patterning the embryonic AV bundle.

The AV bundle was not completely misspecified by removal of Smad4-dependent canonical BMP signaling. For example, Cx43, a marker for working myocardium, was not misexpressed in the AV bundle of Smad4mef2c-AHF-Cre/− embryos. Complete absence of Tbx3 results in ectopic Cx43 expression in the AV bundle (Bakker, et al., 2008; Frank, et al., 2012). As Tbx3 expression was reduced, but not eliminated, in the AV bundle of E14.5 Smad4mef2c-AHF-Cre/− embryos, the inventors believe that the residual Tbx3 expression in Smad4mef2c-AHF-Cre/− mutant embryos was sufficient to repress Cx43. Notably, reductions in Tbx3 expression must fall below a relatively high threshold before phenotypic effects are observed. For example, mice haploinsufficient for Tbx3 do not demonstrate molecular or functional conduction system disease (Bakker, et al., 2008; Frank, et al., 2012).

While the investigation revealed defects in the patterning of the embryonic AV bundle in the absence of Smad4, no resultant functional deficits in embryonic conduction system function were observed by optical mapping. Optical mapping at E12.5 has previously demonstrated normal conduction from the AV ring to the ventricular myocardium despite misspecification of the AV bundle (Bakker, et al., 2008). Specifically, hearts lacking Tbx3 were shown to have normal conduction system function by optical mapping; subsequently, reproducible arrhythmias were demonstrated by Doppler ultrasound and direct ECG recordings in vivo (Bakker, et al., 2008; Frank, et al., 2012). It is thus possible that SMAD4mef2c-AHF-Cre/− hearts have functional conduction system defects that are undetectable by optical mapping.

The AV bundle initially shares many molecular and functional characteristics with the slow-conducting primary myocardium, which also includes the embryonic AV canal and outflow tract (Moorman, et al., 2003). Unlike the AV canal, which gives rise to the slow-conducting AV node (Aanhaanen, et al., 2009), the primary ring gives rise to the rapidly conducting AV bundle and proximal branches (Miquerol, et al., 2011). Misspecification of the AV bundle by loss of Tbx3 (Bakker, et al., 2008) or Smad4 expression (Chapter 3) affects the molecular patterning of the embryonic AV bundle, but does not affect function at E12.5, as discussed above. This may reflect a predominant functional role for trabecular myocardium, which later gives rise to the Purkinje network (Miquerol, et al., 2011), in orchestrating apex-base activation of the embryonic ventricle. These results also suggest the possibility that specification of the AV bundle is an independent event from specification of the primary ring.

minKCreERT2 BAC transgenic mice are a novel tool for tamoxifen-inducible recombination in the murine adult VCS. The lack of Cre drivers with specificity for the cardiac conduction system has hampered investigations of the CCS role of genes with broad cardiac expression patterns. The inventors used a BAC recombineering approach targeting the minK locus to create an inducible Cre driver with specificity for the cardiac conduction system. During embryogenesis, tamoxifen-inducible Cre activity was not specific for the cardiac conduction system, but rather resulted in the labeling of atrial and ventricular myocytes in addition to cells of the CCS. In 6 week old animals, however, minKCreERT2 BAC transgenic mice enabled consistent, robust recombination throughout the ventricular conduction system.

A small portion of cells in the AV node also exhibited tamoxifen-inducible Cre activity, as did occasional, scattered atrial and ventricular myocytes and cells surrounding coronary arteries. Based on their location in the inferior portion of the AV node, the inventors hypothesize that the minKCreERT2-labeled cells in the AV node may represent those that form the extension of the AV bundle into the AV node. These cells may overlap with the small population of Cx40 negative cells labeled by the Mef2AHFCre transgene, which labels the AV bundle, but not the majority of the AV node (Aanhaanen, et al., 2010), although this has yet to be established definitively. The identification of markers specific for this population or 3D reconstructions of this domain could contribute to a greater understanding of the identity of the Cx40 negative cells with tamoxifen-inducible Cre activity in minKCreERT2 BAC transgenic mice. Tamoxifen-inducible Cre activity was also found in cells surrounding coronary vessels; in some species cells of the peripheral conduction system surround coronary arteries (Gourdie, et al., 1993). This suggests that at least a portion of the minKCreERT2 expressing cells present outside of the canonical AV conduction system (ie AV node, bundle, and bundle branches) may have conduction system-like properties.

Differences in CCS-specificity between minkGFP and minKCreERT2 BAC transgenic mice. The increased specificity for the conduction system in minKCreERT2 BAC transgenic mice relative to minKGFP BAC transgenic mice may reflect multiple mechanisms. Circular BAC DNA was used to create minKGFP BAC transgenic mice, while a linearized BAC (lacking a 34 kb fragment 107 kb upstream of minK as a result of an internal NotI site) was used to create minKCreERT2 BAC transgenic mice, and it is possible, although unlikely that this contributes to the differential specificity observed. While BAC transgenics are less susceptible to position effects than are traditional transgenics, copy number, BAC integrity, and integration site nonetheless can play an important role in determining the pattern of transgene expression. Indeed, while all 4 minKCreERT2 BAC transgenic lines examined in preliminary experiments demonstrated tamoxifen-inducible Cre activity in the VCS, the specificity and sensitivity varied between lines, likely reflecting such effects. Furthermore, detecting tamoxifen-inducible Cre activity requires sufficient protein to be present in the cell such that a pulse of tamoxifen enables translocation to the nucleus and recombination of the target locus. Tamoxifen-inducible Cre activity is thus a less sensitive detection method than is direct evaluation of GFP fluorescence.

Investigation of GFP fluorescence in minkGFP BAC transgenic mice also was concentrated at embryonic stages, whereas the evaluation of minKCreERT2 BAC transgenic mice was most thorough in adult mice. Endogenous minK expression increases during embryonic development, peaks around 7 days postnatally, and then declines in adult mice (Felipe, et al., 1994). Similarly, the in situ hybridization signal for minK has been reported to be more robust during embryonic stages than in adult mice, although it labels all cardiomyocytes at both stages (Honore, et al., 1991). Therefore, the increased specificity for the CCS in adult stages in minKCreERT2 BAC transgenic mice may reflect reduced transcriptional activity at the locus in adult mice, coupled with the decreased sensitivity of tamoxifen induction for detection. The dynamic expression of minK suggests that the age at which Cre activity is induced with tamoxifen may play a significant role in the population of cells that are labeled. Therefore induction of Cre activity in this line at ages other than that described (5 days of tamoxifen at 6-7 weeks of age) should be preceded by careful documentation of labeled cells at the age studied.

The minK locus was originally chosen in the attempt to drive CCS-specific gene expression based on studies showing that the distribution of β-galactosidase activity in minKLacZ knockin mice was specific for the conduction system (Kupershmidt, et al., 1999; Kondo, et al., 2003). The reason for the discrepancy between the reported endogenous expression of minK and the distribution of β-galactosidase activity in minKLacZ knockin mice is unclear, although it may be related to genetic perturbations at the locus as a result of the targeting vector or the sensitivity of X-Gal for detecting very low levels of LacZ expression. Cumulatively, a review of the expression patterns reported for endogenous minK transcripts, β-galactosidase activity in minKLacZ knockin mice, GFP expression in minKGFP BAC transgenic mice, and tamoxifen-inducible Cre activity in minKCreERT2 mice suggests that the transcriptional control elements surrounding the minK locus direct expression throughout cardiomyocytes, with highest activity in the ventricular conduction system, and that age and embryonic-stage specific effects modulate the intensity of expression. Regardless of the precise reasons for the differences in specificity, both minKGFP and minKCreERT2 BAC transgenic mice proved to be useful tools. The inventors took advantage of tamoxifen-inducible VCS-specific Cre activity in adult minKCreERT2 BAC transgenic mice to investigate the role of Tbx5 in the mature VCS.

Tbx5 is required for fast conduction in the mature ventricular conduction system. It has not been previously possible to evaluate the specific role for Tbx5 within the conduction system because of its broad cardiac expression in the adult heart, its requirement during cardiac development, and the structural heart defects that frequently are associated with Tbx5 haploinsufficiency (Bruneau, et al., 2001; Basson, et al., 1994). By selectively removing Tbx5 from the adult VCS, a previously unknown role for Tbx5 in the mature VCS of structurally normal hearts was identified. Specifically, removal of Tbx5 from the mature VCS resulted in a significant increase in mortality accompanied by arrhythmias, including ventricular tachycardia, and a dramatic slowing of conduction through the VCS. Slowed VCS conduction was manifest as AV block with dramatic increases in the Hd and H-V intervals as well as a prolonged QRS interval, demonstrating interventricular conduction delay. Loss of fast conduction in the VCS was not secondary to a loss of contractile function or loss of VCS cells, but rather was associated with reductions in Cx40 and Nav1.5 expression in the VCS. It was shown that Tbx5 directly regulates Nav1.5 expression via an enhancer downstream of the Scn5a locus that possesses T-box element dependent VCS-specific expression in vivo. The results establish the first transcriptional pathway required for function of the mature VCS and establish Tbx5minKCreERT2 mice as a model for the pathogenesis of ventricular conduction system disease.

A novel role for Tbx5, a congenital heart disease (CHD) gene, was identified in the adult heart, implicating a developmental transcription factor in the molecular pathways that coordinate function in the mature heart. In elderly patients with congenital heart disease in the CONCOR registry, arrhythmias have been reported in over 50% of patients (van der Bom, et al., 2011). Although some portion of this is likely secondary to factors such as structural abnormalities and surgical complications, the study demonstrates that CHD genes including Tbx5 are not limited to coordinating cardiac morphogenesis but also may play a broader role in coordinating mature heart function. This paradigm may be particularly relevant to study of the conduction system in light of the genetic variation near a large number of developmental genes identified in GWAS on PR and QRS intervals (Arnolds, et al., 2011).

No change in conduction intervals in Tbx5minKCreERT2/+ mice. In limited studies, no changes were observed in PR or QRS intervals following removal of 1 copy of Tbx5 from the VCS. In contrast, germline heterozygosity for Tbx5 consistently results in CCS abnormalities. Specifically, Tbx5+/− mice exhibit first and second degree AV block, and some but not all studies demonstrate prolongation of the QRS interval (Bruneau, et al., 2001; Mowkowitz, et la., 2004). Tbx5+/− mice demonstrate prolongation of the A-H, but not the H-V, interval by EP (Moskowitz, et al., 2004). Cumulatively these results suggest a predominant effect on conduction through the AV node, as opposed to the His bundle and bundle branches. As minKCreERT2 directs recombination predominantly in the VCS, these results may suggest increased dosage sensitivity for Tbx5 in the AV node compared to the AV bundle and bundle branches. Alternatively they may reflect differences in dosage sensitivity during development and in the mature CCS. This could be tested directly using inducible Cre drivers specific for nodal elements of the CCS, such as HCN4:CreERT2 (Hoesl, et al., 2008). Finally, recombination efficiency may contribute to the lack of effect following removal of 1 copy of Tbx5 with minKCreERT2.

Tbx5 regulation of Cx40 and Nav1.5 in the VCS. It was demonstrated that Tbx5 serves as an essential regulator of VCS function and is required for Cx40 and Nav1.5 expression in the VCS. Cx40, a high conductance gap junction expressed in the atria and VCS, mediates tight cell-cell coupling (van Veen, et al., 2001) and Cx40 knockout mice demonstrate functional VCS slowing similar to, but less severe than, Tbx5 VCS-specific knockout mice (Bevilacqua, et al., 2000; Hagendorff, et al., 1999; Simon, et al., 1998; Leoni, et al., 2010; Papadatos, et al., 2002; Tamaddon, et al., 2000; VanderBrink, et al., 2000; Verheule, et al., 1999). Neither spontaneous ventricular tachycardia nor sudden death, both observed in the Tbx5minKCreERT2 mice, have been reported in Cx40 knockout mice. The observation that Tbx5 is required for expression of Cx40 in the adult VCS extends previous work demonstrating that Cx40 is a Tbx5 target in the embryonic heart (Bruneau, et al., 2001). Previous work on the regulation of Cx40 by Tbx5 has focused on the proximal promoter. It is now known that more distal enhancer elements play essential roles in modulating gene expression. A 1 kb region (chr3:96,866,475-96,867,469) located 10 kb downstream of the Cx40 locus has binding sites for Tbx5, Nkx2-5, p300, and SRF in HL-1 cells (He, et al., 2011) and p300 binding in E11.5 hearts (Blow, et al., 2010) suggesting that it may function as a Cx40 enhancer. Future studies to characterize the necessity and Tbx5-responsiveness of this putative enhancer may be fruitful to better understand regulation of the Cx40 locus.

Rapid depolarization in the VCS is mediated by high expression of the voltage-gated sodium channel Nav1.5 (Remme, et al., 2009; Papadatos, et al., 2002). Mutations in Nav1.5, as well as alterations in its expression levels, are associated with numerous human cardiac disease conditions, yet regulation of its conduction system expression has been unknown (Rook, et al., 2012). Scn5a haploinsufficiency in mice causes conduction slowing in the VCS (Papadatos, et al., 2002) and on occasion spontaneous ventricular arrythmias including ventricular tachycardia (Leoni, et al., 2010). Tbx5 is required for high levels of Nav1.5 expression in this VCS and that Nav1.5 is directly regulated by Tbx5 via a downstream enhancer sufficient to direct VCS expression. Given that cell-cell electrical coupling and excitability are key parameters governing conduction velocity, the reductions in Cx40 and Nav1.5 expression following removal of Tbx5 are likely responsible for a significant component of the observed conduction slowing.

The relevance of the molecular network downstream of Tbx5 that was identified may not be limited to the role described in the VCS. Atrial fibrillation is the most common sustained cardiac arrhythmia, and its incidence is rapidly increasing (Mayasaka, et al., 2006). Tbx5, Cx40, and Scn5a are all expressed in the atria as well as in the fast ventricular conduction system, and mutations in CX40, SCN5A, and TBX5 are all known causes of atrial fibrillation (Wakili, et al., 2011). Furthermore, the Tbx5-responsive Scn5a enhancers identifed are active in the atria as well as the VCS. SNPs near Tbx5 have been identified in GWAS that contribute to the risk of atrial fibrillation (Holm, et al., 2010) suggesting that this pathway may be of broad relevance to the general population. It is expected that the molecular pathway identified as a key regulator of conduction in the fast ventricular conduction system is also operating in the atria and may be involved in the pathogenesis of atrial fibrillation

Strategies to identify additional Tbx5 targets in the ventricular conduction system. The inventors investigated Tbx5-dependent Cx40 and Nav1.5 expression in the VCS based on the essential and established roles of Cx40 and Nav1.5 in facilitating rapid cardiac conduction. While downregulation of Cx40 and Nav1.5 in the absence of Tbx5 likely underlies much of the observed conduction slowing in the absence of Tbx5, Cx40 and Nav1.5 are unlikely to represent the only physiologically relevant Tbx5 targets in the VCS. Future investigations into additional Tbx5 targets can draw from candidate-gene as well genome-scale approaches. Previous microarray studies have characterized embryonic gene expression in a Tbx5 allelic series (Mori, et al., 2006), as well as genes induced by adenoviral expression of Tbx5 in cell culture (Plageman, et al., 2006), although the results of these studies may have limited relevance to the role of Tbx5 in the VCS. The only ion channels identified in these studies with known cardiac expression, for example, were Cx40 (discussed at length above), Kcna5, Cacna2d2, Ttyh3, and Slc5a1. Kcna5 encodes a voltage sensitive K+ channel that is involved in generating the ultra-rapid component of the delayed rectifier K+ current and is highly expressed in atria relative to ventricles (Grant, et al., 2009). Like Cx40 and SCN5A, KCNA5 has been linked to atrial fibrillation (Wakili, et al., 2011). The role of Kcna5 in the ventricular conduction system is unclear, however, and KCNA5 has been reported to be downregulated in the His bundle and Purkinje fibers from human samples relative to atrial myocardium (Gaborit, et al., 2007; Greener, et al., 2011). Cacna2d2 encodes a subunit of a voltage-gated Ca++ channel with predominant expression at the mRNA level in the SA and AV nodes (Marionneau, et al., 2005). Cacna2d2 knockout mice exhibit a trend towards bradycardia, but do not have alterations in other ECG parameters (Ivanov, et al., 2004). Ttyh3 encodes a Ca++-activated Cl— channel; while it is expressed in the heart, little is known about its distribution within the heart or its potential function (Suzuki, et al., 2006). Slc5a1 encodes a sodium-glucose co-transporter with cardiac expression, but again little is known about its role in the heart (Zhou, et al., 2003; Banerjee, et al., 2009).

Promising candidate Tbx5 targets with functional roles in the VCS can be mined from studies on genes repressed by Tbx3 overexpression (Hoogaars, et al., 2007; Bakker, et al., 2012). Tbx3 is closely related to Tbx5, and they frequently antagonistically regulate gene expression (Greulich, et al., 2011). Genes repressed by Tbx3, particularly in the atria where Tbx5 expression is high, are thus potential Tbx5 targets. The potential utility of this intellectual framework is highlighted by the finding that Cx40 and Scn5a are downregulated following Tbx3 overexpression (Hoogars, et al., 2007; Bakker, et al., 2012). Of the numerous genes affected by Tbx3 overexpression (Hoogars, et al., 2007; Bakker, et al., 2012). , Kcnk3/TASK-1, Ryr2, and Kcnj2 (Kv1.2), are notable for their known functional roles in the ventricular conduction system. Kcnk3/TASK-1 encodes a K+channel that becomes restricted to the VCS during development (Graham, et al., 2006). Knockout of TASK-1 in mice results in prolongation of the QRS interval, suggesting delayed conduction through the ventricular conduction system and ventricular myocardium (Decher, et al., 2011), as well as Q-T interval prolongation (Decher, et al., 2011; Donner, et al., 2011). Ryr2 encodes the ryanodine receptor, a Ca++ channel with a pivotal role in cardiomyocytes. Mutations in Ryr2 in humans cause catecholaminergic polymorphic ventricular tachycardia, and studies in mouse models have demonstrated a pivotal role for Purkinje cells in the initiation of these arrhythmias (Cerrone, et al., 2009). Mutations in Ryr2 have also been associated with atrioventricular conduction delay (Bhuiyan, et al., 2007); cumulatively these studies suggest a potential role for Ryr2 in maintenance of normal cardiac conduction. Kcnj2/Kv1.2 encodes a K+ channel involved in generating the inwardly rectifying K+ current that drives cardiac repolarization. Mutations in Kcnj2 cause Andersen-Tawil syndrome (Plaster, et al., 2001), a complex developmental disorder characterized by periodic paralysis, dysmorphic features, and cardiac conduction abnormalities. The ECG abnormalities in Andersen-Tawil syndrome are highlighted by characteristic T-U wave patterns. Some patients also demonstrate frequent PVCs, ventricular tachycardia, AV block, and bundle branch block, suggesting a role for Kcnj2 in the VCS (Zhang, et al., 2005). Investigation of these putative Tbx5 targets in the VCS holds the promise of establishing a deeper understanding of the molecular network downstream of Tbx5 that maintains fast conduction in the VCS.

In addition to the candidate gene strategies described above, unbiased transcriptional profiling of the VCS in the presence and absence of Tbx5 is likely to identify a large cohort of Tbx5-regulated genes in the VCS. Potential strategies to obtain VCS tissue include laser capture microdissection (LCM) or gross microdissection of the AV bundle from Tbx5minKCreERT2 and Tbx5fl/fl controls. While LCM is capable of precisely dissecting complex structures, such as the AVB, it yields relatively small amounts of tissue. Obtaining sufficient tissue for transcriptional profiling is thus a challenge with this technique. Gross microdissection is likely to increase the yield of tissue, albeit at the cost of reduced specificity. Expression profiling of ion channels from mouse, rabbit, and human hearts has been performed to obtain ion channel profiles for the atria, ventricles, AV node, bundle, and Purkinje fibers (Gaborit, et al., 2007; Greener, et al., 2011; Marionneau, et al., 2005; Greener, et al., 2009). These studies identified detailed portraits of ion channel expression, but also demonstrate the need for careful follow-up from such studies and the potential difficulties introduced by imprecise sampling. Nav1.5, for instance, has been reported to be expressed at higher levels in ventricular myocardium than the AV bundle or Purkinje fibers (Gaborit, et al., 2007; Greener, et al., 2009), despite clear evidence of higher expression in the VCS relative to ventricular myocardium at both the protein and RNA level (Remme, et al., 2009). The magnitude of expression differences also tends to be compressed by such approaches: Cx40, for instance clearly exhibits specific expression in the atria and fast ventricular conduction system by immunohistochemistry, yet ion channel profiling suggests only 3-5× enrichment in atrial relative to ventricular tissues (Gaborit, et al., 2007; Greener, et al., 2011). Despite these caveats, expression profiling on grossly dissected tissue clearly has power to identify differences in expression levels. To provide more precise gross dissections of VCS tissue, the inventors crossed the ROSA26R-YFP allele (Srinivas, et al., 2001) into the minKCreERT2 line, as well as into minKCreERT2(+/−); Tbx5(fl/fl) mice, which should allow more precise identification of the VCS (via yellow fluorescence following Cre activation). Regardless of the approach used, transcriptional profiling is likely to identify novel Tbx5 targets in the VCS and contribute to expanding the molecular network downstream of Tbx5 in the VCS.

Dissecting regulation of Scn5a by Tbx5 and uncovering molecular logic underlying GWAS on CCS function. Recent CCS GWAS have uncovered numerous loci linked to cardiac conduction function in the general population (Holm, et al., 2010; Pfeufer, et al., 2010; Sotoodehnia, et al., 2010; Chambers, et al., 2010; Smith, et al., 2011; Smith, et al., 2009). An ongoing challenge is to identify the source of functional variation tagged by GWAS. Given that GWAS have consistently identified genetic variation at the SCN5A/SCN10A locus that correlates with CCS function (Holm, et al., 2010; Pfeufer, et al., 2010; Sotoodehnia, et al., 2010) and that Scn5a were established as a Tbx5 target in the VCS, the inventors analyzed Scn5a regulatory elements to determine if they are reflected in GWAS on CCS function. Analysis of the SCN5A/SCN10A locus identified 2 enhancers, one downstream of SCN5A and one in the SCN10A locus, that are in linkage disequilibrium with the genetic regions that correlate with variation in CCS function in GWAS. Both of these enhancers are sufficient to drive VCS expression in vivo, and it was demonstrated that the downstream enhancer requires conserved T-box elements for this activity. One of the SNPs identified at the SCN10A locus disrupts a canonical T-box binding site, and ongoing studies are examining the functional effects of this mutation for Tbx5-mediated activation of the enhancer. It was further demonstrated that both enhancers are required for Scn5a expression using a BAC transgenic reporter. This is particularly noteworthy for the enhancer located in the SCN10A locus, as it reconciles weak and conflicting evidence for a role for Nav1.8, the SCN10A gene product, with the consistent identification of the region in GWAS on CCS function by demonstrating that the enhancer tagged in GWAS affects Scn5a expression.

While Nav1.5 is expressed at highest levels in the VCS, it also plays a role throughout the myocardium, and its expression varies both transmurally in the ventricles and regionally within the CCS (Remme, et al., 2009). A complete picture of the factors regulating SCN5A expression therefore must account for the full range of its cardiac expression, not just its expression within the VCS. Notably, removal of the two enhancers identified in this work results in complete absence of cardiac expression at the Scn5a locus, suggesting that regulatory elements in these enhancers may contain the cis acting elements necessary to achieve regional variation in Nav1.5 expression. Nav1.5 is excluded from the slow conducting SA and AV nodes (Remme, et al., 2009), areas with high levels of Tbx3 expression. Tbx3 is a potent transcriptional repressor capable of repressing Nav1.5 (Hoogaars, et al., 2007), which may underlie exclusion of Nav1.5 from the nodes. This indicates that Tbx5 and Tbx3 are key determinants of fast and slow conduction in the CCS. Perinatal removal of Nkx2-5 results in loss of Nav1.5 in the ventricles, while Nav1.5 expression is preserved in the atria and VCS (Briggs, et al., 2008), areas with high levels of Tbx5 expression. Both of the enhancers identified in this study are bound by Nkx2-5 in HL-1 cells (He, et al., 2011), further suggesting a role for Nkx2-5 in regulating SCN5A transcription, particularly in the ventricles. An attractive candidate transcription factor that may contribute to regulating transmural variation in Nav1.5 expression is Irx5, whose transmural expression gradient mirrors that of Nav1.5 (Christoffels, et al., 2000; Constantini, et al., 2005; Remme, et al., 2009). Loss of Irx5 affects ventricular repolarization (Constantini, et al., 2005), although an effect on Nav1.5 expression has yet to be investigated.

Molecular logic of CCS function: A Tbx5-Tbx3 code in regional specification of the atrioventricular conduction system. It was demonstrated that Tbx5 plays a crucial role in maintaining fast conduction in the VCS by driving a molecular network that includes direct regulation of Cx40 and Scn5a. Tbx3 represses Cx40 and Scn5a in vivo and is capable of activating a functional and molecular nodal phenotype (Hoogaars, et al., 2007; Bakker, et al., 2012). Tbx3 is expressed throughout the AVCS (Hoogaars, et al., 2004; Aanhaanen, et la., 2010), although Tbx5 activity appears to be dominant in the VCS. Cumulatively these results indicate that Tbx5 and Tbx3 are key determinants of regional identity in the AVCS.

It was further demonstrated that loss of Tbx5 results in a loss of fast conduction in the VCS. The model predicts that in the absence of Tbx5, Tbx3 activities will predominate, which may transform the AVB into a nodal-like structure. This may be confirmed by directly measuring ionic currents in VCS cells in the presence or absence of Tbx5 to determine if loss of Tbx5 results in a transformation to a nodal phenotype at the single cell level and determining if the AVB adopts a nodal molecular profile in the absence of Tbx5. Isolation of cells for patch clamp experiments to directly measure action potentials and ionic currents will be facilitated by using mice harboring the ROSA-YFP reporter, discussed above. Molecular evaluation may be achieved using established markers of the AVN, such as Cx30.2 (Kreuzberg, et al., 2006), ion channel profiles of the AVN identified by microdissection (Gaborit, et al., 2007; Greener et al., 2011; Marionneau, et al., 2005; Greener, et al., 2009) as well as published expression profiles of BACTbx3-EGFP hearts that express EGFP exclusively in the AVN (Horsthuis, et al., 2009). The model predicts that overexpression of Tbx3 in the AVB will result in a similar phenotype to removal of Tbx5. One approach to demonstrating this is to cross a Cre-conditional Tbx3 overexpression line (Hoogaars, et al., 2007) with minKCreER^{T2} mice. Finally, the model predicts that loss of both Tbx5 and Tbx3 from the VCS will cause a transformation to a working myocardial phenotype. This can be evaluated in Tbx3 floxed mice (Frank, et al., 2012).

### Example 5

Briefly, transgenic mES cell lines were made with inducible overexpression of withe Tbx3 and Tbx5. It was found that they can be very efficiently differentiated into cardiomyocytes. The conduction system phenotypes caused by induced Tbx3 or Tbx5 expression have undergone initial investigation, and clear phenotypic changes have been observed consistent with altered conduction system phenotypes.

Dox inducible Tbx3 and Tbx5 ES cell lines. The Dox-inducible Tbx3 and Tbx5 ESC lines were made using the inducible cassette exchange method (Iacovino et al., Stem

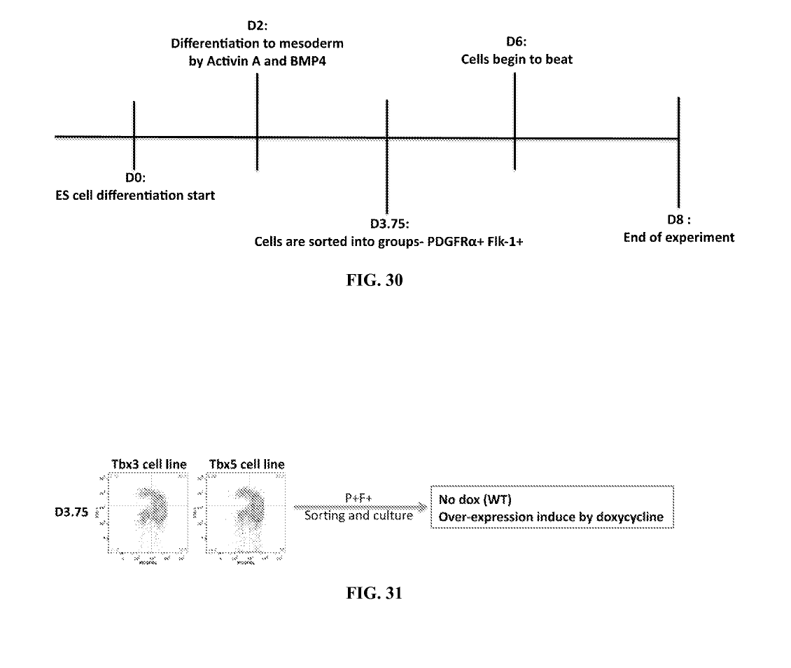

Cell, 2011). Briefly, a 3xFlag tag followed by two TEV protease recognizable sequences and an avidin tag was conjugated to the C-terminus of the mouse Tbx3 or Tbx5 gene. The tagged gene was subsequently cloned into the p2lox plasmid. The p2lox-Tbx3-3xFlag-2xTEV-Avi-IRES-BirA and p2lox-Tbx5-3xFlag-2xTEV-Avi-IRES-BirA plasmids were then transfected into the ZX1 mouse ESC by electroporation to generate the inducible Tbx3 and Tbx5 ESC lines. See FIGS. 30 and 31.

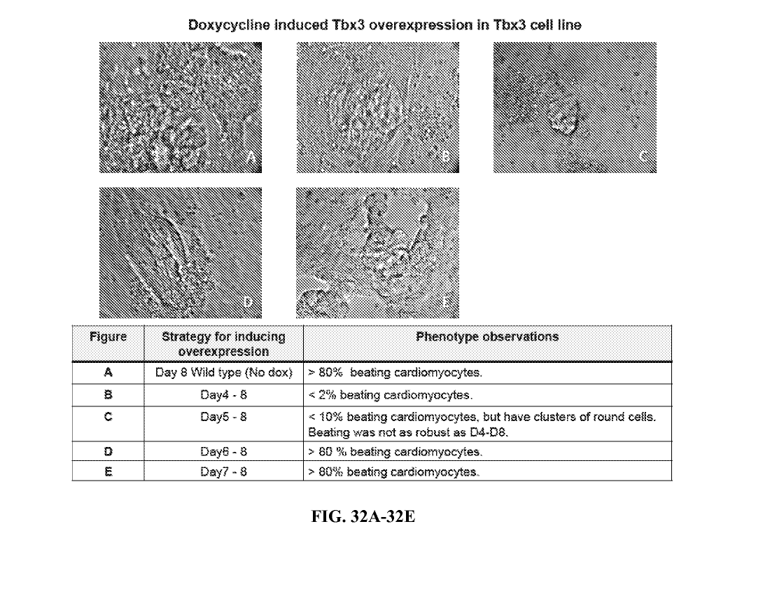

Both ES cell lines were successfully differentiated to cardiac precursor, marked by PDGFRa and Flk-1 expression, and the precursor cells PDGFRα+Flk-1+cells were sorted by flow cytometry, and then induced to cardiac lineage. See FIG. 32. Tbx3 ES cell line was successfully differentiated to beating cardiomyocytes (FIG. 32A, >80%). The Tbx3-overexpression induction during different periods by doxycycline showed phenotypical differences (FIGS. 32B-E).

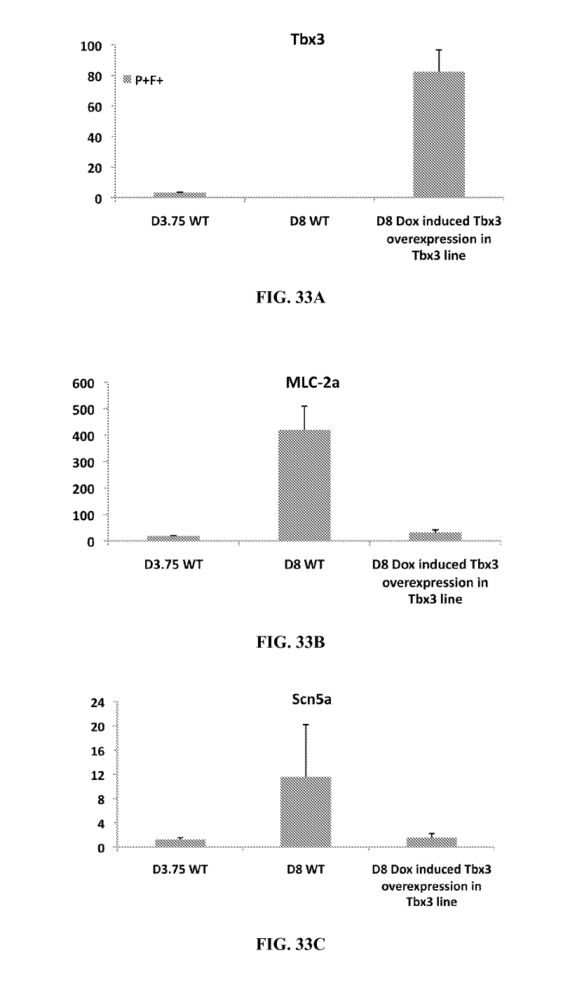

By Tbx3 over-expression induced by doxycycline, the expression level of Tbx3, slow conduction marker, was up-regulated. See FIG. 33A. By Tbx3 over-expression induced by doxycycline, the expression level of MLC-2a, myocardium marker, was up-regulated. See FIG. 33B. By Tbx3 over-expression induced by doxycycline, the expression level of SCN5a, conduction system marker, was down-regulated. See FIG. 33C.

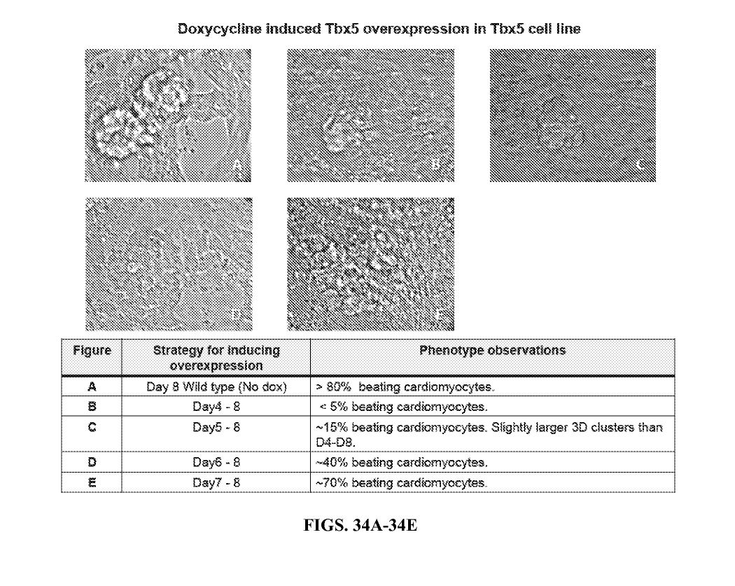

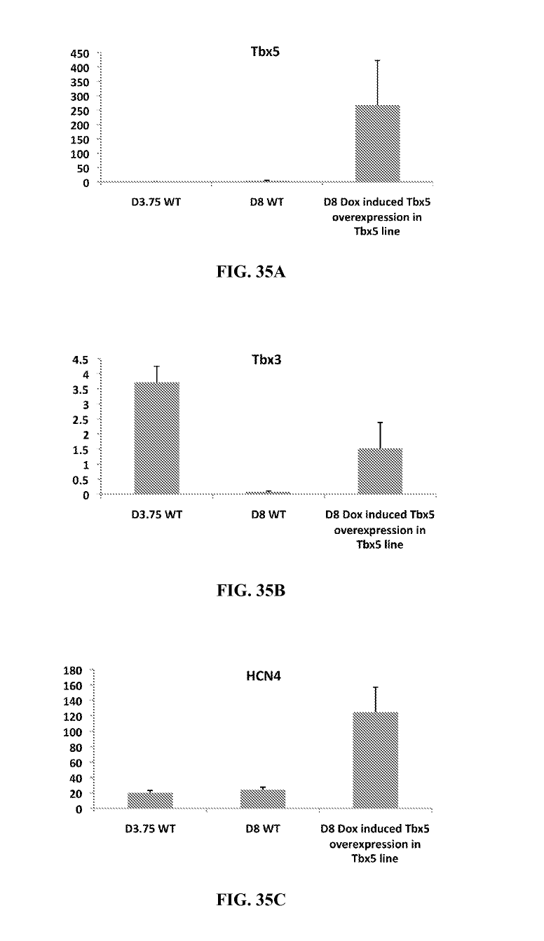

Tbx5 ES cell line was successfully differentiated to beating cardiomyocytes (FIG. 34A, >80%). The Tbx5-overexpression induction during different periods by doxycycline showed phenotypical differences (FIGS. 34B-E). By Tbx3 over-expression induced by doxycycline, the expression level of Tbx5, fast conduction system marker, was up-regulated. See FIG. 35A. By Tbx5 over-expression induced by doxycycline, the expression level of Tbx3 was up-regulated. See FIG. 35B. By Tbx5 over-expression induced by doxycycline, the expression level of HCN4, sa conduction system marker, was up-regulated. See FIG. 35C.

### References

The following references, to the extent that they provide exemplary procedural or other details supplementary to those set forth herein, are specifically incorporated herein by reference.
Aanhaanen W T, et al. Circ Res. 104(11):1267-1274, 2009.
Aanhaanen W T J, et al. Circulation Research. 107(6):728-736, 2010.
Abdelwahid E, Cell Tissue Res. 305(1):67-78, 2001.
Abrahamsen B, et al. Science. 321(5889):702-705, 2008.
Akopian A N, et al. Nat Neurosci. 2(6):541-548, 1999.
Akopian A N, et al., Nature. 379(6562):257-262, 1996.
Anderson R H, et al. Circulation Research.35(6):909-922, 1974.
Anderson R H, et al., Clin Anat. 22(1):99-113, 2009.
Aoki M, et al. Dev Biol. 301(1):218-226, 2007.
Arking D E, et al. PLoS One. 4(1):e4333, 2009.
Arnolds D E, et al., Birth Defects Res A Clin Mol Teratol. 91(6):578-585, 2011.
Arnolds D E, Genesis. 49(11):878-884, 2011.
Auden A, et al. Gene Expr Patterns. 6(8):964-970, 2006.
Azhar M, et al. Int J Biol Sci. 6(6):546-555, 2010.
Bakker M L, et al. Cardiovasc Res. 2012.
Bakker M L, et al. Circulation Research. 102(11):1340-1349, 2008.
Banerjee S K, et al., Cardiovasc Res. 84(1):111-118, 2009.
Baruscotti M, et al., Proc Natl Acad Sci USA. 108(4):1705-1710, 2011.
Basson C T, et al. N Engl J Med. 330(13):885-891, 1994.
Basson C T, et al. Nat Genet. 1997;15(1):30-35, 1997.
Bevilacqua L M, et al. J Intery Card Electrophysiol. 4(3):459-467, 2000.
Beyer S, Genesis. 49(2):83-91, 2010.
Bhuiyan Z A, et al. Circulation. 116(14):1569-1576, 2007.
Blasius A L, et al. Proc Natl Acad Sci USA. 108(48):19413-19418, 2012.
Blow M J, et al. Nat Genet. 42(9):806-810, 2010.
Bolstad B M, et al., Bioinformatics. 19(2):185-193, 2003.
Boogerd K J, et al. Cardiovasc Res. 78(3):485-493, 2008.
Boukens B J D, et al., Circulation Research. 104(1):19-31, 2009.
Briggs L E, et al. Res. 103(6):580-590, 2008.
Bruneau B G, et al. Cell. 106(6):709-721, 2001.
Cerrone M, et al., Heart Rhythm. 6(11):1652-1659, 2009.
Chambers J C, et al. Nat Genet. 42(2):149-152, 2010.
Chien K R, et al., Science. 322(5907):1494-1497, 2008.
Cho Y S, et al. Nat Genet. 41(5):527-534, 2009.
Christoffels V M, Dev Biol. 224(2):263-274, 2000.
Christoffels V M, et al., Circulation: Arrhythmia and Electrophysiology. 2(2):195-207, 2009.
Constam D B, J Cell Biol. 144(1):139-149, 1999.
Copeland N G, Jenkins N A, Nat Rev Genet. 2(10):769-779, 2001.
Costantini D L, et al. Cell. 123(2):347-358, 2005.
Davis B N, et al., Nature. 454(7200):56-61, 2008.
Davis D L, et al. Mech Dev. 108(1-2):105-119, 2001.
Davydov E V, et al. PLoS Comput Biol. 6(12):e1001025, 2010.
de Jonge H J M, et al. PLoS ONE. 2(9):e898, 2007.
Decher N, et al. Cell Physiol Biochem. 28(1):77-86, 2011.
Delot E C, Development. 130(1):209-220, 2003.
Derynck R, Zhang Y E. Nature.425(6958):577-584, 2003.
Dominguez J N, et al., Cardiovasc Res. 78(1):45-52, 2008.
Donner B C, et al. Basic Res Cardiol. 106(1):75-87, 2011.
Dunwoodie S L, Mech Dev. 72(1-2):27-40, 1998.
Efimov I R, et al. Anat Rec A Discov Mol Cell Evol Biol. 280(2):952-965, 2004.
El-Badawi A, et al., J Histochem Cytochem. 15(10):580-588, 1967.
Endo S, et al. Proc Natl Acad Sci USA. 106(9):3525-3530, 2009.
Euler-Taimor G, Cardiovasc Res. 69(1):15-25, 2006.
Facer P, et al. Int Heart J. 52(3):146-152, 2011.
Felipe A, Am J Physiol. 267(3 Pt 1):C700-705, 1994.
Filmus J, Genome Biol. 9(5):224, 2008.
Franco D, et al. Cardiovascular Research. 52(1):65-75, 2001.
Frank D U, et al. Proc Natl Acad Sci USA. 109(3):E154-163, 2012.
Gaborit N, et al. J Physiol. 582(Pt 2):675-693, 2007.
Gentleman R. Bioinformatics and computational biology using R and Bioconductor. 2005.
Ghosh T K, et al. Hum Mol Genet. 10(18):1983-1994, 2001.
Goetz S C, Development. 133(13):2575-2584, 2006.
Gorza L, Vitadello M. Circulation Research. 65(2):360-369, 1989.
Gourdie R G, Circ Res. 72(2):278-289, 1993.
Gourdie R G, et al. J Cell Sci. 105 (Pt 4)985-991, 1993.
Graham V, et al., Dev Dyn. 235(1):143-151, 2006.
Grant A O. Circ Arrhythm Electrophysiol. 2(2):185-194, 2009.
Greener I D, et al. Circ Arrhythm Electrophysiol. 2(3):305-315, 2009.
Greener I D, et al. J Mol Cell Cardiol. 50(4):642-651, 2011.
Greulich F, et al., Cardiovasc Res. 91(2):212-222, 2011.
Habets P E, et al. Genes Dev. 16(10):1234-1246, 2002.
Hagendorff A, Circulation. 99(11):1508-1515, 1999.
Hansmann G, et al. J Clin Invest. 118(5):1846-1857, 2008.
He A, et al., Proc Natl Acad Sci USA. 108(14):5632-5637, 2011.
He M-L, et al. Biochem Biophys Res Commun. 297(2):185-192, 2002.
Hellstrom M, Development. 126(14):3047-3055, 1999.
Herrmann S, EMBO J. 26(21):4423-4432, 2007.
Hoesl E, et al. J Mol Cell Cardiol. 45(1):62-69, 2008.
Hoffmann A D, et al., Development. 136(10):1761-1770, 2009.
Hollnagel A, J Biol Chem. 274(28):19838-19845, 1999.
Holm H, et al. Nat Genet. 42(2):117-122, 2010.
Honore E, et al. EMBO J. 10(10):2805-2811, 1991.
Hoogaars W M, Cell Mol Life Sci. 64(6):646-660, 2007.
Hoogaars W M, et al. Cardiovasc Res. 62(3):489-499, 2004.
Hoogaars W M, et al. Genes Dev. 21(9):1098-1112, 2007.
Horsthuis T, et al. Circ Res. 105(1):61-69, 2009.
Ikeda T, et al. Anat Embryol. 182(6):553-562, 1990.
Ivanov S V, et al. Am J Pathol. 165(3):1007-1018, 2004.
Jarvis M F, et al. Proc Natl Acad Sci USA. 104(20):8520-8525, 2007.
Jay P Y, et al. J Clin Invest. 113(8):1130-1137, 2004.
Jay P Y, J Cardiovasc Electrophysiol. 16(1):82-85, 2005.
Joseph E M. Dev Dyn. 231(4):720-726, 2004.
Kirchhoff S, et al. Curr Biol. 8(5):299-302, 1998.
Kléber A G, et al., Physiol Rev. 84(2):431-488, 2004.
Kondo R P, et al., Cardiovasc Electrophysiol. 14(4):383-391, 2003.
Kothary R, et al. Development. 105(4):707-714, 1989.
Kreuzberg M M, et al. Proc Natl Acad Sci USA. 103(15):5959-5964, 2006.
Kupershmidt S, et al. Circulation Research. 84(2):146-152, 1999.
Lamers W H, et al., Anat Rec. 217(4):361-370, 1987.
Leaf D E, et al. Circ Res. 103(9):1001-1008, 2008.
Lee E C, et al. Genomics. 2001;73(1):56-65, 2001.
Leoni A-L, et al. PLoS ONE. 5(2):e9298, 2010.
Li J, et al. Circ Res. 102(8):975-985, 2008.
Li Q Y, et al. Nat Genet. 15(1):21-29, 1997.
Liu F, et al. J Mol Cell Cardiol. 45(6):715-723, 2008.
Marionneau C, et al. J Physiol. 562(Pt 1):223-234, 2005.
McDermott D A, et al. Pediatr Res. 58(5):981-986, 2005.
Miquerol L, et al., Cardiovasc Res. 91(2):232-242, 2011.
Miyasaka Y, et al. Circulation. 114(2):119-125, 2006.
Monassier L, Pharmacol Ther. 128(3):559-567, 2010.
Moorman A F, et al., J Histochem Cytochem. 49(1):1-8, 2001.
Moorman A F, et al., Physiol Rev. 83(4):1223-1267, 2003.
Mori A D, et al. Dev Biol. 297(2):566-586, 2006.
Morley G E, et al. J Cardiovasc Electrophysiol. 10(10):1361-1375, 1999.
Moskowitz I, et al. Cell. 129(7):1365-1376, 2007.
Moskowitz I P, et al. Development. 131(16):4107-4116, 2004.
Moskowitz I P, et al. Proc Natl Acad Sci USA. 108(10):4006-4011, 2011.
Nagy A. Genesis. 26(2):99-109, 2000.
Narlikar L, et al. Genome Res. 20(3):381-392, 2010.
Newton-Cheh C, et al. BMC Med Genet. 8 Suppl 1S7, 2007.
Newton-Cheh C, et al. Nat Genet. 41(4):399-406, 2009.
Nogami A. Pacing Clin Electrophysiol. 34(5):624-650, 2011.
Ovcharenko I, et al., Nucleic Acids Res. 32(Web Server issue):W280-286, 2004.
Pallante B A, et al. Circ Arrhythm Electrophysiol. 3(2):186-194, 2010.
Papadatos G A, et al. Proc Natl Acad Sci USA. 99(9):6210-6215, 2002.
Pfeufer A, et al. Nat Genet. 41(4):407-414, 2009.
Pfeufer A, et al. Nat Genet. 42(2):153-159, 2010.
Plageman T F, Jr., et al., Dev Dyn. 235(10):2868-2880, 2006.
Plaster N M, et al. Cell. 105(4):511-519, 2001.
Priori, Circ Res. 107(4):451-456, 2010.
Qi X, et al. Developmental Biology. 311(1):136-146, 2007.
Remme C A, et al. Basic Res Cardiol. 104(5):511-522, 2009.
Rentschler S, et al. Development. 128(10):1785-1792, 2001.
Ritchie M E, et al. Bioinformatics. 23(20):2700-2707, 2007.
Rook M B, et al., Cardiovasc Res. 93(1):12-23, 2012.
Schott J J, et al. Science. 281(5373):108-111, 1998.
Schweickert A, et al. Dev Dyn. 237(12):3557-3564, 2008.
Shen T, et al. J Clin Invest. 121(12):4640-4654, 2011.
Simon A M, Curr Biol. 8(5):295-298, 1998.
Singh R, et al. Cell Mol Life Sci. 69(8):1377-1389, 2012.
Sirard C, et al. Genes & Development. 12(1):107-119, 1998.
Smith J G, et al. Heart Rhythm. 6(5):634-641, 2009.
Smith J G, et al. PLoS Genet. 7(2):e1001304, 2011.
Smyth G K. Stat Appl Genet Mol Biol. 3Article3, 2004.
Somi S, Anat Rec A Discov Mol Cell Evol Biol. 279(1):636-651, 2004.
Song L, et al. Circulation Research. 101(3):277-285, 2007.
Soriano P. Nat Genet. 21(1):70-71, 1999.
Sotoodehnia N, et al. Nat Genet. 42(12):1068-1076, 2010.
Srinivas S, et al. BMC Dev Biol. 14, 2001.
Stadtfeld M, Dev Biol. 302(1):195-207, 2007.
Stirling L C, et al. Pain. 113(1-2):27-36, 2005.
Stroud D M, et al. Circulation. 116(22):2535-2543, 2007.
Sugi Y, Developmental Biology. 269(2):505-518, 2004.
Suzuki M. Exp Physiol. 91(1):141-147, 2006.
Takeda M, et al. Lab Invest. 2009; 89(9):983-993, 2009.
Tamaddon H S, et al. Circ Res. 87(10):929-936, 2000.
Tao J, et al. Development. 132(5):1021-1034, 2005.
Valderrabano M, et al. Atrioventricular ring reentry in embryonic mouse hearts. Circulation. 2006; 114(6):543-549, 2006.
van der Bom T, et al. Nat Rev Cardiol. 8(1):50-60, 2011.
van Veen A A, et al., Cardiovascular Research. 51(2):217-229, 2001.
van Veen T A, et al. Circulation. 112(15):2235-2244, 2005.
van Wijk B, Cardiovasc Res. 74(2):244-255, 2007.
VanderBrink B A, et al. J Cardiovasc Electrophysiol. 11(11):1270-1276, 2000.
Verheule S, et al. J Cardiovasc Electrophysiol. 10(10):1380-1389, 1999.
Verzi M P, et al., Developmental Biology. 287(1):134-145, 2005.
Vijayaraman & Ellenbogen, Hurst's The Heart. 1020-54, 2008.
Virágh S, Challice C E. Developmental Biology. 56(2):382-396, 1977a.
Virágh S, Challice C E. Developmental Biology. 56(2):397-411, 1977b.
Viswanathan S, J Mol Cell Cardiol. 42(5):946-953, 2007.
Wakili R, et al., J Clin Invest. 121(8):2955-2968, 2011.
Wang J, Birth defects research Part A, Clinical and molecular teratology. 91(6):441-448, 2011.
Wenink A. J Anat. 121(Pt 3):617-631, 1976.
Wessels A, et al. Anat. Rec. 232(1):97-111, 1992.
Wheeler M T, et al. J Clin Invest. 113(5):668-675, 2004.
Wilde A A, Brugada R. Circ Res. 108(7):884-897, 2011.
Yamada M, Dev Biol. 228(1):95-105, 2000.
Yang L, et al. Development. 133(8):1575-1585, 2006.
Yang X, Proc Nat Acad Sci USA. 95(7):3667-3672, 1998.
Zhang L, et al. Circulation. 111(21):2720-2726, 2005.
Zhang S S, et al. Proc Natl Acad Sci USA. 108(33):13576-13581, 2011.
Zhou L, et al. J Cell Biochem. 90(2):339-346, 2003.
Zimmermann K, et al. Nature. 447(7146):855-858, 2007.

1. A method of making a fast conducting cardiomyocyte comprising:
(a) obtaining a cardiomyocte; and
(b) increasing Tbx5 in the cardiomyocyte thereby coverting the cardiomyocyte into a fast conducting cardiomyocyte.

2. The method of claim 1, wherein the cardiomyoctye is a primary cardiomyocyte or a cardiomyocyte cell line.

3. The method of any of claims 1-2, wherein obtaining the cardiomyocyte comprises differentiating an embryonic stem (ES) cell or an induced pluripotent stem (iPS) cell into a cardiomyocyte.

4. The method of any of claims 1-3, wherein increasing the Tbx5 comprises overexpressing Tbx5.

5. The method of claim 4, wherein overexpressing Tbx5 comprises upregulating the expression of an endogenous Tbx5 gene.

6. The method of claim 4, wherein increasing the Tbx5 comprises expressing an exogenous nucleic acid sequence encoding Tbx5 in the cardiomyocyte.

7. The method of claim 6, wherein the exogenous nucleic acid sequence encoding Tbx5 is a cDNA or an mRNA.

8. The method of claim 6, wherein the exogenous nucleic acid sequence encoding Tbx5 is introduced into the cell by a viral vector or electroporation.

9. The method of claim 8, wherein the viral vector is a retroviral vector.

10. The method of claim 7, wherein the exogenous nucleic acid sequence encoding Tbx5 is integrated into the genome of the cell.

11. The method of any of claims 1-3, wherein increasing Tbx5 comprises introducing Tbx5 polypeptide into the cell.

12. The method of claim 3, wherein the ES cells are human ES cells.

13. The method of claim 3, wherein the iPS cells are human iPS cells.

14. The method of any of claims 1-13, wherein the fast conducting cardiomyocytes are selected from the group consisting of atrioventricular bundle cells, bundle branch cells, and Purkinje cells.

15. The method of any of claims 1-14, further comprising separating the fast conducting cardiomyocytes from the cardiomyocytes.

16. A method of making a slow conducting nodal cell comprising:
(a) obtaining a cardiomyocte; and
(b) increasing Tbx3 in the cardiomyocyte thereby coverting the cardiomyocyte into a slow conducting nodal cell.

17. The method of claim 16, wherein the cardiomyoctye is a primary cardiomyocyte or a cardiomyocyte cell line.

18. The method of any of claims 16-17, wherein obtaining the cardiomyocyte comprises differentiating an embryonic stem (ES) cell or an induced pluripotent stem (iPS) cell into a cardiomyocyte.

19. The method of any of claims 16-18, wherein increasing the Tbx3 comprises overexpressing Tbx3.

20. The method of claim 19, wherein overexpressing Tbx3 comprises upregulating the expression of an endogenous Tbx3 gene.

21. The method of claim 19, wherein increasing the Tbx3 comprises expressing an exogenous nucleic acid sequence encoding Tbx3 in the cardiomyocyte.

22. The method of claim 21, wherein the exogenous nucleic acid sequence encoding Tbx3 is a cDNA or an mRNA.

23. The method of claim 21, wherein the exogenous nucleic acid sequence encoding Tbx3 is introduced into the cell by a viral vector or electroporation.

24. The method of claim 23, wherein the viral vector is a retroviral vector.

25. The method of claim 22, wherein the exogenous nucleic acid sequence encoding Tbx3 is integrated into the genome of the cell.

26. The method of any of claims 16-18, wherein increasing Tbx3 comprises introducing Tbx3 polypeptide into the cell.

27. The method of claim 18, wherein the ES cells are human ES cells.

28. The method of claim 18, wherein the iPS cells are human iPS cells.

29. The method of any of claims 16-28, wherein the slow nodal cells are selected from the group consisting of AV nodal cells and SA nodal cells.

30. The method of any of claims 16-29, further comprising separating the slow nodal cells from the cardiomyocytes.

31. A method of screening a compound for an effect on a fast conducting cardiomyocyte cell culture, comprising:
(a) contacting the cell culture with the compound;
(b) observing a change in a fast conducting cardiomyocyte in the cell culture compared to a fast conducting cardiomyocyte in an untreated cell culture and
(c) determining a difference between the treated and untreated fast conducting cardiomyocytes in the respective cell cultures.

32. The method of claim 31, wherein the fast conducting cardiomyocyte is selected from the group consisting of atrioventricular bundle cells, bundle branch cells, and Purkinje cells.

33. The method of any of claims 31-32, wherein the fast conducting cardiomyocytes overexpress Tbx5.

34. The method of any of claims 31-33, wherein the compound is a drug.

35. The method of claim 31, wherein the drug is calcium channel blocker, a β-adrenoreceptor agonist, or an α-adrenoreceptor agonist.

36. The method of any of claims 31-33, wherein the compound is a peptide.

37. The method of any of claims 31-33, wherein the compound is an oligonucleotide.

38. The method of any of claims 31-33, wherein the compound is a toxin.

39. A method of screening a compound for an effect on a slow conducting nodal cell culture, comprising:
(a) contacting the cell culture with the compound;
(b) observing a change in a nodal cell in the cell culture compared to a nodal cell in an untreated cell culture; and
(c) determining a difference between the treated and untreated nodal cells in the respective cell cultures.

40. The method of claim 39, wherein the nodal cell is selected from the group consisting of AV nodal cells and SA nodal cells.

41. The method of claim 39, wherein the nodal cell overexpresses Tbx3.

42. The method of any of claims 39-41, wherein the compound is a drug.

43. The method of claim 42, wherein the drug is calcium channel blocker, a β-adrenoreceptor agonist, or an α-adrenoreceptor agonist.

44. The method of any of claims 39-41, wherein the compound is a peptide.

45. The method of any of claims 39-41, wherein the compound is an oligonucleotide.

46. The method of any of claims 39-41, wherein the compound is a toxin.

47. A method for treating a subject with heart disease, comprising transplanting the fast conducting cardiomyocyte produced according to any of claims 1-15 to the heart of the subject.

48. The method of claim 47, wherein the subject is a human.

49. The method of any of claims 47-48, wherein the cells were differentiated from iPS cells.

50. The method of claim 49, wherein the iPS cells were derived from the subject's somatic cells.

51. A method for treating a subject with heart disease, comprising transplanting the nodal cells produced according to claim 16 to the heart of the subject.

52. The method of claim 51, wherein the subject is a human.

53. The method of any of claims 51-52, wherein the cells were differentiated from iPS cells.

54. The method of claim 53, wherein the iPS cells were derived from the subject's somatic cells.

55. A fast conducting cardiomyocyte comprising an exogenous nucleic acid sequence encoding Tbx5, wherein expression of Tbx5 is increased as compared to a cardiomyocyte that does not have the exogeneous nucleic acid sequence.

56. The fast conducting cardiomyocyte of claim 55, wherein the cardiomyocyte is derived from an embryonic stem (ES) cell or an induced pluripotent stem (iPS) cell.

57. The fast conducting cardiomyocyte of claim 55, wherein the cardiomyoctye is a primary cardiomyocyte or a cardiomyocyte cell line.

58. The fast conducting cardiomyocyte of any of claims 55-57, wherein the exogenous nucleic acid sequence encoding Tbx5 is a cDNA or an mRNA.

59. The fast conducting cardiomyocyte of of any of claims 55-58, wherein the exogenous nucleic acid sequence encoding Tbx5 is integrated into the genome of the cell.

61. The fast conducting cardiomyocyte of claim 56, wherein the ES cells are human ES cells.

62. The fast conducting cardiomyocyte of claim 56, wherein the iPS cells are human iPS cells.

63. The fast conducting cardiomyocyte of any of claims 55-62, wherein the fast conducting cardiomyocyte is selected from the group consisting of atrioventricular bundle cells, bundle branch cells, and Purkinje cells.

64. A slow conducting nodal cell comprising an exogenous nucleic acid sequence encoding Tbx3, wherein expression of Tbx3 is increased as compared to a cardiomyocyte that does not have the exogeneous nucleic acid sequence.

65. The slow conducting nodal cell of claim 64, wherein the cardiomyocyte is derived from an embryonic stem (ES) cell or an induced pluripotent stem (iPS) cell.

66. The slow conducting nodal cell of claim 64, wherein the cardiomyoctye is a primary cardiomyocyte or a cardiomyocyte cell line.

67. The slow conducting nodal cell of any of claims 64-66, wherein the exogenous nucleic acid sequence encoding Tbx3 is a cDNA or an mRNA.

68. The slow conducting nodal cell of of any of claims 64-67, wherein the exogenous nucleic acid sequence encoding Tbx3 is integrated into the genome of the cell.

69. The slow conducting nodal cell of claim 65, wherein the ES cells are human ES cells.

70. The slow conducting nodal cell of claim 65, wherein the iPS cells are human iPS cells.

71. The slow conducting nodal cell of any of claims 64-70, wherein the fast conducting cardiomyocyte is selected from the group consisting of AV nodal cells and SA nodal cells.
